(19) United States
(12) Patent Application Publication
(10) Pub. No.: US 2011/0151526 A1
(43) Pub. Date: Jun. 23, 2011

(54) Branched-Chain Fatty Acids And Biological Production Thereof

(76) Inventors: Charles Winston Saunders, Fairfield, OH (US); Jun Xu, Mason, OH (US); Phillip Richard Green, Wyoming, OH (US); David Blair Cody, West Harrison, OH (US); Zubin Saresh Khambatta, Fairfield, OH (US)

(21) Appl. No.: 12/972,822

(22) Filed: Dec. 20, 2010

Related U.S. Application Data
(60) Provisional application No. 61/289,039, filed on Dec. 22, 2009.

(51) Int. Cl. C12P 7/64; C12N 1/21

(52) U.S. Cl. 435/134; 435/252.33

(57) ABSTRACT

A method for producing anteiso fatty acid is provided. The method comprises culturing a cell comprising at least one exogenous or overexpressed polynucleotide comprising a nucleic acid sequence encoding a polypeptide that catalyzes at least one of the following reactions: conversion of pyruvate to citramalate; conversion of citramalate to citraconate; conversion of citraconate to β-methyl-D-malate; conversion of β-methyl-D-malate to 2-oxobutanoate; or conversion of threonine to 2-oxobutanoate, under conditions allowing expression of the polynucleotide(s) and production of anteiso fatty acid. Optionally the cell further comprises at least one exogenous or overexpressed polynucleotide comprising a nucleic acid sequence encoding a polypeptide that catalyzes conversion of 2-oxobutanoate to 2-aceto-2-hydroxy-butyrate, conversion of 2-aceto-2-hydroxy-butyrate to 2,3-dihydroxy-3-methylvalerate, and/or conversion of 2,3-dihydroxy-3-methylvalerate to α-keto-3-methylvalerate. A cell that produces anteiso fatty acid and a method of using the cell to produce anteiso fatty acid also are provided.

### CROSS REFERENCE TO RELATED APPLICATIONS

This application claims priority to U.S. Provisional Application No. 61/289,039, filed Dec. 22, 2009, which is hereby incorporated by reference in its entirety.

### FIELD OF THE INVENTION

The invention relates to cells and methods for producing fatty acids, and more particularly relates to cells and methods for producing anteiso and/or iso branched-chain fatty acids.

### BACKGROUND OF THE INVENTION

Anteiso and iso branched-chain fatty acids are carboxylic acids with a methyl branch on the n-2 and n-1 carbon, respectively. Similar to other fatty acids, anteiso and iso branched-chain fatty acids are useful in manufacturing, such as, e.g., food, detergents, pesticides, and personal care products such as shampoos, soaps, and cosmetics.

Anteiso and iso branched-chain fatty acids can be chemically synthesized or can be isolated from certain animals and bacteria. While certain bacteria, such as Escherichia coli, do not naturally produce anteiso or iso branched-chain fatty acids, some bacteria, such as members of the genera Bacillus and Streptomyces, do naturally produce anteiso and iso branched-chain fatty acids. For example, Streptomyces avermitilis and Bacillus subtilis both produce anteiso fatty acids with 15 and 17 total carbons and iso branched fatty acids with 15, 16 and 17 total carbons (Cropp et al., Can. J. Microbiology 46: 506-14 (2000); De Mendoza et al., Biosynthesis and Function of Membrane Lipids, in Bacillus subtilis and Other Gram-Positive Bacteria, Sonenshein and Losick, eds., American Society for Microbiology, (1993)). However, these organisms do not produce anteiso and/or iso branched-chain fatty acids in amounts that are commercially useful. Another limitation of these natural organisms is that they apparently do not produce medium-chain anteiso and/or iso branched-chain fatty acids, such as those with 11 or 13 carbons.

As such, there remains a need for commercially useful biosynthetically-produced anteiso and/or iso branched-chain fatty acids. In addition, there remains a need for a method of producing such anteiso and/or iso branched-chain fatty acids.

### SUMMARY OF THE INVENTION

Cells and methods for producing anteiso and/or iso branched-chain fatty acids (also referred to herein as anteiso and/or iso fatty acids) are provided. The polynucleotide comprises a nucleic acid sequence encoding a polypeptide that catalyzes a reaction associated with branched-chain fatty acid production in the cell.

In one aspect, the invention provides a method for producing anteiso fatty acid. The method comprises culturing a cell comprising at least one exogenous or overexpressed polynucleotide comprising a nucleic acid sequence encoding a polypeptide that catalyzes at least one of the following reactions: (aa) conversion of pyruvate to citramalate; (bb) conversion of citramalate to citraconate; (cc) conversion of citraconate to β-methyl-D-malate; (dd) conversion of β-methyl-D-malate to 2-oxobutanoate; or (ee) conversion of threonine to 2-oxobutanoate, under conditions allowing expression of the polynucleotide(s) and production of anteiso fatty acid. The cell produces more anteiso fatty acids than an otherwise similar cell that does not comprise the polynucleotide(s). In some embodiments, the cell further comprises at least one exogenous or overexpressed polynucleotide comprising a nucleic acid sequence encoding a polypeptide that catalyzes at least one of the following reactions: (ff) conversion of 2-oxobutanoate to 2-aceto-2-hydroxy-butyrate, (gg) conversion of 2-aceto-2-hydroxy-butyrate to 2,3-dihydroxy-3-methylvalerate, or (hh) conversion of 2,3-dihydroxy-3-methylvalerate to 2-keto-3-methylvalerate. Optionally, the method further comprises extracting anteiso fatty acid from the culture or extracting from the culture a product derived from anteiso fatty acid.

The invention also provides a cell comprising an exogenous or overexpressed polynucleotide comprising a nucleic acid sequence encoding a threonine deaminase, an exogenous or overexpressed polynucleotide comprising a nucleic acid sequence encoding a branched-chain α-keto acid dehydrogenase, and an exogenous or overexpressed polynucleotide comprising a nucleic acid sequence encoding a 3-ketoacyl-ACP synthase, wherein the polynucleotides are expressed in the cell. Additionally, the invention provides a cell comprising an exogenous or overexpressed polynucleotide comprising a nucleic acid sequence encoding a citramalate synthase, an exogenous or overexpressed polynucleotide comprising a nucleic acid sequence encoding a branched-chain α-keto acid dehydrogenase, and an exogenous or overexpressed polynucleotide comprising a nucleic acid sequence encoding a 3-ketoacyl-ACP synthase, wherein the polynucleotides are expressed in the cell. Optionally, the cell further comprises an exogenous or overexpressed polynucleotide comprising a nucleic acid sequence encoding an acetohydroxy acid synthase and/or an exogenous or overexpressed polynucleotide comprising a nucleic acid sequence encoding a thioesterase. A method of producing anteiso fatty acid by culturing the cell also is provided.

In certain embodiments, a cell having at least one exogenous polynucleotide is provided. Alternatively or in addition, the cell comprises a polynucleotide that is overexpressed. The polynucleotide has a nucleic acid sequence encoding a polypeptide that catalyzes one of the following reactions: conversion of isoleucine to 2-keto, 3-methylvalerate; conversion of 2-keto, 3-methylvalerate to 2-methylbutyryl-CoA; conversion of 2-methylbutyryl-CoA to 2-methylbutyryl-ACP; conversion of 2-methylbutyryl-ACP to 4-methyl 3-ketohexanoyl-ACP; conversion of 2-methylbutyryl-CoA to 4-methyl 3-ketohexanoyl-ACP; or conversion of acyl-ACP to anteiso fatty acids. The cell comprising the exogenous polynucleotide produces more anteiso fatty acids than an otherwise similar cell that does not comprise the exogenous polynucleotide.

A method of increasing anteiso fatty acids in a bacterial cell is also provided. The method includes expressing in a bacterial cell a polynucleotide encoding a polypeptide that catalyzes one of the following reactions: conversion of 2-keto, 3-methylvalerate to 2-methylbutyryl-CoA; conversion of 2-methylbutyryl-CoA to 2-methylbutyryl-ACP; conversion of 2-methylbutyryl-ACP to 4-methyl 3-ketohexanoyl-ACP; conversion of 2-methylbutyryl-CoA to 4-methyl 3-ketohexanoyl-ACP; or conversion of acyl-ACP to anteiso fatty acids, and culturing the bacterial cell under conditions that allow the cell to produce the polypeptide such that anteiso fatty acids are produced.

Further provided is an Escherichia coli cell that produces anteiso fatty acids.

Also provided is a method of increasing anteiso fatty acids in a cell. The method includes expressing in a cell a polynucleotide encoding an exogenous branched-chain amino acid aminotransferase, an exogenous branched-chain α-keto acid dehydrogenase (BCDH), and an exogenous 3-ketoacyl-ACP synthase; and culturing the cell under conditions such that anteiso fatty acids are produced.

In certain embodiments, a method for making anteiso fatty acids is provided. The method includes culturing at least one cell comprising at least one exogenous polynucleotide that encodes at least one polypeptide that is capable of producing anteiso fatty acids from isoleucine under conditions such that anteiso fatty acids are produced.

In addition, in certain embodiments, a cell comprising at least two exogenous polynucleotides is also provided. The exogenous polynucleotides comprise nucleic acid sequences encoding polypeptides that catalyze at least two of the following reactions: conversion of leucine to 2-keto, 4-methylvalerate; conversion of valine to 2-keto 3-methylbutyrate; conversion of 2-keto, 4-methylvalerate to 3-methylbutyryl-CoA; conversion of 3-methylbutyryl-CoA to 3-methylbutyryl-ACP; conversion of 3-methylbutyryl-ACP to 5-methyl 3-ketohexanoyl-ACP; conversion of 2-keto 3-methylbutyrate to 2-methylpropionyl-CoA; conversion of 2-methylpropionyl-CoA to 2-methylpropionyl-ACP; conversion of 2-methylpropionyl-ACP to 4-methylvaleroyl-ACP; conversion of 3-methylbutyryl-CoA to 5-methyl 3-ketohexanoyl-ACP; conversion of 2-methylpropionyl-CoA to 4-methyl 3-ketovaleroyl-ACP; or conversion of acyl-ACP to iso fatty acids, and wherein the cell comprising the exogenous polynucleotides produces more iso fatty acids than an otherwise similar cell that does not comprise the exogenous polynucleotides.

Also provided is a method for increasing iso fatty acids in a bacterial cell. The method includes expressing in a bacterial cell polynucleotides encoding at least two polypeptides, the polypeptides catalyzing at least two of the following reactions: conversion of leucine to 2-keto, 4-methylvalerate; conversion of valine to 2-keto 3-methylbutyrate; conversion of 2-keto, 4-methylvalerate to 3-methylbutyryl-CoA; conversion of 3-methylbutyryl-CoA to 3-methylbutyryl-ACP; conversion of 3-methylbutyryl-ACP to 5-methyl 3-ketohexanoyl-ACP; conversion of 2-keto 3-methylbutyrate to 2-methylpropionyl-CoA; conversion of 2-methylpropionyl-CoA to 2-methylpropionyl-ACP; conversion of 2-methylpropionyl-ACP to 4-methylvaleroyl-ACP; conversion of 3-methylbutyryl-CoA to 5-methyl 3-ketohexanoyl-ACP; conversion of 2-methylpropionyl-CoA to 4-methyl 3-ketovaleroyl-ACP; or conversion of acyl-ACP to iso fatty acids, and culturing the bacterial cell under conditions that allow the cell to produce the polypeptides, such that iso fatty acids are produced.

The following numbered paragraphs each succinctly define one or more exemplary variations of the invention:

1. A method for producing anteiso fatty acid, the method comprising culturing a cell comprising at least one exogenous or overexpressed polynucleotide comprising a nucleic acid sequence encoding a polypeptide that catalyzes at least one of the following reactions: (aa) conversion of pyruvate to citramalate; (bb) conversion of citramalate to citraconate; (cc) conversion of citraconate to β-methyl-D-malate; (dd) conversion of β-methyl-D-malate to 2-oxobutanoate; or (ee) conversion of threonine to 2-oxobutanoate under conditions allowing expression of the polynucleotide(s) and production of anteiso fatty acid, wherein the cell produces more anteiso fatty acids than an otherwise similar cell that does not comprise the polynucleotide(s).

2. The method of paragraph 1, further comprising exposing the cell to thiamine

3. The method of paragraph 1, further comprising extracting anteiso fatty acid from the culture.

4. The method of paragraph 1, further comprising extracting from the culture a product derived from anteiso fatty acid.

5. The method of paragraph 1, wherein the cell further comprises at least one exogenous or overexpressed polynucleotide comprising a nucleic acid sequence encoding a polypeptide that catalyzes at least one of the following reactions: (ff) conversion of 2-oxobutanoate to 2-aceto-2-hydroxy-butyrate, (gg) conversion of 2-aceto-2-hydroxy-butyrate to 2,3-dihydroxy-3-methylvalerate, or (hh) conversion of 2,3-dihydroxy-3-methylvalerate to 2-keto-3-methylvalerate.

6. The method of paragraph 5, wherein the cell comprises exogenous or overexpressed polynucleotides encoding polypeptides that catalyze 3, 4, 5, 6, 7, or all of the reactions.

7. The method of paragraph 5, wherein the cell comprises exogenous or overexpressed polynucleotides encoding polypeptides that catalyze reactions (aa), (bb), (cc), and (ff).

8. The method of paragraph 5, wherein the cell comprises an exogenous or overexpressed polynucleotide encoding a citramalate synthase, an exogenous or overexpressed polynucleotide comprising a nucleic acid sequence encoding an acetohydroxy acid synthase, an exogenous or overexpressed polynucleotide comprising a nucleic acid sequence encoding an isopropylmalate isomerase, an exogenous or overexpressed polynucleotide comprising a nucleic acid sequence encoding an isopropylmalate dehydrogenase, or a combination thereof.

9. The method of paragraph 8, wherein the cell comprises an exogenous polynucleotide encoding a citramalate synthase, an exogenous or overexpressed polynucleotide encoding an acetohydroxy acid synthase, an exogenous or overexpressed polynucleotide encoding an isopropylmalate isomerase, and an exogenous or overexpressed polynucleotide encoding an isopropylmalate dehydrogenase.

10. The method of paragraph 8, wherein the citramalate synthase is CimA derived from M. jannaschii.

11. The method of paragraph 8, wherein the isopropylmalate isomerase is E. coli LeuCD.

12. The method of paragraph 8, wherein the isopropylmalate dehydrogenase is E. coli LeuB.

13. The method of paragraph 8, wherein the acetohydroxy acid synthase is E. coli IlvIH, E. coli IlvIH (G14D), E. coli IlvGM, or B. subtilis IlvBH.

14. The method of paragraph 5, wherein the cell comprises exogenous or overexpressed polynucleotides encoding polypeptides that catalyze reactions (ee) and (ff).

15. The method of paragraph 5, wherein the cell comprises an exogenous or overexpressed polynucleotide encoding a threonine deaminase and an exogenous or overexpressed polynucleotide encoding an acetohydroxy acid synthase.

16. The method of paragraph 15, wherein the threonine deaminase is E. coli TdcB.

17. The method of paragraph 15, wherein the acetohydroxy acid synthase is E. coli IlvIH, E. coli IlvIH (G14D), E. coli IlvGM, or B. subtilis IlvBH.

18. The method of paragraph 5, wherein the one or more of the exogenous or overexpressed polynucleotides (i) comprise a nucleic acid sequence having at least about 90 percent identity to the nucleic acid sequence set forth in SEQ ID NO: 32, 36, 42, 43, 46, 51, 57, 62, 68, or 83, or (ii) encode a polypeptide comprising an amino acid sequence having at least about 90 percent identity to the amino acid sequence set forth in SEQ ID NO: 33, 39, 40, 41, 47, 48, 52, 53, 58, 65, 66, 67, 84, or 85.

19. The method of paragraph 5, wherein the cell is modified to attenuate branched-chain amino acid aminotransferase activity.

20. The method of paragraph 5, wherein the cell further comprises an exogenous or overexpressed polynucleotide comprising a nucleic acid sequence encoding a branched-chain amino acid aminotransferase, an exogenous or overexpressed polynucleotide comprising a nucleic acid sequence encoding a branched-chain α-keto acid dehydrogenase, an exogenous or overexpressed polynucleotide comprising a nucleic acid sequence encoding an acyl transferase, an exogenous or overexpressed polynucleotide comprising a nucleic acid sequence encoding a 3-ketoacyl-ACP synthase, an exogenous or overexpressed polynucleotide comprising a nucleic acid sequence encoding an enoyl-ACP reductase, an exogenous or overexpressed polynucleotide comprising a nucleic acid sequence encoding a thioesterase, or a combination thereof.

21. The method of paragraph 20, wherein one or more of the exogenous or overexpressed polynucleotides comprise a nucleic acid sequence (i) having at least 90 percent identity to the nucleic acid sequence set forth in SEQ ID NO: 1, 4, 7, 13, 17, 18, 19, 20, 21, 22, 23, 68, 77, or 78 or (ii) encoding a polypeptide having an amino acid sequence having at least 90 percent identity to the amino acid sequence set forth in SEQ ID NO: 10, 16, 24, 25, 26, 27, 28, 29, or 73.

22. The method of paragraph 20, wherein the cell comprises an exogenous or overexpressed polynucleotide comprising a nucleic acid sequence encoding a branched-chain α-keto acid dehydrogenase and an exogenous or overexpressed polynucleotide comprising a nucleic acid sequence encoding a 3-ketoacyl-ACP synthase.

23. The method of paragraph 22, wherein the cell is an Escherichia cell.

24. The method of paragraph 22, wherein the branched-chain α-keto acid dehydrogenase is B. subtilis Bkd.

25. The method of paragraph 22, wherein the 3-ketoacyl-ACP synthase is B. subtilis FabH.

26. The method of paragraph 22, wherein the cell further comprises an exogenous or overexpressed polynucleotide comprising a nucleic acid sequence encoding a thioesterase.

27. A cell comprising: an exogenous or overexpressed polynucleotide comprising a nucleic acid sequence encoding a threonine deaminase, an exogenous or overexpressed polynucleotide comprising a nucleic acid sequence encoding a branched-chain α-keto acid dehydrogenase, and an exogenous or overexpressed polynucleotide comprising a nucleic acid sequence encoding a 3-ketoacyl-ACP synthase, wherein the polynucleotides are expressed and the cell produces more anteiso fatty acid than an otherwise similar cell that does not comprise the polynucleotide(s).

28. The cell of paragraph 27, wherein the branched-chain α-keto acid dehydrogenase is B. subtilis Bkd and the 3-ketoacyl-ACP synthase is B. subtilis FabH.

29. The cell of paragraph 27 further comprising an exogenous or overexpressed polynucleotide comprising a nucleic acid sequence encoding a thioesterase.

30. The cell of paragraph 27 further comprising an exogenous or overexpressed polynucleotide comprising a nucleic acid sequence encoding an acetohydroxy acid synthase.

31. The cell of paragraph 30, wherein the acetohydroxy acid synthase is E. coli IlvIH, E. coli IlvIH (G14D), E. coli IlvGM, or B. subtilis IlvBH.

32. The cell of paragraph 27, wherein the cell is a bacterial cell that does not naturally produce anteiso fatty acids

33. The cell of paragraph 32, wherein the cell is an Escherichia cell.

34. A method of producing anteiso fatty acid, the method comprising culturing the bacterial cell of paragraph 32 under conditions that allow expression of the polynucleotides and production of anteiso fatty acid.

35. A cell comprising: an exogenous or overexpressed polynucleotide comprising a nucleic acid sequence encoding a citramalate synthase, an exogenous or overexpressed polynucleotide comprising a nucleic acid sequence encoding a branched-chain α-keto acid dehydrogenase, and an exogenous or overexpressed polynucleotide comprising a nucleic acid sequence encoding a 3-ketoacyl-ACP synthase, wherein the polynucleotides are expressed and the cell produces more anteiso fatty acid than an otherwise similar cell that does not comprise the polynucleotide(s).

36. The cell of paragraph 35, wherein the citramalate synthase is CimA derived from M. jannaschii, the branched-chain α-keto acid dehydrogenase is B. subtilis Bkd, and the 3-ketoacyl-ACP synthase is B. subtilis FabH.

37. The cell of paragraph 35 further comprising an exogenous or overexpressed polynucleotide comprising a nucleic acid sequence encoding an isopropylmalate isomerase, an exogenous or overexpressed polynucleotide comprising a nucleic acid sequence encoding an isopropylmalate dehydrogenase, an exogenous or overexpressed polynucleotide comprising a nucleic acid sequence encoding an acetohydroxy acid synthase, an exogenous or overexpressed polynucleotide comprising a nucleic acid sequence encoding an enoyl-ACP synthase, an exogenous or overexpressed polynucleotide comprising a nucleic acid sequence encoding a thioesterase, or a combination thereof.

38. The method of paragraph 37, wherein the isopropylmalate isomerase is E. coli LeuCD.

39. The method of paragraph 37, wherein the isopropylmalate dehydrogenase is E. coli LeuB.

40. The cell of paragraph 37, wherein the acetohydroxy acid synthase is E. coli IlvIH, E. coli IlvIH (G14D), E. coli IlvGM, or B. subtilis IlvBH.

41. The cell of paragraph 35, wherein the cell is a bacterial cell that does not naturally produce anteiso fatty acids.

42. The cell of paragraph 41, wherein the cell is an Escherichia cell.

43. A method of producing anteiso fatty acid, the method comprising culturing a bacterial cell of paragraph 41 under conditions that allow expression of the polynucleotides and production of anteiso fatty acid.

44. A cell comprising at least one exogenous polynucleotide, wherein the polynucleotide comprises a nucleic acid sequence encoding a polypeptide that catalyzes one of the following reactions: a. conversion of isoleucine to 2-keto, 3-methylvalerate; b. conversion of 2-keto, 3-methylvalerate to 2-methylbutyryl-CoA; c. conversion of 2-methylbutyryl-CoA to 2-methylbutyryl-ACP; d. conversion of 2-methylbutyryl-ACP to 4-methyl 3-ketohexanoyl-ACP; e. conversion of 2-methylbutyryl-CoA to 4-methyl 3-ketohexanoyl-ACP; or f. conversion of acyl-ACP to anteiso fatty acids, and wherein the cell comprising the exogenous polynucleotide produces more anteiso fatty acids than an otherwise similar cell that does not comprise the exogenous polynucleotide.

45. The cell of paragraph 44, wherein the polynucleotide encodes a branched-chain amino acid aminotransferase, a branched-chain α-keto acid dehydrogenase (BCDH), an acyl transferase, a 3-ketoacyl-ACP synthase, or a thioesterase.

46. The cell of paragraph 44, wherein the cell is an Escherichia cell.

47. The cell of paragraph 44, wherein the cell comprising polynucleotides encoding polypeptides that catalyze 2, 3, 4, 5, or all of the reactions.

48. The cell of paragraph 44, wherein the polynucleotide has at least 30 percent sequence identity to a sequence set forth in SEQ ID NO: 1, 4, 7, 13, 17, 18, 19, 20, 21, 22, or 23.

49. The cell of paragraph 44, wherein the polypeptide has at least 40 percent sequence identity to a sequence set forth in SEQ ID NO: 10, 16, 24, 25, 26, 27, 28, or 29.

50. The cell of paragraph 44, wherein the cell is an Escherichia coli cell and the polynucleotide has at least 65 percent sequence identity to a sequence set forth in SEQ ID NO: 1, 4, 7, 13, 17, 18, 19, 20, 21, 22, or 23.

51. The cell of paragraph 44, wherein the polypeptide is substantially identical to a polypeptide having the sequence set forth in SEQ ID NO: 10, 16, 24, 25, 26, 27, 28, or 29.

52. The cell of paragraph 44, wherein the anteiso fatty acids are medium-chain anteiso fatty acids.

53. The cell of paragraph 44, wherein the anteiso fatty acids are not naturally produced in the cell.

54. Anteiso fatty acids produced by the cell of paragraph 44.

55. The cell of paragraph 44, wherein the polynucleotide encodes a fatty acid synthase gene from a Bacillus, a Streptomyces, or a Listeria.

56. The cell of paragraph 44, wherein the polynucleotide encodes a fatty acid synthase gene from an organism that naturally produces branched-chain fatty acids.

57. A method of increasing anteiso fatty acids in a bacterial cell, comprising:

a. expressing in a bacterial cell a polynucleotide encoding a polypeptide that catalyzes one of the following reactions: i. conversion of 2-keto, 3-methylvalerate to 2-methylbutyryl-CoA; ii. conversion of 2-methylbutyryl-CoA to 2-methylbutyryl-ACP; iii. conversion of 2-methylbutyryl-ACP to 4-methyl 3-ketohexanoyl-ACP; iv. conversion of 2-methylbutyryl-CoA to 4-methyl 3-ketohexanoyl-ACP; or v. conversion of acyl-ACP to anteiso fatty acids, and

b. culturing the bacterial cell under conditions that allow the cell to produce the polypeptide, such that anteiso fatty acids are produced.

58. The method of paragraph 57, wherein the cell produces higher levels of anteiso fatty acids after expression of the polynucleotide than it did prior to expression of the polynucleotide.

59. The method of paragraph 57, wherein the polypeptide is a branched-chain amino acid aminotransferase, a branched-chain α-keto acid dehydrogenase (BCDH), a 3-ketoacyl-ACP synthase, or a thioesterase.

60. The method of paragraph 57, wherein the cell is an Escherichia cell.

61. The method of paragraph 57, wherein the method includes expressing in the bacterial cell polynucleotides encoding polypeptides that catalyze 2, 3, 4, 5, or all of the reactions.

62. The method of paragraph 57, wherein the polynucleotide has at least 30 percent sequence identity to a sequence set forth in SEQ ID NO: 1, 4, 7, 13, 17, 18, 19, 20, 21, 22, or 23.

63. The method of paragraph 57, wherein the polypeptide has at least 40 percent sequence identity to a sequence set forth in SEQ ID NO: 10, 16, 24, 25, 26, 27, 28, or 29.

64. The method of paragraph 57, wherein the cell is an Escherichia coli cell and the polynucleotide has at least 65 percent sequence identity to a sequence set forth in SEQ ID NO: 1, 4, 7, 13, 17, 18, 19, 20, 21, 22, or 23.

65. The method of paragraph 57, wherein the polypeptide is substantially identical to a polypeptide having the sequence set forth in SEQ ID NO: 10, 16, 24, 25, 26, 27, 28, or 29.

66. The method of paragraph 57, wherein the anteiso fatty acids are medium chain anteiso fatty acids.

67. The method of paragraph 57, wherein the anteiso fatty acids are not naturally produced in the cell.

68. Anteiso fatty acids produced by the method of paragraph 57.

69. An Escherichia coli cell that produces anteiso fatty acids.

70. The cell of paragraph 69, wherein the anteiso fatty acids are medium chain fatty acids.

71. The cell of paragraph 69, wherein the cell comprises a polynucleotide with at least 30 percent sequence identity to a sequence set forth in SEQ ID NO: 1, 4, 7, 13, 17, 18, 19, 20, 21, 22, or 23.

72. Anteiso fatty acids produced by the cell of paragraph 69.

73. A method of increasing anteiso fatty acids in a cell, comprising:

a. expressing in a cell one or more polynucleotide encoding an exogenous branched-chain amino acid aminotransferase, an exogenous branched-chain α-keto acid dehydrogenase (BCDH), and an exogenous 3-ketoacyl-ACP synthase;

b. culturing the cell under conditions such that anteiso fatty acids are produced.

74. The method of paragraph 73, wherein the method further includes expressing in the cell a polynucleotide encoding an exogenous thioesterase.

75. The method of paragraph 73, wherein the polynucleotide has at least 30 percent sequence identity to a sequence set forth in SEQ ID NO: 1, 4, 7, 13, 17, 18, 19, 20, 21, 22, or 23.

76. The method of paragraph 73, wherein the polynucleotide encodes a polypeptide having at least 40 percent sequence identity to a sequence set forth in SEQ ID NO: 10, 16, 24, 25, 26, 27, 28, or 29.

77. The method of paragraph 73, wherein the cell is an Escherichia coli cell and the polynucleotide has at least 65 percent sequence identity to a sequence set forth in SEQ ID NO: 1, 4, 7, 13, 17, 18, 19, 20, 21, 22, or 23.

78. The method of paragraph 76, wherein the polypeptide is substantially identical to a polypeptide having the sequence set forth in SEQ ID NO: 10, 16, 24, 25, 26, 27, 28, or 29.

79. The method of paragraph 73, wherein the anteiso fatty acids are medium chain anteiso fatty acids.

80. The method of paragraph 73, wherein the anteiso fatty acids are not naturally produced in the cell.

81. Anteiso fatty acids produced by the method of paragraph 73.

82. A method for making anteiso fatty acids, the method comprising culturing at least one cell comprising at least one exogenous polynucleotide that encodes at least one polypeptide that is capable of producing anteiso fatty acids from isoleucine under conditions such that anteiso fatty acids are produced.

83. The method of paragraph 82, wherein the cell is an Escherichia coli cell.

84. The method of paragraph 82, wherein the anteiso fatty acids are not naturally produced in the cell.

85. Anteiso fatty acids produced by the method of paragraph 82.

86. A cell comprising at least two exogenous polynucleotides, wherein the exogenous polynucleotides comprise nucleic acid sequences encoding polypeptides that catalyze at least two of the following reactions: a. conversion of leucine to 2-keto, 4-methylvalerate; b. conversion of valine to 2-keto 3-methylbutyrate; c. conversion of 2-keto, 4-methylvalerate to 3-methylbutyryl-CoA; d. conversion of 3-methylbutyryl-CoA to 3-methylbutyryl-ACP; e. conversion of 3-methylbutyryl-ACP to 5-methyl 3-ketohexanoyl-ACP; f. conversion of 2-keto 3-methylbutyrate to 2-methylpropionyl-CoA; g. conversion of 2-methylpropionyl-CoA to 2-methylpropionyl-ACP; h. conversion of 2-methylpropionyl-ACP to 4-methylvaleroyl-ACP; i. conversion of 3-methylbutyryl-CoA to 5-methyl 3-ketohexanoyl-ACP; j. conversion of 2-methylpropionyl-CoA to 4-methyl 3-ketovaleroyl-ACP; or k. conversion of acyl-ACP to iso fatty acids, and wherein the cell comprising the exogenous polynucleotides produces more iso fatty acids than an otherwise similar cell that does not comprise the exogenous polynucleotides.

87. The cell of paragraph 86, wherein the polynucleotides encode a branched-chain amino acid aminotransferase, a branched-chain α-keto acid dehydrogenase (BCDH), an acyl transferase, a 3-ketoacyl-ACP synthase, or a thioesterase.

88. The cell of paragraph 86, wherein the cell is an Escherichia cell.

89. The cell of paragraph 86, wherein the polynucleotides comprise nucleic acid sequences encoding polypeptides that catalyze 3, 4, 5, 6, 7, 8, 9, 10, or all of the reactions.

90. The cell of paragraph 86, wherein the polynucleotide has at least 30 percent sequence identity to a sequence set forth in SEQ ID NO: 1, 4, 7, 13, 17, 18, 19, 20, 21, 22, or 23.

91. The cell of paragraph 86, wherein the polypeptide has at least 40 percent sequence identity to a sequence set forth in SEQ ID NO: 10, 16, 24, 25, 26, 27, 28, or 29.

92. The cell of paragraph 86, wherein the cell is an Escherichia coli cell and the polynucleotide has at least 65 percent sequence identity to a sequence set forth in SEQ ID NO: 1, 4, 7, 13, 17, 18, 19, 20, 21, 22, or 23.

93. The cell of paragraph 86, wherein the polypeptide is substantially identical to a polypeptide having the sequence set forth in SEQ ID NO: 10, 16, 24, 25, 26, 27, 28, or 29.

94. The cell of paragraph 86, wherein the iso fatty acids are medium-chain iso fatty acids.

95. The cell of paragraph 86, wherein the iso fatty acids are not naturally produced in the cell.

96. Iso fatty acids produced by the cell of paragraph 86.

97. The cell of paragraph 86, wherein the polynucleotide encodes a fatty acid synthase gene from a Bacillus, a Streptomyces, or a Listeria.

98. The cell of paragraph 86, wherein the polynucleotide encodes a fatty acid synthase gene from an organism that naturally produces branched-chain fatty acids.

99. A method of increasing iso fatty acids in a bacterial cell, comprising:

a. expressing in a bacterial cell polynucleotides encoding at least two polypeptides, the polypeptides catalyzing at least two of the following reactions: i. conversion of leucine to 2-keto, 4-methylvalerate; ii. conversion of valine to 2-keto 3-methylbutyrate; iii. conversion of 2-keto, 4-methylvalerate to 3-methylbutyryl-CoA; iv. conversion of 3-methylbutyryl-CoA to 3-methylbutyryl-ACP; v. conversion of 3-methylbutyryl-ACP to 5-methyl 3-ketohexanoyl-ACP; vi. conversion of 2-keto 3-methylbutyrate to 2-methylpropionyl-CoA; vii. conversion of 2-methylpropionyl-CoA to 2-methylpropionyl-ACP; viii. conversion of 2-methylpropionyl-ACP to 4-methylvaleroyl-ACP; ix. conversion of 3-methylbutyryl-CoA to 5-methyl 3-ketohexanoyl-ACP; x. conversion of 2-methylpropionyl-CoA to 4-methyl 3-ketovaleroyl-ACP; and xi. conversion of acyl-ACP to iso fatty acids, and

b. culturing the bacterial cell under conditions that allow the cell to produce the polypeptides, such that iso fatty acids are produced.

100. A method of increasing accumulation of anteiso fatty acids in the culture medium by using a host strain unable to degrade fatty acids.

101. The host strain of paragraph 100, wherein the organism is E. coli.

102. The host strain of paragraph 100, wherein the organism is a fadD mutant of E. coli.

103. A method of increasing production of anteiso fatty acids in a cell, comprising: a. expressing in the cell a polynucleotide encoding a polypeptide having one of the following activities: citramalate synthase, isopropylmalate isomerase, and/or isopropylmalate dehydrogenase, and b. culturing the cell under conditions that allow the cell to produce the polypeptides, such that anteiso fatty acids are produced.

104. The method of paragraph 103, wherein the method includes expressing in the cell polynucleotides encoding polypeptides that have 2 or 3 of the activities.

105. A method for increasing production of anteiso fatty acids in a cell, comprising: a. expressing in the cell a polynucleotide encoding least one of ilvA, tdcB, ilvI, ilvH, ilvC, and/or ilvD, and b. culturing the cell under conditions that allow the cell to produce the polypeptides encoded by the polynucleotide, such that anteiso fatty acids are produced.

106. A method of increasing iso and/or anteiso fatty acid production in a cell, comprising: a. expressing in the cell a polynucleotide encoding a polypeptide having acetyl-CoA carboxylase activity, and b. culturing the cell under conditions that allow the cell to produce the polypeptides, such that iso and/or anteiso fatty acids are produced.

### BRIEF DESCRIPTION OF THE DRAWINGS

FIG. 1 is a diagram for anteiso and iso branched-chain fatty acid biosynthesis pathway.

FIG. 2 is a diagram for a threonine-dependent anteiso fatty acid biosynthesis pathway.

FIG. 3 is the DNA sequence for the amplified bkd operon (SEQ ID NO: 1).

FIG. 4 is the sequences for the bkd primers (SEQ ID NO: 2, 3).

FIG. 5 is the DNA sequence of the lpdV gene of bkd operon (SEQ ID NO: 4).

FIG. 6 is the sequences for the fabHA primers (SEQ ID NO: 5, 6, 8, 9).

FIG. 7 is the Bacillus subtilis fabHA DNA sequence (SEQ ID NO: 7).

FIG. 8 is the Bacillus subtilis FabHA amino acid sequence (SEQ ID NO: 10).

FIG. 9 is the sequences for the fabHB primers (SEQ ID NO: 11, 12, 14, and 15).

FIG. 10 is the sequence for the Bacillus subtilis fabHB DNA (SEQ ID NO: 13).

FIG. 11 is the Bacillus subtilis FabHB amino acid sequence (SEQ ID NO: 16).

FIG. 12 is the DNA sequence of the codon-optimized Mallard medium chain fatty acid thioesterase gene (SEQ ID NO: 17).

FIG. 13 is an alignment of the optimized open reading frame (ORF) (SEQ ID NO: 17) with the original Mallard medium chain fatty acid thioesterase sequence (SEQ ID NO: 18).

FIG. 14 is the DNA sequence of a codon-optimized rat mammary medium-chain fatty acid thioesterase gene (SEQ ID NO: 19).

FIG. 15 is an alignment of the optimized ORF (SEQ ID NO: 19) with the original rat mammary medium-chain fatty acid thioesterase (SEQ ID NO: 20).

FIG. 16 is a graph showing the effect of isoleucine supplementation on anteiso fatty acid production.

FIG. 17 is a diagram of a threonine-independent anteiso fatty acid synthesis pathway.

FIG. 18 is the DNA sequence of bkdAA gene of bkd operon (SEQ ID NO: 21)

FIG. 19 is the DNA sequence of bkdAB gene of bkd operon (SEQ ID NO: 22)

FIG. 20 is the DNA sequence of bkdB gene of bkd operon (SEQ ID NO: 23)

FIG. 21 is the protein sequence of lpdV gene of bkd operon (SEQ ID NO: 24)

FIG. 22 is the protein sequence of bkdAA gene of bkd operon (SEQ ID NO: 25)

FIG. 23 is the protein sequence of bkdAB gene of bkd operon (SEQ ID NO: 26)

FIG. 24 is the protein sequence of bkdB gene of bkd operon (SEQ ID NO: 27)

FIG. 25 is the protein sequence of Mallard medium-chain fatty acid thioesterase (SEQ ID NO: 28)

FIG. 26 is the protein sequence of rat mammary medium-chain fatty acid thioesterase (SEQ ID NO: 29)

FIG. 27 is a bar graph illustrating C15 anteiso fatty acid production (fraction of a-C15 anteiso fatty acids in the total pool of synthesized fatty acids; y-axis) in E. coli strains K27-Z1 (parental strain), K27-Z1 (Bs bkd Bs fabH), K27-Z1 (Bs bkd Bs fabH Ec tcdB), and K27-Z1 (Bs bkd Bs fabH Ec tdcB Ec ilvIH(G14D)) (x-axis). Cultures were prepared in triplicate, with standard deviation of fatty acid measurements indicated by error bars.

FIG. 28 is a bar graph illustrating C15 and C17 anteiso fatty acid production in E. coli K27-Z1 derivative strains expressing different AHAS genes. The K27-Z1 derivative strains x-axis) comprised the following plasmids and genes: (i) pTrcHisA and pZA31MCS (Vector Control); (ii) Bs bkd Bs fabHA pTrcHisA; (iii) Bs bkd fabHA Ec tdcB; (iv) Bs bkd fabHA Ec tdcB Ec ilvIH; (v) Bs bkd fabHA Ec tdcB Ec ilvIH(G14D); and (vi) Bs bkd Bs fabHA Ec tdcB Bs ilvBH. The peak area from gas chromatography analysis is represented on the y-axis. One biological replicate is represented with duplicate fatty acid analysis.

FIG. 29 is a bar graph illustrating C15 anteiso fatty acid production in E. coli BL21 Star (DE3) derivatives.

FIG. 30 is a bar graph illustrating C15 and C17 anteiso fatty acid production in the following E. coli derivative strains: K27-Z1 (pTrcHisA pZA31 MCS (vector control)), K27-Z1 (Bs bkd Bs fabHA), K27-Z1 (Bs bkd Bs fabHA Ec tdcB), and K27-Z1 (Bs bkd Bs fabHA Ec tdcB Ec ilvGM). One biological replicate was tested with duplicate fatty acid analysis; standard deviations are indicated by error bars.

FIG. 31 is a bar graph illustrating C15 and C17 anteiso fatty acid production in E. coli K27-Z1 derivatives designed to produce the indicated recombinant proteins: Bkd-FabHA, Bkd-FabHA-CimA-LeuBCD, Bkd-FabHA-CimA-LeuBCD-IlvIH, Bkd-FabHA-CimA-LeuBCD-IlvIH(G14D), Bkd-FabHA-CimA-LeuBCD-IlvBH, and Bkd-FabHA-CimA-LeuBCD-IlvGM.

FIG. 32 is a bar graph illustrating C13, C15, and C17 anteiso fatty acid production in E. coli K27-Z1 (Bs bkd Bs fabH) and E. coli K27-Z1 (Bs bkd Bs fabHA Ec ‘tesA).

FIG. 33 is a bar graph illustrating C13, C15, and C17 anteiso fatty acid production in E. coli BL21 Star (DE3) (Bs bkd Bs fabHA) and BL21 Star (DE3) (Bs bkd Bs fabHA Ec ‘tesA).

FIG. 34 is a bar graph illustrating C15 and C17 anteiso fatty acid production in E. coli cultured in the presence and absence of thiamine. The E. coli derivatives were designed to produce the indicated recombinant proteins. Duplicate samples are indicated by “#2.” The presence of thiamine in the culture medium improved anteiso fatty acid production.

FIG. 35 is a bar graph illustrating C15 and C17 anteiso fatty acid production in an E. coli ilvE deletion strain (Bs bkd Bs fabHA Ec tdcB Ec ilvIH(G14D)) and a control ilvE deletion strain (pZA31 MCS pTrcHisA).

FIG. 36 is a bar graph illustrating anteiso and iso branched-chain fatty acid production in E. coli BW25113 derivatives harboring polynucleotides encoding Listeria monocytogenes FabH and B. subtilis Bkd.

### DETAILED DESCRIPTION OF THE INVENTION

The invention relates to biologically produced anteiso and/or iso branched-chain fatty acids and improved biological production of such anteiso and/or iso branched-chain fatty acids. This improved biological production can, in certain embodiments, provide higher yields of anteiso and/or iso branched-chain fatty acids. In addition, or alternatively, the invention provides the ability to tailor the chain length of the anteiso and/or iso branched-chain fatty acids to a desired chain length.

As used herein, “amplify,” “amplified,” or “amplification” refers to any process or protocol for copying a polynucleotide sequence into a larger number of polynucleotide molecules, e.g., by reverse transcription, polymerase chain reaction, and ligase chain reaction.

As used herein, an “antisense sequence” refers to a sequence that specifically hybridizes with a second polynucleotide sequence. For instance, an antisense sequence is a DNA sequence that is inverted relative to its normal orientation for transcription. Antisense sequences can express an RNA transcript that is complementary to a target mRNA molecule expressed within the host cell (e.g., it can hybridize to target mRNA molecule through Watson-Crick base pairing).

As used herein, “cDNA” refers to a DNA that is complementary or identical to an mRNA, in either single stranded or double stranded form.

As used herein, “complementary” refers to a polynucleotide that base pairs with a second polynucleotide. Put another way, “complementary” describes the relationship between two single-stranded nucleic acid sequences that anneal by base-pairing. For example, a polynucleotide having the sequence 5′-GTCCGA-3′ is complementary to a polynucleotide with the sequence 5′-TCGGAC-3′.

As used herein, a “conservative substitution” refers to the substitution in a polypeptide of an amino acid with a functionally similar amino acid. Put another way, a conservative substitution involves replacement of an amino acid residue with an amino acid residue having a similar side chain. Families of amino acid residues having similar side chains have been defined within the art, and include amino acids with basic side chains (e.g., lysine, arginine, and histidine), acidic side chains (e.g., aspartic acid and glutamic acid), uncharged polar side chains (e.g., glycine, asparagine, glutamine, serine, threonine, tyrosine, and cysteine), nonpolar side chains (e.g., alanine, valine, leucine, isoleucine, proline, phenylalanine, methionine, and tryptophan), beta-branched side chains (e.g., threonine, valine, and isoleucine) and aromatic side chains (e.g., tyrosine, phenylalanine, tryptophan, and histidine).

As used herein, “encoding” refers to the inherent property of nucleotides to serve as templates for synthesis of other polymers and macromolecules. Unless otherwise specified, a “nucleotide sequence encoding an amino acid sequence” includes all nucleotide sequences that are degenerate versions of each other and that encode the same amino acid sequence.

As used herein, “endogenous” refers to polynucleotides, polypeptides, or other compounds that are expressed naturally or originate within an organism or cell. That is, endogenous polynucleotides, polypeptides, or other compounds are not exogenous. For instance, an “endogenous” polynucleotide or peptide is present in the cell when the cell was originally isolated from nature.

As used herein, “expression vector” refers to a vector comprising a recombinant polynucleotide comprising expression control sequences operatively linked to a nucleotide sequence to be expressed. For example, suitable expression vectors can be an autonomously replicating plasmid or integrated into the chromosome. An expression vector also can be a viral-based vector.

As used herein, “exogenous” refers to any polynucleotide or polypeptide that is not naturally expressed in the particular cell or organism where expression is desired. Exogenous polynucleotides, polypeptides, or other compounds are not endogenous.

As used herein, “hybridization” includes any process by which a strand of a nucleic acid joins with a complementary nucleic acid strand through base-pairing. Thus, the term refers to the ability of the complement of the target sequence to bind to a test (i.e., target) sequence, or vice-versa.

As used herein, “hybridization conditions” are typically classified by degree of “stringency” of the conditions under which hybridization is measured. The degree of stringency can be based, for example, on the melting temperature (Tₘ) of the nucleic acid binding complex or probe. For example, “maximum stringency” typically occurs at about Tₘ-5° C. (5° below the Tₘ of the probe); “high stringency” at about 5-10° below the Tₘ; “intermediate stringency” at about 10-20° below the Tₘ of the probe; and “low stringency” at about 20-25° below the Tₘ. Alternatively, or in addition, hybridization conditions can be based upon the salt or ionic strength conditions of hybridization and/or one or more stringency washes. For example, 6×SSC=very low stringency; 3×SSC=low to medium stringency; 1×SSC=medium stringency; and 0.5×SSC=high stringency. Functionally, maximum stringency conditions may be used to identify nucleic acid sequences having strict (i.e., about 100%) identity or near-strict identity with the hybridization probe; while high stringency conditions are used to identify nucleic acid sequences having about 80% or more sequence identity with the probe.

As used herein, “identical” or percent “identity,” in the context of two or more polynucleotide or polypeptide sequences, refers to two or more sequences that are the same or have a specified percentage of nucleotides or amino acid residues that are the same, when compared and aligned for maximum correspondence, as measured using sequence comparison algorithms or by visual inspection.

As used herein, “long-chain fatty acids” refers to fatty acids with aliphatic tails longer than 14 carbons.

As used herein, “medium-chain fatty acids” refers to fatty acids with aliphatic tails between 6 and 14 carbons. In certain embodiments, the medium-chain fatty acids can have from 11 to 13 carbons.

As used herein, “naturally-occurring” refers to an object that can be found in nature. For example, a polypeptide or polynucleotide sequence that is present in an organism (including viruses) that can be isolated from a source in nature and which has not been intentionally modified by man in the laboratory is naturally-occurring.

As used herein, “operably linked,” when describing the relationship between two DNA regions or two polypeptide regions, means that the regions are functionally related to each other. For example, a promoter is operably linked to a coding sequence if it controls the transcription of the sequence; a ribosome binding site is operably linked to a coding sequence if it is positioned so as to permit translation; and a sequence is operably linked to a peptide if it functions as a signal sequence, such as by participating in the secretion of the mature form of the protein.

As used herein, “overexpression” refers to expression of a polynucleotide to produce a product (e.g., a polypeptide or RNA) at a higher level than the polynucleotide is normally expressed in the host cell. An overexpressed polynucleotide is generally a polynucleotide native to the host cell, the product of which is generated in a greater amount than that normally found in the host cell. Overexpression is achieved by, for instance and without limitation, operably linking the polynucleotide to a different promoter than the polynucleotide's native promoter or introducing additional copies of the polynucleotide into the host cell.

As used herein, “polynucleotide” refers to a polymer composed of nucleotides. The polynucleotide may be in the form of a separate fragment or as a component of a larger nucleotide sequence construct, which has been derived from a nucleotide sequence isolated at least once in a quantity or concentration enabling identification, manipulation, and recovery of the sequence and its component nucleotide sequences by standard molecular biology methods, for example, using a cloning vector. When a nucleotide sequence is represented by a DNA sequence (i.e., A, T, G, C), this also includes an RNA sequence (i.e., A, U, G, C) in which “U” replaces “T.” Put another way, “polynucleotide” refers to a polymer of nucleotides removed from other nucleotides (a separate fragment or entity) or can be a component or element of a larger nucleotide construct, such as an expression vector or a polycistronic sequence. Polynucleotides include DNA, RNA and cDNA sequences.

As used herein, “polypeptide” refers to a polymer composed of amino acid residues which may or may not contain modifications such as phosphates and formyl groups.

As used herein, “recombinant expression vector” refers to a DNA construct used to express a polynucleotide that, e.g., encodes a desired polypeptide. A recombinant expression vector can include, for example, a transcriptional subunit comprising (i) an assembly of genetic elements having a regulatory role in gene expression, for example, promoters and enhancers, (ii) a structural or coding sequence which is transcribed into mRNA and translated into protein, and (iii) appropriate transcription and translation initiation and termination sequences. Recombinant expression vectors are constructed in any suitable manner. The nature of the vector is not critical, and any vector may be used, including plasmid, virus, bacteriophage, and transposon. Possible vectors for use in the invention include, but are not limited to, chromosomal, nonchromosomal and synthetic DNA sequences, e.g., bacterial plasmids; phage DNA; yeast plasmids; and vectors derived from combinations of plasmids and phage DNA, DNA from viruses such as vaccinia, adenovirus, fowl pox, baculovirus, SV40, and pseudorabies.

As used herein, “primer” refers to a polynucleotide that is capable of specifically hybridizing to a designated polynucleotide template and providing a point of initiation for synthesis of a complementary polynucleotide when the polynucleotide primer is placed under conditions in which synthesis is induced. As used herein, “recombinant polynucleotide” refers to a polynucleotide having sequences that are not naturally joined together. A recombinant polynucleotide may be included in a suitable vector, and the vector can be used to transform a suitable host cell. A host cell that comprises the recombinant polynucleotide is referred to as a “recombinant host cell.” The polynucleotide is then expressed in the recombinant host cell to produce, e.g., a “recombinant polypeptide.”

As used herein, “specific hybridization” refers to the binding, duplexing, or hybridizing of a polynucleotide preferentially to a particular nucleotide sequence under stringent conditions.

As used herein, “stringent conditions” refers to conditions under which a probe will hybridize preferentially to its target subsequence, and to a lesser extent to, or not at all to, other sequences.

As used herein, “short chain fatty acids” refers to fatty acids having aliphatic tails with fewer than 6 carbons.

As used herein, “substantially homologous” or “substantially identical” in the context of two nucleic acids or polypeptides, generally refers to two or more sequences or subsequences that have at least 40%, 60%, 80%, 90%, 95%, 96%, 97%, 98% or 99% nucleotide or amino acid residue identity, when compared and aligned for maximum correspondence, as measured using sequence comparison algorithms or by visual inspection. The substantial identity can exist over any suitable region of the sequences, such as, for example, a region that is at least about 50 residues in length, a region that is at least about 100 residues, or a region that is at least about 150 residues. In certain embodiments, the sequences are substantially identical over the entire length of either or both comparison biopolymers.

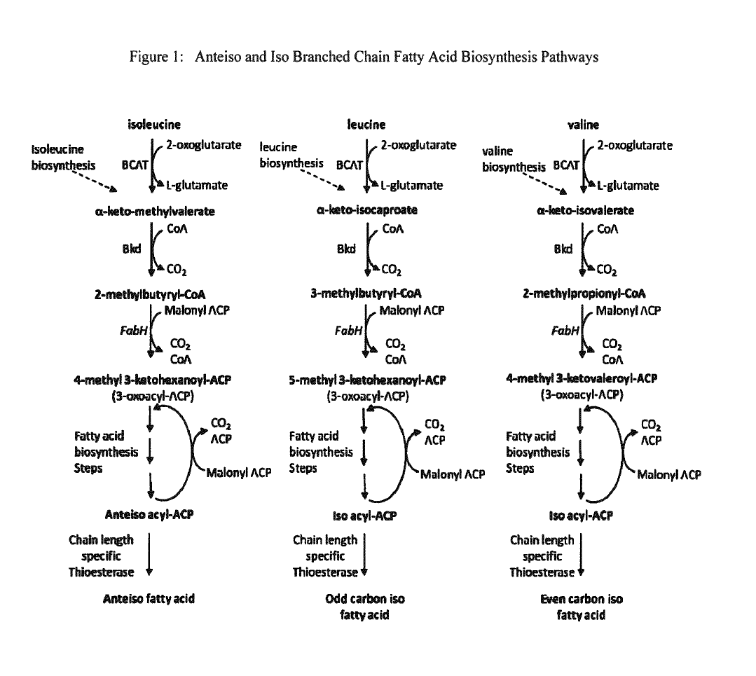

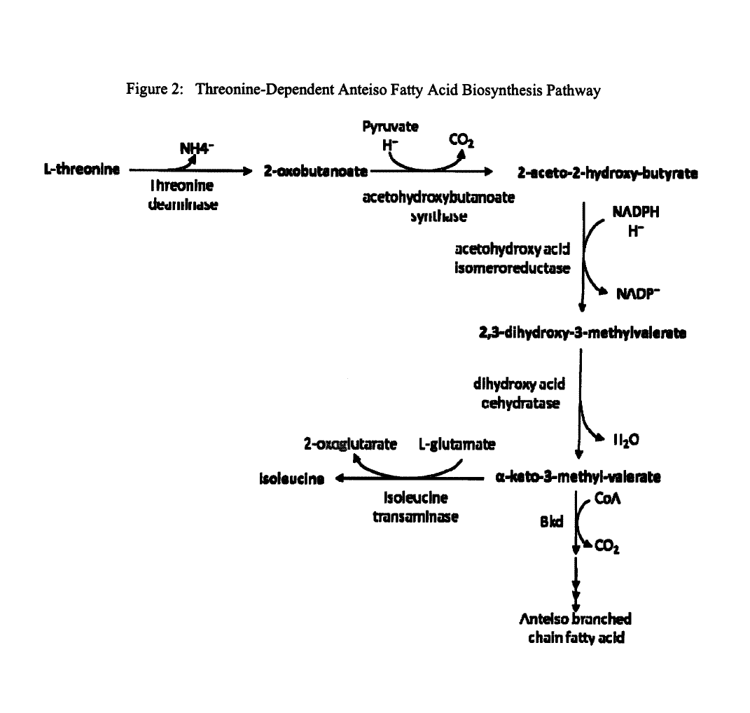

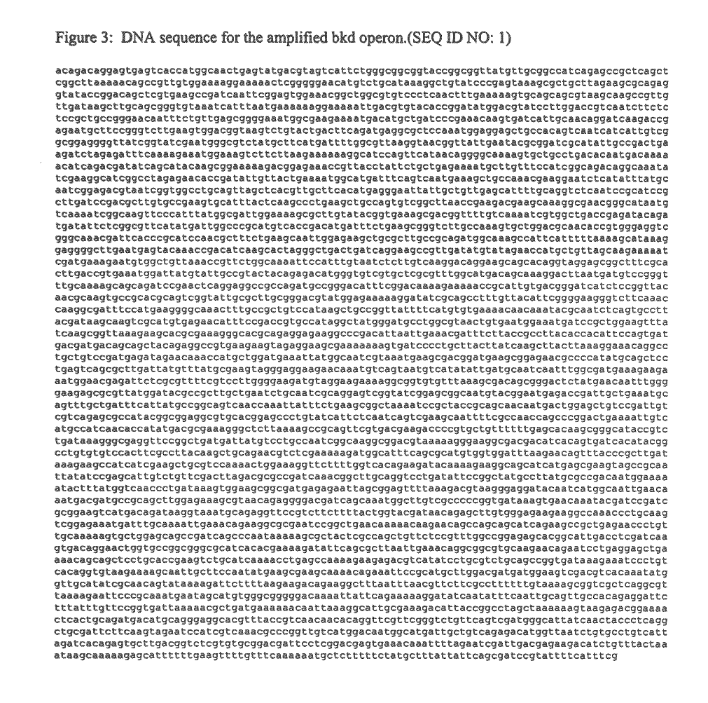

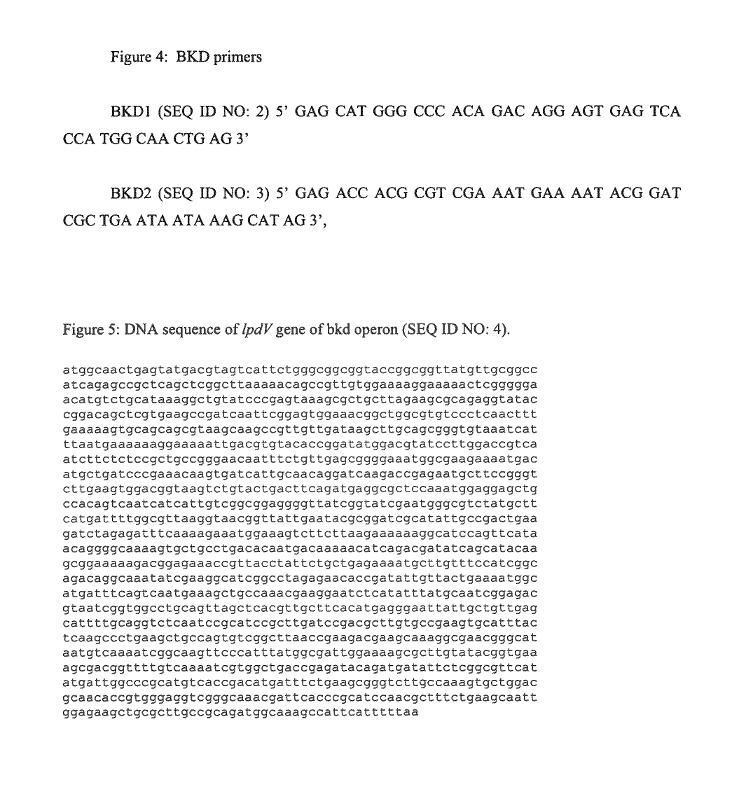

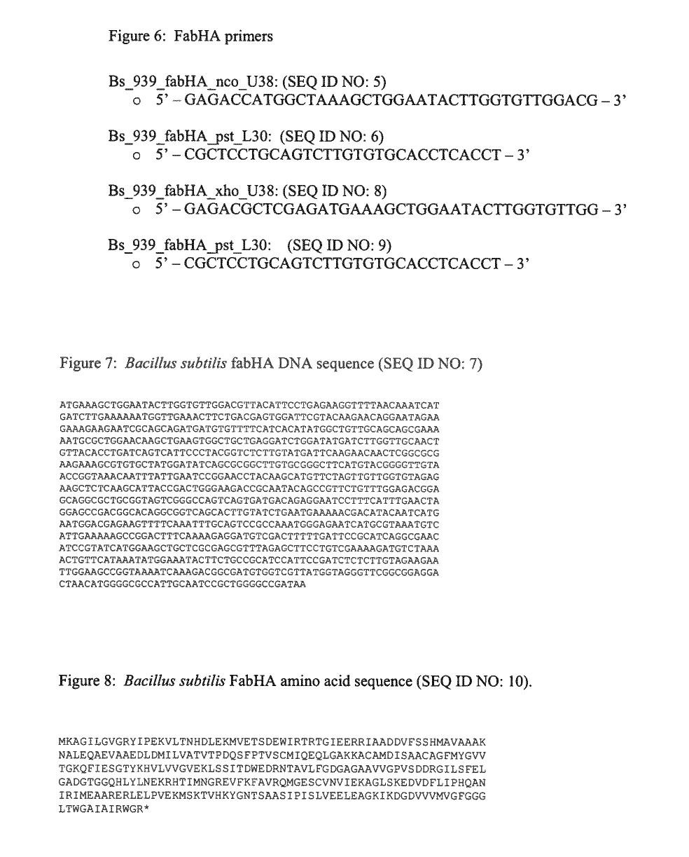

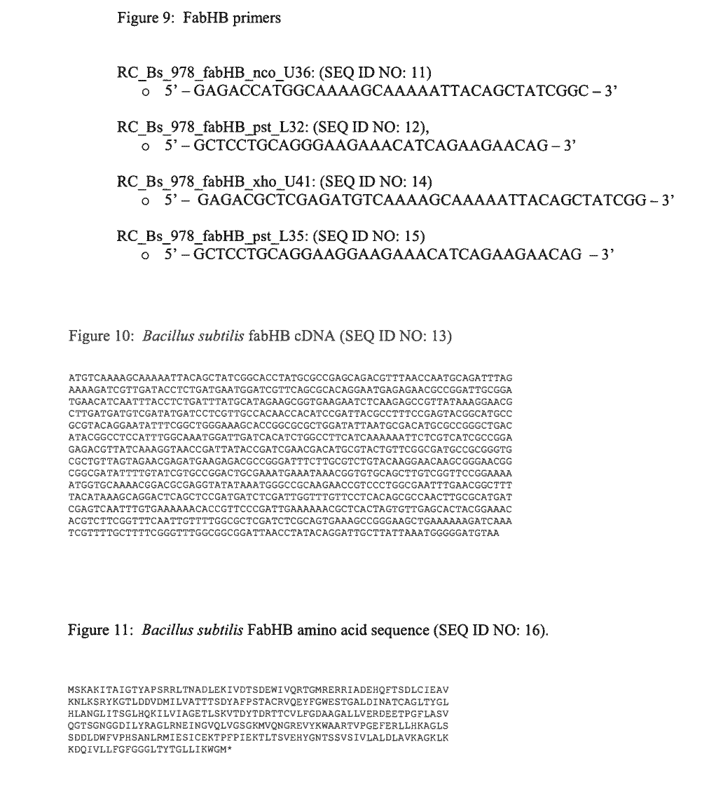

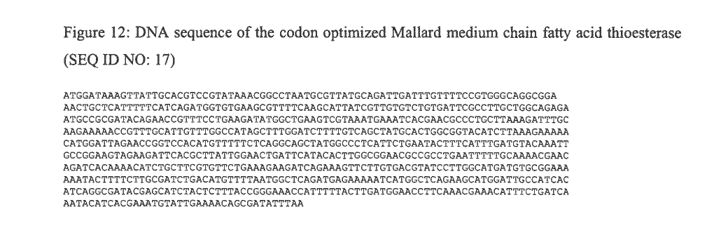

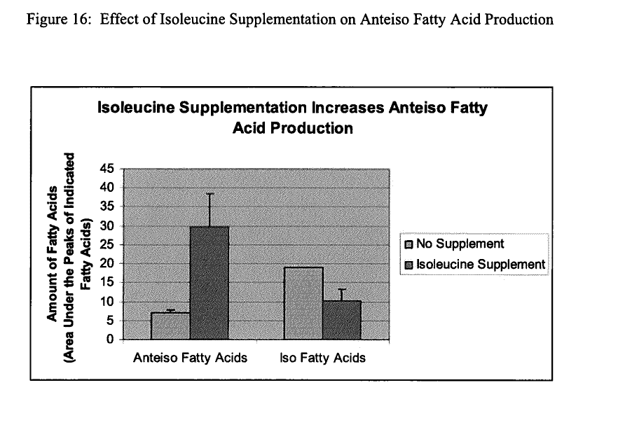

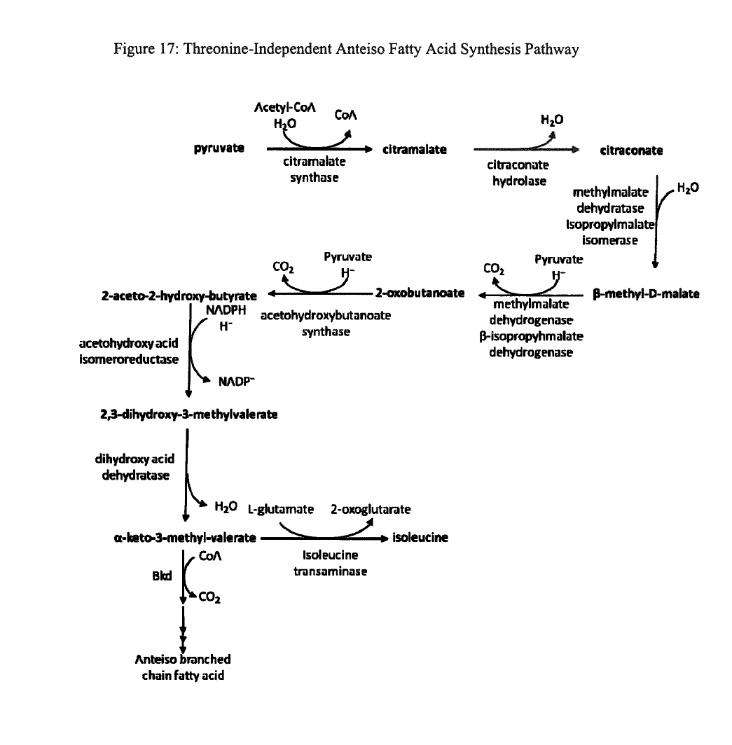

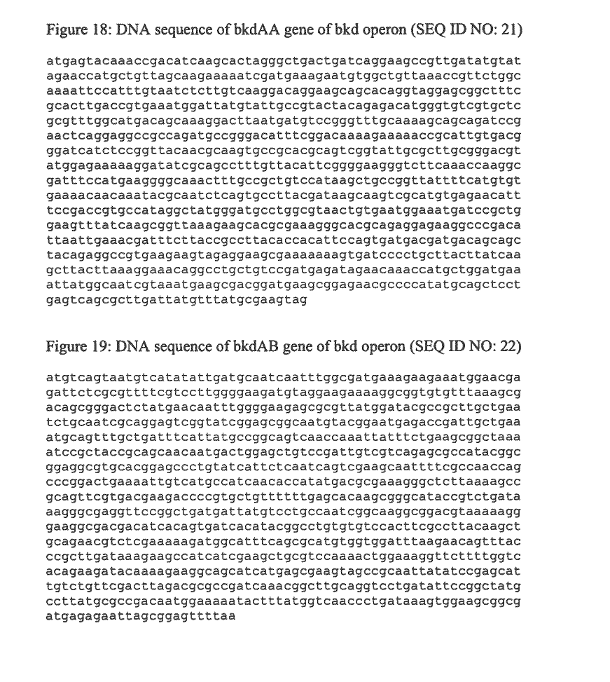

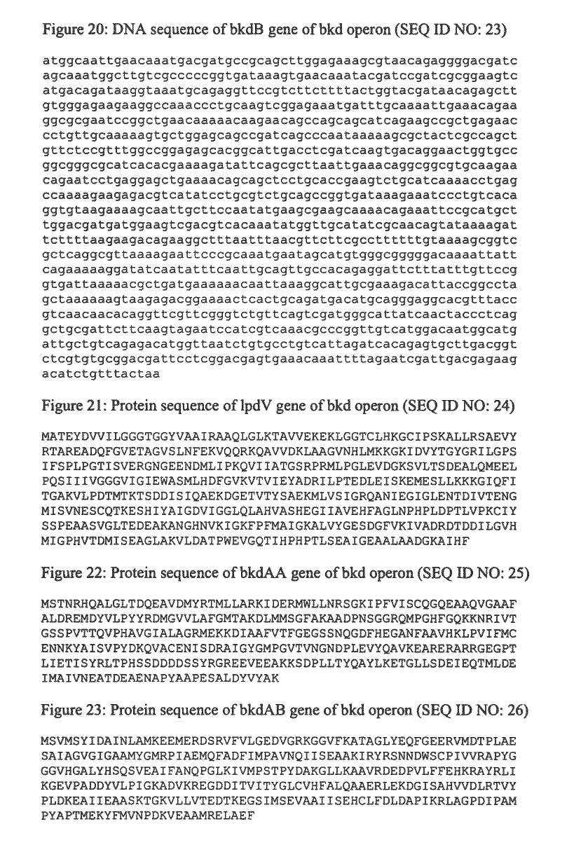

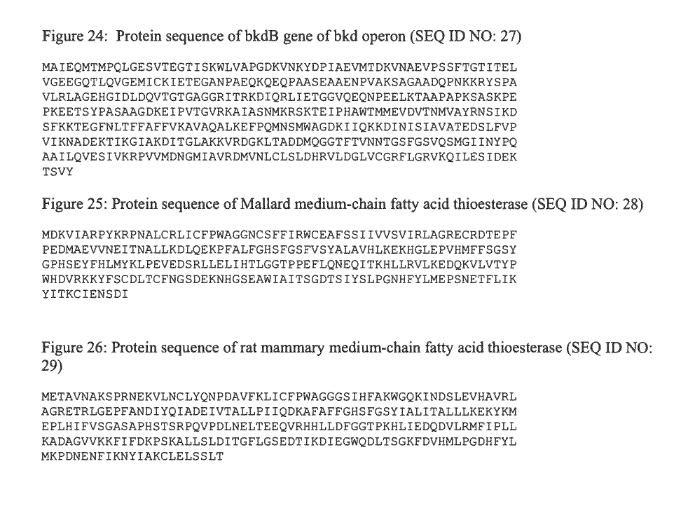

In one aspect, the invention relates to a novel method of producing anteiso and/or iso branched chain fatty acids (or products derived from anteiso and/or iso branched-chain fatty acids) using bacteria. In one aspect, the method features incorporating one or more exogenous polynucleotides that increase production of anteiso or iso fatty acid in a suitable cell, such as, for example, by transfecting or transforming the cell with the polynucleotide(s). Alternatively or in addition, the method comprises overexpressing one or more polynucleotides to increase production of anteiso or iso fatty acid within the host cell. Exemplary metabolic pathways for producing anteiso and iso fatty acid in a host cell are illustrated in FIGS. 1, 2, and 17. FIG. 1 illustrates metabolic pathways for producing (1) anteiso fatty acid via a pathway that includes conversion of isoleucine to 2-keto 3-methylvalerate, (2) odd total carbon iso-branched-chain fatty acids via a pathway that includes conversion of leucine to 2-keto-isocaproate (also referred to as 2-keto, 4-methylvalerate), and (3) even numbered total carbon iso-branched-chain fatty acids via a pathway that includes conversion of valine to 2-keto-isovalerate (also referred to as 2-keto 3-methylbutyrate). In certain embodiments, driving the carbon flow to the branched 2-keto acid precursor results in increased production of the corresponding branched-chain fatty acid. For example, 1) increasing the carbon flow to the isoleucine pathway results in increased production of anteiso fatty acids; 2) increasing the carbon flow to the leucine pathway results in increased production of iso branched-chain fatty acid with an odd number of carbons; and/or 3) increasing the carbon flow to the valine pathway results in increased production of the iso branched-chain fatty acid with an even number of carbons. FIGS. 2 and 17 illustrate pathways for generating isoleucine and/or 2-keto 3-methylvalerate from threonine or pyruvate, respectively. Increasing carbon flow through the threonine and/or pyruvate pathways enhance the production of anteiso branched-chain fatty acid in a recombinant host cell.

In one aspect, the invention provides a method for producing anteiso fatty acid. The method comprises culturing a cell comprising at least one exogenous or overexpressed polynucleotide comprising a nucleic acid sequence encoding a polypeptide that catalyzes at least one of the following reactions: (aa) conversion of pyruvate to citramalate; (bb) conversion of citramalate to citraconate; (cc) conversion of citraconate to β-methyl-D-malate; (dd) conversion of β-methyl-D-malate to 2-oxobutanoate; or (ee) conversion of threonine to 2-oxobutanoate. Optionally, the cell further comprises at least one exogenous or overexpressed polynucleotide comprising a nucleic acid sequence encoding a polypeptide that catalyzes at least one of the following reactions: (ff) conversion of 2-oxobutanoate to 2-aceto-2-hydroxy-butyrate, (gg) conversion of 2-aceto-2-hydroxy-butyrate to 2,3-dihydroxy-3-methylvalerate, or (hh) conversion of 2,3-dihydroxy-3-methylvalerate to α-keto-3-methylvalerate. In some embodiments, the cell comprises exogenous or overexpressed polynucleotides encoding polypeptides that catalyze 3, 4, 5, 6, 7, or all of the reactions (aa)-(hh). The cell is cultured under conditions allowing expression of the polynucleotide(s) and production of anteiso fatty acid. The invention is predicated, at least in part, on the observation that host cells comprising the genetic modifications described herein produce more anteiso fatty acids than an otherwise similar cell that does not comprise the polynucleotide(s). Metabolic pathways and genetic modifications for increasing anteiso and iso fatty acid production in a cell are further described below.

One method for increasing carbon flow to the isoleucine pathway comprises upregulating production of 2-keto 3-methylvalerate through the threonine-dependent pathway of FIG. 2. Threonine can be produced at high levels in, e.g., E. coli (Lee et al., Molecular Systems Biology 3: 149 (2007)) and, through a series of steps shown in FIG. 2, isoleucine is produced from threonine via 2-keto 3-methylvalerate as an intermediate. As illustrated in FIG. 2, the threonine-dependent pathway entails conversion of threonine to 2-oxobutanoate by, e.g., threonine deaminase; conversion of 2-oxobutanoate to 2-aceto2-hydroxy-butyrate by, e.g., acetohydroxy acid synthase (AHAS) (also known as acetohydroxybutanoate synthase); conversion of 2-aceto 2-hydroxy-butyrate to 2,3-dihydroxy-3-methylvalerate by, e.g., acetohydroxy acid isomeroreductase; and conversion of 2,3-dihydroxy-3-methylvalerate to 2-keto-3-methyl-valerate by, e.g., dihydroxy acid dehydratase. The pathway is optimized for carbon flow to 2-keto-3-methyl-valerate and ultimately to the anteiso fatty acid by expressing exogenous polynucleotides or overexpressing endogenous polynucleotides encoding any one or more of the activities described above. For example, the pathway is optimized for carbon flow to 2-keto 3-methyl-valerate by overexpressing ilvA, tdcB, ilvI, ilvH, ilvC and/or ilvD. IlvA and TdcB are threonine deaminases. IlvC is an acetohydroxy acid isomeroreductase (also known as ketol-acid reductoisomerase), and IlvD is a dihydroxy acid dehydratase. IlvI and IlvH are two subunits that form AHAS, which catalyzes the formation of 2-acetolactate from pyruvate for valine and leucine synthesis, or the formation of 2-aceto-2-hydroxybutyrate from 2-oxobutanoate and pyruvate for isoleucine biosynthesis (see FIG. 1). The two AHAS reactions are irreversible and committed steps toward the synthesis of two different sets of branched-chain amino acids. In certain embodiments, for example, deletion of the AHAS I (ilvBN) and/or overproduction of AHAS II (ilvGM) and/or AHAS III (ilvIH) minimize production of iso fatty acid derived from leucine or valine. IlvBH also is an AHAS suitable for use in the context of the invention.

Thus, in one aspect, the cell of the invention comprises an exogenous or overexpressed polynucleotide encoding a polypeptide that catalyzes the conversion of threonine to 2-oxobutanoate (e.g., a threonine deaminase) and an exogenous or overexpressed polynucleotide encoding a polypeptide that catalyzes the conversion of 2-oxobutanoate to 2-aceto 2-hydroxy-butyrate (e.g., an AHAS).

In certain embodiments, cells or organisms of the invention are engineered to accumulate anteiso fatty acids under nitrogen-limiting conditions and to utilize a threonine-independent isoleucine synthesis pathway, such as the pyruvate pathway shown in FIG. 17. As illustrated in FIG. 17, pyruvate is combined with acetyl-CoA to produce citramalate by, e.g., citramalate synthase. An exemplary citramalate synthase is CimA, such as CimA derived from M. jannaschii. Citramalate is then converted to citraconate by, e.g., a citraconate hydrolase (also known as isopropylmalate or citramalate isomerase), an example of which is encoded by leuCD. Citraconate is converted to β-methyl-D-malate by, e.g., an isopropylmalate isomerase (such as LeuCD), and the resulting β-methyl-D-malate is converted to 2-oxobutanoate (also referred to as α-ketobutyrate) by, e.g., isopropylmalate dehydrogenase (such as LeuB). The pyruvate pathway converges with the threonine pathway of FIG. 2, as 2-oxobutanoate is converted to 2-aceto-2-hydroxy-butyrate by, e.g., AHAS; 2-aceto 2-hydroxy-butyrate is converted to 2,3-dihydroxy-3-methylvalerate by, e.g., acetohydroxy acid isomeroreductase; and 2,3-dihydroxy-3-methylvalerate is converted to 2-keto 3-methyl-valerate by, e.g., dihydroxy acid dehydratase. The pathway is optimized for carbon flow to 2-keto 3-methyl-valerate and ultimately to the anteiso fatty acid by expressing exogenous polynucleotides or overexpressing endogenous polynucleotides encoding any one or more of the activities described above. For example, the pathway is optimized for carbon flow to 2-keto 3-methyl-valerate by overexpressing or expressing exogenous cimA, leuCD, leuB, ilvI, ilvH, ilvC ilvG, ilvM, and/or ilvD.

Thus, in one aspect, the cell of the invention comprises exogenous or overexpressed polynucleotides encoding polypeptides that catalyze the conversion of pyruvate to citramalate, the conversion of citramalate to citraconate, the conversion of citraconate to β-methyl-D-malate, and the conversion of 2-oxobutanoate to 2-aceto-2-hydroxy-butyrate. For example, in one embodiment, the cell comprises an exogenous or overexpressed polynucleotide comprising a nucleic acid sequence encoding a citramalate synthase, an exogenous or overexpressed polynucleotide comprising a nucleic acid sequence encoding an isopropylmalate isomerase, an exogenous or overexpressed polynucleotide comprising a nucleic acid sequence encoding an isopropylmalate dehydrogenase, an exogenous or overexpressed polynucleotide comprising a nucleic acid sequence encoding a AHAS, or a combination thereof, including a combination of polynucleotides encoding all four polypeptides.

In one aspect, the host cell is modified to express an exogenous polynucleotide or overexpress a native polynucleotide encoding one or more enzyme activities that mediate downstream reactions yielding anteiso or iso fatty acid from isoleucine, leucine, or valine. For example, as shown in FIG. 1, in certain embodiments, cells are modified to produce anteiso fatty acids via a pathway that includes conversion of isoleucine to 2-keto 3-methylvalerate by a branched-chain amino acid aminotransferase (BCAT). Alternatively, the 2-keto 3-methylvalerate is introduced into isoleucine biosynthesis pathway without first being converted to isoleucine. The 2-keto 3-methylvalerate is then converted to 2-methylbutyryl-CoA by, e.g., a branched-chain α-keto acid dehydrogenase (BCDH), such as a BCDH encoded by bkd. The 2-methylbutyryl-CoA is condensed with a malonyl-ACP by, e.g., a 3-ketoacyl-ACP synthase, and the subsequent incorporation of malonyl-ACP is processed via fatty acid biosynthesis to anteiso acyl-ACP. Acyl-ACP is then converted to anteiso fatty acids via a thioesterase.

In certain embodiments, odd total carbon iso-branched-chain fatty acids are produced via a pathway that includes conversion of leucine to 2-keto-isocaproate (also referred to as 2-keto, 4-methylvalerate) by, e.g., a BCAT. If desired, 2-keto-isocaproate is introduced into the leucine biosynthesis pathway without first being converted to leucine. The 2-keto-isocaproate is then converted to isovaleryl-CoA (also referred to as 3-methylbutyryl-CoA) by, e.g., a BCDH, such as a BCDH encoded by bkd. The isovaleryl-CoA is condensed with a malonyl-ACP by, e.g., a 3-ketoacyl-ACP synthase, and the subsequent incorporation of malonyl-ACP is processed via fatty acid biosynthesis to iso acyl-ACP. Iso acyl-ACP is then converted to iso fatty acids via a thioesterase.

Furthermore, in certain embodiments, even numbered total carbon iso-branched-chain fatty acids are produced via a pathway that includes conversion of valine to 2-keto-isovalerate (also referred to as 2-keto 3-methylbutyrate) by a BCAT. Optionally, 2-keto-isovalerate is introduced into the valine biosynthesis pathway without first being converted to valine. The 2-keto-isovalerate is then converted to isobutyryl-CoA by, e.g., a BCDH, such as a BCDH encoded by bkd. The isobutyryl-CoA is condensed with a malonyl-ACP by, e.g., a 3-ketoacyl-ACP synthase, and the subsequent incorporation of malonyl-ACP is processed via fatty acid biosynthesis to iso acyl-ACP. Iso acyl-ACP can then be converted to iso fatty acids via a thioesterase.

Thus, in some embodiments, the host cell comprises an exogenous or overexpressed polynucleotide encoding a BCDH or a biologically active fragment or variant thereof. Alternatively or in addition, the cell comprises an exogenous or overexpressed polynucleotide encoding a BCAT and/or an exogenous or overexpressed polynucleotide encoding an acyl transferase. Alternatively or in addition, the cell comprises an exogenous or overexpressed polynucleotide encoding a 3-ketoacyl-ACP synthase that uses anteiso and/or iso branched-CoA primers as substrates into a suitable cell. In addition or alternatively, the cell comprises an exogenous or overexpressed polynucleotide comprising a nucleic acid sequence encoding an enoyl-ACP reductase. In addition, or alternatively, the cell comprises an exogenous or overexpressed polynucleotide encoding a thioesterase. Depending on the activity and substrate specificity of the thioesterase, such recombinant cells can produce anteiso and/or iso branched-chain fatty acids having a desired chain length. For example, in some embodiments, the host cell preferentially generates long chain fatty acid, medium-length chain fatty acid, or a desired combination thereof (e.g., 60%, 70%, 80%, 85%, 90%, 95% or more of the fatty acid comprises the desired number of carbons). Combinations of any of the enzymes described herein also is contemplated, such as, for example, a cell comprising exogenous or overexpressed polynucleotides encoding BCDH, 3-ketoacyl-ACP synthase, and thioesterase (such as TesA). Indeed, the invention contemplates a cell engineered to increase carbon flow through the threonine-dependent and/or threonine-independent pathways as described above and further comprising exogenous or overexpressed polynucleotides that augment carbon flow through the isoleucine, leucine, and/or valine pathways illustrated in FIG. 1. Optionally, one or more of the exogenous or overexpressed polynucleotides comprise a nucleic acid sequence having at least 90 percent identity to the nucleic acid sequences set forth in SEQ ID NOs: 1, 4, 7, 13, 17-23, 68, 77, or 78.

In addition, or alternatively, in certain embodiments, production of anteiso and/or iso branched-chain fatty acids is enhanced by modifying cells to increase acetyl-CoA carboxylase activity. For example, production of one or more of the enzyme subunits is increased by, e.g., increasing the amount of available biotin or by increasing the activity or amount of the biotin-protein ligase, BirA. Upregulating thiamine levels in a host cell by, for instance, augmenting thiamine synthase production, also is contemplated herein to further enhance branched-chain fatty acid synthesis.

In some embodiments of the invention, the cell is engineered to express one or more exogenous polynucleotides encoding one or more of the enzyme activities described herein and/or is engineered to overexpress one or more endogenous polynucleotides encoding one or more of the enzyme activities described herein. Different organisms manufacture fatty acids using different pathways, and endogenous fatty acid synthesis reactions can leech resources away from branched-chain fatty acid synthesis. Thus, in certain embodiments, native enzyme activity is attenuated to enhance branched-chain fatty acid synthesis. For example, in E. coli, which does not naturally produce anteiso and/or iso branched-chain fatty acids, the first condensation reaction in fatty acid synthesis is the reaction of acetyl-CoA with malonyl-ACP, yielding 3-ketobutyryl-ACP (Smirnova and Reynolds, J. Industrial Microbiology & Biotechnology 27: 246-51 (2001)). This reaction is primarily catalyzed by the fabH product, a 3-ketoacyl-ACP synthase. The E. coli 3-ketoacyl-ACP synthase, however, shows specificity in that it prefers acetyl-CoA over branched acyl-CoA such as 2-methylbutyryl-CoA (Choi et al., J. Bacteriology 182: 365-70 (2000)). Reducing or removing endogenous FabH activity through chemical inhibitors such as cerulenin or through genetic engineering (e.g., by creating a fabD and fabH double mutant) reduces the amount of straight chain fatty acids produced. If desired, branched-chain fatty acid production also is increased by removing or reducing a host cell's (e.g., E. coli's) capacity to make straight-chain fatty acids by, for example, incorporating fatty acid synthesis genes derived from Exiguobacterium to shorten the chain length and/or increase the amount of anteiso and/or iso branched-chain fatty acids.

Additionally or alternatively, gene knockouts or knockdowns that minimize the carbon flow to branch pathways not contributing to the anteiso or iso fatty acid formation are used. For example, in one aspect, isoleucine transaminase activity is attenuated to redirect carbon flow from isoleucine synthesis to anteiso branched-chain fatty acid synthesis (see FIGS. 2 and 17). In this regard, in an exemplary embodiment of the invention, the cell is genetically modified to reduce expression of ilvE or inhibit activity of the gene product. In one aspect of the invention, the cell is modified to generate a fadD mutant defective in converting a fatty acid to fatty acyl-CoA, the first step in fatty acid degradation. Fatty acid content is thereby increased. Alternatively or in addition, the cell is modified to attenuate branched-chain amino acid aminotransferase (BCAT) activity. Enzyme activity is attenuated (i.e., reduced or abolished) by, for example, mutating the coding sequence for the enzyme to create a non-function or reduced-function polypeptide, by removing the coding sequence for the enzyme from the cellular genome, by interfering with translation of an RNA transcript encoding the enzyme (e.g., using antisense oligonucleotides), or by manipulating the expression control sequences influencing expression of the enzyme.

Anteiso and/or iso branched-chain fatty acids are produced using any suitable cells or organisms, such as, for example, bacterial cells and other prokaryotic cells, yeast cells, or mammalian cells. In certain embodiments, the invention relates to cells, such as Escherichia cells (e.g., E. coli), which do not naturally produce anteiso and/or iso branched-chain fatty acids. These cells are engineered to produce anteiso and/or iso branched-chain fatty acids as described herein. In one aspect, the cells are modified to produce anteiso and/or iso branched-chain fatty acids at desired levels and with desired chain lengths. In addition, in certain embodiments, the engineered cells tolerate large amounts of anteiso and/or iso branched-chain fatty acids in the growth medium, plasma membrane, or lipid droplets, and/or produce anteiso and/or iso branched-chain fatty acids more economically than an unmodified cell by, e.g., using a less expensive feedstock, requiring less fermentation time, and the like.

In certain embodiments, cells or organisms that naturally produce anteiso and/or iso branched-chain fatty acids, such as Bacillus subtilis and Streptomyces avermitilis, are modified as described herein to produce higher levels of anteiso and/or iso branched-chain fatty acids compared to an unmodified cell or organism. Optimization is achieved, for example, by incorporating regulatory mutations that lead to higher levels of fatty acids in the cells and/or overexpressing enzyme activities for increased branched keto acid precursor and/or precursors for the fatty acid biosynthesis pathway. Optimization also may be achieved by attenuating enzyme activity that diverts carbon flow from branched-chain fatty acid production. Alternatively or in addition, the cells produce anteiso and/or iso branched-chain fatty acids with specified chain lengths. If desired, a thioesterase is selected with specificity for a particular chain length. For example, the thioesterases from Mallard uropygial gland and rat mammary gland preferentially generate medium-chain length fatty acids having C6-C14 aliphatic tails.

Exemplary bacteria that naturally produce branched-chain fatty acids and are suitable for use in the invention include, but are not limited to, Spirochaeta aurantia, Spirochaeta littoralis, Pseudomonas maltophilia, Pseudomonas putrefaciens, Xanthomonas campestris, Legionella anisa, Moraxella catarrhalis, Thermus aquaticus, Flavobacterium aquatile, Bacteroides asaccharolyticus, Bacteroides fragilis, Succinimonas amylolytica, Desulfovibrio africanus, Micrococcus agilis, Stomatococcus mucilaginosus, Planococcus citreus, Marinococcus albusb, Staphylococcus aureus, Peptostreptococcus anaerobius, Ruminococcus albus, Sarcina lutea, Bacillus anthracis, Sporolactobacillus inulinus, Clostridium thermocellum, Sporosarcina ureae, Desulfotomaculum nigrificans, Listeria monocytogenes, Brochothrix thermosphacta, Renibacterium salmoninarum, Kurthia zopfii, Corynebacterium aquaticum, Arthrobacter radiotolerans, Brevibacterium fermentans, Propionibacterium acidipropionici, Eubacterium lentum, Cytophaga aquatilis, Sphingobacteriuma multivorumb, Capnocytophaga gingivalis, Sporocytophaga myxococcoides, Flexibacter elegans, Myxococcus coralloides, Archangium gephyra, Stigmatella aurantiaca, Oerskovia turbata, and Saccharomonospora viridis. Exemplary microorganisms that produce branched-chain fatty acids also are disclosed in, e.g., Kaneda, Microbiol. Rev. 55(2): 288-302 (1991) (see Table 3).

The polynucleotide(s) encoding one or more enzyme activities for producing anteiso and/or iso fatty acids may be derived from any source. Depending on the embodiment of the invention, the polynucleotide is isolated from a natural source such as bacteria, algae, fungi, plants, or animals; produced via a semi-synthetic route (e.g., the nucleic acid sequence of a polynucleotide is codon optimized for expression in a particular host cell, such as E. coli); or synthesized de novo. In certain embodiments, it is advantageous to select an enzyme from a particular source based on, e.g., the substrate specificity of the enzyme, the type of branched-chain fatty acid produced by the source, or the level of enzyme activity in a given host cell. In one aspect of the invention, the enzyme and corresponding polynucleotide are naturally found in the host cell and overexpression of the polynucleotide is desired. In this regard, in some instances, additional copies of the polynucleotide are introduced in the host cell to increase the amount of enzyme available for fatty acid production. Overexpression of a native polynucleotide also is achieved by upregulating endogenous promoter activity, or operably linking the polynucleotide to a more robust promoter.

Exogenous enzymes and their corresponding polynucleotides also are suitable for use in the context of the invention, and the features of the biosynthesis pathway or end product can be tailored depending on the particular enzyme used. If desired, the polynucleotide(s) is isolated or derived from the branched-chain fatty acid-producing organisms described herein. For example, E. coli FabH, a 3-ketoacyl-ACP synthase, preferentially uses acetyl-CoA as a substrate rather than branched acyl-CoA, while FabH from B. subtilis more efficiently drives branched fatty acid synthesis. Thus, in one aspect, the cell of the invention is an E. coli cell comprising a polynucleotide encoding B. subtilis FabH.

An exemplary citramalate synthase produced by the cell is derived from M. jannaschii CimA. Exemplary AHASs include E. coli IlvIH, E. coli IlvIH (G14D), E. coli IlvGM, and B. subtilis IlvBH. An exemplary BCDH is B. subtilis Bkd, and an exemplary 3-ketoacyl-ACP synthase is B. subtilis FabH. An exemplary threonine deaminase is E. coli TdcB. Exemplary thioesterases include, but are not limited to, E. coli TesA, thioesterase from Mallard uropygial gland, and thioesterase from rat mammary gland. An exemplary isopropylmalate isomerase is E. coli LeuCD, and an exemplary isopropylmalate dehydrogenase is E. coli LeuB. In one aspect, the cell comprises a nucleic acid sequence having at least about 90 percent identity to the nucleic acid sequence set forth in SEQ ID NO: 32, 36, 42, 43, 46, 51, 57, 62, 68, or 83, or encodes a polypeptide comprising an amino acid sequence having at least about 90 percent identity to the amino acid sequence set forth in SEQ ID NO: 33, 39, 40, 41, 47, 48, 52, 53, 58, 65, 66, 67, 84, or 85. Exemplary enzymes that mediate production of anteiso and/or iso fatty acids also are disclosed in Table A.

TABLE A

| Activity | Gene Name | Organism | Accession |
|---|---|---|---|
| Branched-chain amino acid | ilvE | Salmonella enteric | NP_457845 |
| transaminase | ilvE | Yersinia pestis | YP_002348774 |
| | ilvE | Shigella flexneri | NP_709575 |
| | ilvE | Pectobacterium carotovorum | YP_003018265 |
| | ilvE | Ralstonia solanacearum | YP_003753411 |
| Branched-chain 2-keto | bkdAA | Anoxybacillus flavithermus | YP_002315323 |
| acid dehydrogenase, E1 | bfmBAA | Staphylococcus aureus | NP_374631 |
| component, alpha subunit | bkdA1 | Sphingobium japonicum | YP_003544745 |
| | bkdA1 | Brevibacillus brevis | YP_002771850 |
| | bkdA | Lactobacillus casei | YP_001987607 |
| Branched-chain 2-keto | bkdAB | Anoxybacillus flavithermus | YP_002315324 |
| acid dehydrogenase, E1 | bfmBAB | Staphylococcus aureus | NP_374630 |
| component, beta subunit | bkdA2 | Sphingobium japonicum | YP_003544746 |
| | bkdA2 | Brevibacillus brevis | YP_002771851 |
| | bkdB | Lactobacillus casei | YP_001987606 |
| Branched-chain 2-keto | bkdB | Anoxybacillus flavithermus | YP_002315325 |
| acid dehydrogenase, E2 | bfmBB | Staphylococcus aureus | NP_374629 |
| component | pdhC | Sphingobium japonicum | YP_003544747 |
| | bkdB | Brevibacillus brevis | YP_002771852 |
| | bkdC | Lactobacillus casei | YP_001987605 |
| Branched-chain 2-keto | lpdV | Anoxybacillus flavithermus | YP_002315322 |
| acid dehydrogenase, E3 | | Staphylococcus aureus | NP_374632 |
| component | pdhD | Sphingobium japonicum | YP_003545508 |
| | Lpd | Brevibacillus brevis | YP_002771849 |
| | bkdD | Lactobacillus casei | YP_001987608 |
| 3-ketoacyl-ACP synthase | fabH | Geobacillus kaustophilus | YP_146657.1 |
| III | | Bacillus megaterium | YP_003561163.1 |
| | fabH | Staphylococcus aureus | ZP_05601460.1 |
| | fabH1 | Streptomyces coelicolor | P72392 |
| | | Beutenbergia cavernae | YP_002881824 |
| citramalate synthase | cimA | Methanobrevibacter | YP_003424156 |
| | | ruminantium | |
| | cimA | Leptospira interrogans | ABK13754 |
| | | Ignicoccus hospitalis | ABU82163 |
| | | Cyanothece 51142 | YP_001801665 |
| | | Geobacter sulfurreducens | NP_952848 |
| 3-isopropylmalate | leuB | Cronobacter turicensis | YP_003209069.1 |
| dehydrogenase | leuB | Shigella boydii | YP_406624 |
| | leuB | Actinobacillus | YP_001651485 |
| | | pleuropneumoniae | |
| | leuB | Cronobacter turicensis | YP_003209069 |
| | leuB | Pantoea ananatis | YP_003519000 |
| isopropylmalate isomerase | leuC | Salmonella enteric | NP_459116 |
| large subunit | leuC | Serratia proteamaculans | YP_001476977 |
| | leuC | Photorhabdus asymbiotica | YP_003039932 |
| | leuC | Klebsiella pneumoniae | YP_002917788 |
| | leuC | Haemophilus influenzae | NP_439151 |
| isopropylmalate isomerase | leuD | Shigella dysenteriae | YP_401823 |
| small subunit | leuD | Buchnera aphidicola | YP_002477704 |
| | leuD | Actinobacillus | YP_001967934 |
| | | pleuropneumoniae | |
| | leuD | Haemophilus somnus | YP_718599 |
| | leuD | Xanthomonas campestris | YP_365316 |
| threonine deaminase | Cha1 | Saccharomyces cerevisiae | NP_001018030 |
| | ilvA | Vibrio fischeri | YP_002157347 |
| | ilvA | Shewanella violacea | YP_003558613 |
| | ilvA | Methylococcus capsulatus | YP_112886 |
| | ilvA | Dichelobacter nodosus | YP_001209243 |
| threonine dehydratase | tdcB | Pantoea ananatis | YP_003519179 |
| | tdcB | Klebsiella pneumoniae | YP_001335951 |
| | tdcB | Shigella boydii | YP_409322 |
| | tdcB | Acinetobacter baumannii | YP_001706275 |
| | tdcB | Psychrobacter arcticum | YP_264671 |
| acetolactate synthase | ilvI | Yersinia enterocolitica | YP_001005008 |
| (AHAS) III large subunit | ilvI | Salmonella enterica | YP_002113134 |
| | ilvI | Buchnera aphidicola | NP_240056 |
| | ilvI | Xenorhabdus bovienii | YP_003469370 |
| | ilvI | Klebsiella pneumoniae | YP_001333772 |
| acetolactate synthase | ilvH | Laribacter hongkongensis | YP_002794162 |
| (AHAS) III small subunit | ilvH | Burkholderia mallei | YP_103450 |
| | ilvH | Nitrosomonas europaea | NP_841373 |
| | ilvH | Campylobacter jejuni | YP_178690 |
| | ilvH | Desulfomicrobium baculatum | YP_003156951 |
| acetolactate synthase | ilvG | Yersinia pestis | YP_002348776 |
| (AHAS) II large subunit | ilvG | Ralstonia solanacearum | YP_003752653 |
| | ilvG | Bordetella bronchiseptica | NP_887973 |
| | ilvG | Aeromonas salmonicida | YP_001140066 |
| | ilvG | Stenotrophomonas | YP_001973605 |
| | | maltophilia | |
| acetolactate synthase | ilvM | Shigella dysenteriae | YP_405398 |
| (AHAS) II small subunit | ilvM | Dickeya dadantii | YP_002989351 |
| | ilvM | Xenorhabdus bovienii | YP_003470064 |
| | ilvM | Photorhabdus luminescens | NP_931846 |
| | ilvM | Xanthomonas oryzae | YP_199583 |
| ketol-acid | ilvC | Buchnera aphidicola | NP_240398 |
| reductoisomerase | ilvC | Pseudomonas aeruginosa | YP_002442658 |
| | ilvC | Francisella tularensis | YP_001122021 |
| | ilvC | Vibrio fischeri | YP_205911 |
| | ilvC | Actinobacillus | YP_001652891 |
| | | pleuropneumoniae | |
| dihydroxy-acid | ilvD | Citrobacter rodentium | YP_003367413 |
| dehydratase | ilvD | Buchnera aphidicola | YP_002468875 |
| | ilvD | Xanthomonas campestris | YP_001901776 |
| | ilvD | Methylococcus capsulatus | YP_114512 |
| | ilvD | Chromobacterium violaceum | NP_900947 |
| enoyl-ACP reductase | fabI | Geobacillus | YP_001124839 |
| | | thermodenitrificans | |
| | fabI | Anoxybacillus flavithermus | YP_002316451 |
| | fabI | Listeria monocytogenes | ZP_05298370 |
| | fabI | Staphylococcus epidermidis | ZP_04796766 |
| | fabI | Clostridium thermocellum | ABN54364 |

In certain embodiments, the recombinant cell produces an analog or variant of the polypeptide encoding an enzyme activity involved in fatty acid biosynthesis. Amino acid sequence variants of the polypeptide include substitution, insertion, or deletion variants, and variants may be substantially homologous or substantially identical to the unmodified polypeptides as set out above. In certain embodiments, the variants retain at least some of the biological activity, e.g., catalytic activity, of the polypeptide. Other variants include variants of the polypeptide that retain at least about 50%, preferably at least about 75%, more preferably at least about 90%, of the biological activity.

Substitutional variants typically exchange one amino acid for another at one or more sites within the protein. Substitutions of this kind can be conservative, that is, one amino acid is replaced with one of similar shape and charge. Conservative substitutions include, for example, the changes of: alanine to serine; arginine to lysine; asparagine to glutamine; aspartate to glutamate; cysteine to serine; glutamine to asparagine; glutamate to aspartate; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

In some instances, the recombinant cell comprises an analog or variant of the exogenous or overexpressed polynucleotide(s) described herein. Nucleic acid sequence variants include one or more substitutions, insertions, or deletions, and variants may be substantially homologous or substantially identical to the unmodified polynucleotide. Polynucleotide variants or analogs encode mutant enzymes having at least partial activity of the unmodified enzyme. Alternatively, polynucleotide variants or analogs encode the same amino acid sequence as the unmodified polynucleotide. Codon optimized sequences, for example, generally encode the same amino acid sequence as the parent/native sequence but contain codons that are preferentially expressed in a particular host organism.

A polypeptide or polynucleotide “derived from” an organism contains one or more modifications to the native amino acid sequence or nucleotide sequence and exhibits similar, if not better, activity compared to the native enzyme (e.g., at least 70%, at least 80%, at least 90%, at least 95%, at least 100%, or at least 110% the level of activity of the native enzyme). For example, enzyme activity is improved in some contexts by directed evolution of a parent/native sequence. Additionally or alternatively, an enzyme coding sequence is mutated to achieve feedback resistance. The citramalate synthase CimA3.7 derived from M. jannaschii and described in Example 14 is truncated and comprises substitutions compared to the native M. jannaschii citramalate synthase enzyme. The modifications confer feedback resistance to the CimA3.7 enzyme and improve its activity. Similarly, substitution of the glycine with aspartate at amino acid position 14 of the E. coli IlvIH AHAS sequence (designated IlvIH (G14D)) imparts resistance to 2-aceto-2-hydroxy-butyrate inhibition. Thus, in one or more embodiments of the invention, the polypeptide encoded by the exogenous polynucleotide is feedback resistant and/or is modified to alter the activity of the native enzyme.

Recombinant cells can be produced in any suitable manner to establish an expression vector within the cell. The expression vector can include the exogenous polynucleotide operably linked to expression elements, such as, for example, promoters, enhancers, ribosome binding sites, operators and activating sequences. Such expression elements may be regulatable, for example, inducible (via the addition of an inducer). Alternatively or in addition, the expression vector can include additional copies of a polynucleotide encoding a native gene product operably linked to expression elements. Representative examples of useful promoters include, but are not limited to: the LTR (long terminal 35 repeat from a retrovirus) or SV40 promoter, the E. coli lac, tet, or trp promoter, the phage Lambda P_{L} promoter, and other promoters known to control expression of genes in prokaryotic or eukaryotic cells or their viruses. In one aspect, the expression vector also includes appropriate sequences for amplifying expression. The expression vector can comprise elements to facilitate incorporation of polynucleotides into the cellular genome. Introduction of the expression vector or other polynucleotides into cells can be performed using any suitable method, such as, for example, transformation, electroporation, microinjection, microprojectile bombardment, calcium phosphate precipitation, modified calcium phosphate precipitation, cationic lipid treatment, photoporation, fusion methodologies, receptor mediated transfer, or polybrene precipitation. Alternatively, the expression vector or other polynucleotides can be introduced by infection with a viral vector, by conjugation, by transduction, or by other suitable methods.

Cells, such as bacterial cells or any other desired host cells, containing the polynucleotides encoding the exogenous or overexpressed proteins are cultured under conditions appropriate for growth of the cells and expression of the polynucleotide(s). Cells expressing the polypeptide(s) can be identified by any suitable methods, such as, for example, by PCR screening, screening by Southern blot analysis, or screening for the expression of the protein. In certain embodiments, cells that contain the polynucleotide can be selected by including a selectable marker in the DNA construct, with subsequent culturing of cells containing a selectable marker gene, under conditions appropriate for survival of only those cells that express the selectable marker gene. The introduced DNA construct can be further amplified by culturing genetically modified cells under appropriate conditions (e.g., culturing genetically modified cells containing an amplifiable marker gene in the presence of a concentration of a drug at which only cells containing multiple copies of the amplifiable marker gene can survive). Cells that contain and express polynucleotides encoding the exogenous or overexpressed proteins are referred to herein as genetically modified cells. Bacterial cells that contain and express polynucleotides encoding the exogenous or overexpressed protein can be referred to as genetically modified bacterial cells.

In certain embodiments, the genetically modified cells (such as genetically modified bacterial cells) have an optimal temperature for growth, such as, for example, a lower temperature than normally encountered for growth and/or fermentation. For example, in certain embodiments, incorporation of branched-chain fatty acids into the membrane increases membrane fluidity, a property normally associated with low growth temperatures. In addition, in certain embodiments, cells of the invention exhibit a decline in growth at higher temperatures as compared to normal growth and/or fermentation temperatures as typically found in cells of the type.

Any cell culture condition appropriate for growing a host cell and synthesizing anteiso and/or iso fatty acids is suitable for use in the inventive method. Addition of fatty acid synthesis intermediates, precursors, and/or co-factors for the enzymes associated with anteiso and/or iso branched-chain fatty acid synthesis to the culture is contemplated herein. In one embodiment, the method comprises exposing the host cell to thiamine, which enhances anteiso fatty acid synthesis. Isoleucine, leucine, and/or valine is added to the culture in some embodiments.

The inventive method optionally comprises extracting anteiso and/or iso fatty acid from the culture. Fatty acids can be extracted from the culture medium and measured in any suitable manner. Suitable extraction methods include, for example, methods as described in Bligh et al., “A rapid method for total lipid extraction and purification,” Can. J. Biochem. Physiol. 37:911-917 (1959). In certain embodiments, production of fatty acids in the culture supernatant or in the membrane fraction of recombinant cells can be measured. In this embodiment, cultures are prepared in the standard manner, although nutrients (e.g. 2-methylbutyrate, isoleucine) that may provide a boost in substrate supply can be added to the culture. Cells are harvested by centrifugation, acidified with hydrochloric or perchloric acid, and extracted with chloroform and methanol, with the fatty acids entering the organic layer. The fatty acids are converted to methyl esters, using methanol at 100° C. The methyl esters are separated by gas chromatography (GC) and compared with known standards of straight-chain, iso and anteiso fatty acids (purchased from Larodan or Sigma). Confirmation of chemical identity is carried out by combined GC/mass spec, with further mass spec analysis of fragmented material carried out if necessary.

In one embodiment, the cell utilizes the branched-chain anteiso and/or iso fatty acid as a precursor to make or more other products. Products biosynthesized (i.e., derived) from anteiso or iso fatty acid include, but are not limited to, phospholipids, triglycerides, alkanes, olefins, wax esters, fatty alcohols, and fatty aldehydes. Some host cells naturally generate one or more products derived from anteiso or iso fatty acid; other host cells are genetically engineered to convert branched-chain fatty acid to, e.g., an alkane, olefin, wax ester, fatty alcohol, and/or fatty aldehyde. Organisms and genetic modifications thereof to synthesize products derived from branched-chain fatty acids are further described in, e.g., International Patent Publication Nos. WO 2007/136762, WO 2008/151149, and WO 2010/062480, and U.S. Patent Application Publication US 2010/0298612. In one aspect, the inventive method comprises extracting a product derived from anteiso fatty acid (phospholipid, triglyceride, alkane, olefin, wax ester, fatty alcohol, and/or fatty aldehyde synthesized in the cell from anteiso fatty acid) from the culture. Any extraction method is appropriate, including the extraction methods described in International Patent Publication Nos. WO 2007/136762, WO 2008/151149, and WO 2010/062480, and U.S. Patent Application Publication Nos. US 2010/0251601, US 20100242345, US 20100105963, and US 2010/0298612.

In one embodiment, the invention provides a cell comprising an exogenous or overexpressed polynucleotide comprising a nucleic acid sequence encoding a threonine deaminase, an exogenous or overexpressed polynucleotide comprising a nucleic acid sequence encoding an acetohydroxy acid synthase, an exogenous or overexpressed polynucleotide comprising a nucleic acid sequence encoding a branched-chain α-keto acid dehydrogenase, and an exogenous or overexpressed polynucleotide comprising a nucleic acid sequence encoding a 3-ketoacyl-ACP synthase. The cell optionally further comprises at least one exogenous or overexpressed polynucleotide comprising a nucleic acid sequence encoding a thioesterase. The polynucleotides are expressed in the cell. In one aspect, the cell is a bacterial cell that does not naturally produce anteiso fatty acid, such as Escherichia coli. The invention further provides a method of producing anteiso fatty acid, the method comprising culturing the bacterial cell under conditions that allow expression of the polynucleotides and production of anteiso fatty acid.

In another embodiment, the invention provides a cell comprising an exogenous or overexpressed polynucleotide comprising a nucleic acid sequence encoding a citramalate synthase (such as M. jannaschii CimA or a feedback resistant derivative thereof), an exogenous or overexpressed polynucleotide comprising a nucleic acid sequence encoding a branched-chain α-keto acid dehydrogenase (such as B. subtilis Bkd), and an exogenous or overexpressed polynucleotide comprising a nucleic acid sequence encoding a 3-ketoacyl-ACP synthase (such as B. subtilis FabH), wherein the polynucleotides are expressed in the cell. The cell optionally further comprises at least one exogenous or overexpressed polynucleotide comprising a nucleic acid sequence encoding an isopropylmalate isomerase, an isopropylmalate dehydrogenase, an acetohydroxy acid synthase (such as E. coli IlvIH, E. coli IlvIH (G14D), E. coli IlvGM, or B. subtilis IlvBH), a thioesterase, or a combination thereof.

The inventive cell preferably produces more anteiso fatty acid than an otherwise similar cell that does not comprise the polynucleotide(s). Methods of measuring fatty acid released into the fermentation broth or culture media are described herein. Anteiso fatty acid production is not limited to fatty acid accumulated in the culture, however, but also includes fatty acid used as a precursor for downstream reactions yielding products derived from anteiso fatty acid. Thus, products derived from anteiso (or iso) fatty acid (e.g., phospholipids, triglycerides, fatty alcohols, wax esters, fatty aldehydes, and alkanes) are, in some embodiments, surrogates for measuring branched-chain fatty acid production in a host cell. Methods of measuring fatty acid content in phospholipid in the cell membrane are described herein. Similarly, measurement of degradation products of branched-chain anteiso fatty acids also is instructive as to the amount of anteiso fatty acid is produced in a host cell. Depending on the particular embodiment of the invention, the least 50% more anteiso fatty acid than an otherwise similar cell that does not comprise the polynucleotide(s).

The following examples further describe and demonstrate embodiments within the scope of the present invention. The examples are given solely for the purpose of illustration and are not to be construed as limitations of the present invention, as many variations thereof are possible without departing from the spirit and scope of the invention.

### Example 1

### Construction of Bacillus subtilis bkd Expression Vectors

This example demonstrates production of a recombinant expression vector for expression of B. subtilis bkd in, e.g., E. coli.

Genomic DNA was prepared from B. subtilis 168 (Bacillus Genetic Stock Center, Columbus, Ohio) by picking a colony from an agar plate, suspending the colony in 100 μl of 1 mM Tris pH 8.0, 0.1 mM EDTA, boiling the sample for five minutes, and removing the insoluble debris by centrifugation.

B. subtilis bkd cDNA (SEQ ID NO: 1) (including lpdV, bkdAA, bkdAB, and bkdB genes that are part of the larger bkd operon in B. subtilis), was amplified from the genomic DNA sample by polymerase chain reaction (PCR) using primers BKD1 (SEQ ID NO: 2) and BKD2 (SEQ ID NO: 3) 5′, which incorporated flanking restriction sites for ApaI and MluI into the bkd cDNA during the PCR reaction.

The PCR was performed with 10 μl of Pfu Ultra II Hotstart 2× master mix (Agilent Technologies, Santa Clara, Calif.), 1 μl of a mix of the two primers (10 μmoles of each), 1 μl of B. subtilis genomic DNA, and 8 μl of water. The PCR began with a two-minute incubation at 95° C., followed by 30 cycles of 20 seconds at 95° C. for denaturation, 20 seconds for annealing at an optimized temperature of 62° C., and 90 seconds at 72° C. for extension. The samples were incubated at 72° C. for three minutes and then held at 4° C. The PCR product was purified using a QIAquick® PCR Purification Kit (Qiagen, Valencia, Calif.) and digested with ApaI and MluI restriction enzymes.

Bacterial expression vector pZA31-MCS (Expressys, Ruelzheim, Germany) was digested with ApaI, MluI, and HindIII, and the digested vector and insert were ligated together using Fast-Link (Epicentre Biotechnologies, Madison, Wis.). The ligation mix was then used to transform E. coli TOP10 cells (Invitrogen, Carlsbad, Calif.). Recombinant plasmids were isolated using a QIAPrep® Spin Miniprep Kit (Qiagen) spin plasmid miniprep kit and characterized by gel electrophoresis of restriction digests with EcoRV and with PstI. Plasmid DNA was isolated, and DNA sequencing confirmed that the bkd insert had been cloned and that the insert encoded the published amino acid sequence (Genbank # AL009126.3) (SEQ ID NO: 4). The resulting plasmid was designated pZA31-Bs bkd.

### Example 2

### Construction of B. subtilis fabHA Expression Vectors

This example demonstrates production of recombinant expression vectors for expression of B. subtilis fabHA in, e.g., E. coli.

To engineer E. coli for more efficient incorporation of the 2-methylbutyryl-CoA as a primer in fatty acid synthesis, E. coli was transformed with a vector containing B. subtilis fabHA, which encodes a 3-ketoacyl-ACP synthase that efficiently acts on 2-methylbutyryl-CoA. B. subtilis encodes two fabH genes whose products catalyze this reaction. Each fabH gene was separately cloned.

Genomic DNA was prepared from B. subtilis 168 (Bacillus Genetic Stock Center, Columbus, Ohio) by picking an isolated colony from a Luria agar plate, suspending the colony in 50 μl, of sterile Milli-Q water (Millipore, Bedford, Mass.), boiling the sample at 100° C. for five minutes, and removing the insoluble debris by centrifugation.

To generate an expression plasmid lacking a polyhistidine tag, B. subtilis fabHA cDNA was amplified from the genomic DNA sample by PCR using primers Bs_{—}939_fabHA_nco_U38 (SEQ ID NO: 5) and Bs_{—}939_fabHA_pst_L30 (SEQ ID NO: 6), which incorporated flanking restriction sites for NcoI and PstI into the amplified cDNA. Because of the use of an NcoI site in this cloning construct, an additional three base pairs was added to fabHA so that one would predict an extra alanine to be found in the FabHA protein.

To generate an expression plasmid where fabHA is fused to a polyhistidine tag, B. subtilis fabHA cDNA (SEQ ID NO: 7) was amplified from the genomic DNA sample by PCR using primers Bs_{—}939_fabHA_xho_U38 (SEQ ID NO: 8) and Bs_{—}939_fabHA_pst_L30 (SEQ ID NO: 9), which incorporated flanking restriction sites for XhoI and PstI into the amplified cDNA.

PCR was run on samples having 1 μl of B. subtilis 168 genomic DNA, 1.5 μl of a 10 μM stock of each primer, 5 μl of 10×Pfx reaction mix (Invitrogen Carlsbad, Calif.), 0.5 μl of Pfx DNA polymerase (1.25 units), and 41 μl of water. PCR conditions were as follows: the samples were initially incubated at 95° C. for one minute, followed by 30 cycles at 95° C. for 30 seconds (strand separation), 58° C. for 30 seconds (primer annealing), and 68° C. primer extension for 1.5 minutes. Following these cycles, there was a ten-minute incubation at 68° C., and the samples were then held at 4° C.

The PCR products were purified using a QIAquick® PCR Purification Kit (Qiagen), double digested with restriction enzymes XhoI/PstI or NcoI/PstI, and ligated (Fast-Link Epicentre Biotechnologies, Madison, Wis.) into XhoI/PstI or NcoI/PstI-digested pBAD/His A (Invitrogen, Carlsbad, Calif.). The ligation mix was used to transform E. coli DH5α™ (Invitrogen Carlsbad, Calif.). Isolated colonies were screened by PCR using a sterile pipette-tip stab as an inoculum into a reaction tube containing only water, followed by addition of the remaining PCR reaction cocktail (AccuPrime™ SuperMixII, Invitrogen Carlsbad, Calif.) and primers as described above.

Recombinant plasmids were isolated and purified using the QIAPrep® Spin Miniprep Kit (Qiagen) and characterized by restriction enzyme digestion (XhoI+PstI, NcoI+PstI, DraI, MfeI, and HaeII from Invitrogen or New England Biolabs, Beverly, Mass.). The plasmids were subsequently used to transform E. coli strain BW25113 (E. coli Genetics Stock Center, New Haven, Conn.) made competent using the calcium chloride method. Transformants were selected on Luria agar plates containing 100 ng/ml ampicillin. Plasmid DNA was isolated and purified using the QIAfilter™ Plasmid Midi Kit (Qiagen). DNA sequencing confirmed that the fabHA inserts had been cloned and that the inserts encoded the published amino acid sequence (SEQ ID NO: 10). The resulting plasmid lacking a polyhistidine tag was designated Bs fabHA-His and the plasmid incorporating a polyhistidine tag was designated pBAD-Bs fabHA+His.

### Example 3

### Construction of B. subtilis fabHB Expression Vectors

This example demonstrates production of recombinant expression vectors for expression of B. subtilis fabHB in, e.g., E. coli.

Genomic DNA was prepared from B. subtilis 168 (Bacillus Genetic Stock Center, Columbus, Ohio) by picking an isolated colony from a Luria agar plate, suspending the colony in 50 μL of sterile Milli-Q water (Millipore, Bedford, Mass.), boiling the sample at 100° C. for five minutes, and removing the insoluble debris by centrifugation.

To generate an expression plasmid lacking a polyhistidine tag, B. subtilis fabHB cDNA was amplified from the genomic DNA sample by PCR using primers RC_Bs_{—}978_fabHB_nco_U36 (SEQ ID NO: 11) and RC_Bs_{—}978_fabHB_pst_L32 (SEQ ID NO: 12), which incorporated flanking restriction sites for NcoI and PstI into the amplified cDNA. Because of the use of an NcoI site in this cloning a predicted serine-to-alanine change was made in the FabHB protein.

To generate an expression plasmid where fabHB would be fused to a polyhistidine tag, B. subtilis fabHB cDNA (SEQ ID NO: 13) was amplified from the genomic DNA sample by PCR using primers RC_Bs_{—}978_fabHB_xho_U41 (SEQ ID NO: 14) and RC_Bs_{—}978_fabHB_pst_L35 (SEQ ID NO: 15), which incorporated flanking restriction sites for XhoI and PstI into the amplified cDNA.

PCR was run on samples having 1 μl of B. subtilis 168 genomic DNA, 1.5 μl of a 10 μM stock of each primer, 5 μl of 10×Pfx reaction mix (Invitrogen Carlsbad, Calif.), 0.5 μl of Pfx DNA polymerase (1.25 units), and 41 μl of water. PCR conditions were as follows: the samples were initially incubated at 95° C. for one minute, followed by 30 cycles at 95° C. for 30 seconds (strand separation), 58° C. for 30 seconds (primer annealing), and 68° C. primer extension for 1.5 minutes. Following these cycles, there was a ten-minute incubation at 68° C., and the samples were then held at 4° C.

The PCR products were purified using a QIAquick® PCR Purification Kit (Qiagen), double digested with restriction enzymes XhoI/PstI or NcoI/PstI, and ligated (Fast-Link Epicentre Biotechnologies, Madison, Wis.) into XhoI/PstI or NcoI/PstI-digested pBAD/His A (Invitrogen, Carlsbad, Calif.). The ligation mix was used to transform E. coli DH5α™ (Invitrogen Carlsbad, Calif.). Isolated colonies were screened by PCR using a sterile pipette-tip stab as an inoculum into a reaction tube containing only water, followed by addition of the remaining PCR reaction cocktail (AccuPrime™ SuperMixII, Invitrogen Carlsbad, Calif.) and primers as described above.

Recombinant plasmids were isolated and purified using the QIAPrep® Spin Miniprep Kit (Qiagen) and characterized by restriction enzyme digestion (XhoI+PstI, NcoI+PstI, DraI, MfeI, and HaeII from Invitrogen or New England Biolabs, Beverly, Mass.). The plasmids were subsequently used to transform E. coli strain BW25113 (E. coli Genetics Stock Center, New Haven, Conn.) made competent using the calcium chloride method. Transformants were selected on Luria agar plates containing 100 μg/ml ampicillin. Plasmid DNA was isolated and purified using the QIAfilter™ Plasmid Midi Kit (Qiagen). DNA sequencing confirmed that the fabHB inserts had been cloned and that the inserts encoded the FabHB amino acid sequence (SEQ ID NO: 16). The resulting plasmid lacking a polyhistidine tag was designated Bs fabHB-His and the plasmid incorporating a polyhistidine tag was designated pBAD-Bs fabHB+His.

### Example 4

### Co-Transformation of E. coli with B. subtilis fabHA, fabHB and bkd Genes

This example demonstrates the co-transformation of E. coli with B. subtilis fabHA, fabHB and bkd genes.

To produce anteiso fatty acids in E. coli, combinations of each of the B. subtilis fabH plasmid constructs (Examples 2 and 3), with and without the B. subtilis bkd plasmid construct (Example 1), were used to transform both parent BW25113 and knockout BW25113 ΔfadD730 strains (E. coli Genetic Stock Center, New Haven, Conn.). The ΔfadD730 strain has a deleted acyl-CoA synthase gene. Acyl-CoA synthase is an enzyme in the fatty acid degradation pathway, and deletion of the acyl-CoA synthase gene increases fatty acid content in the host cell by attenuating the cell's natural fatty acid degradation pathway.

The two E. coli strains were made chemically competent for plasmid DNA transformation by a calcium chloride method. Actively growing 50 ml E. coli cultures were grown to an optical density (at 600 nm) of ˜0.4. Cultures were quickly chilled on ice, and the bacteria were recovered by centrifugation at 2700×g for 10 minutes. The supernatant was discarded and pellets were gently suspended in 30 ml of an ice-cold 80 mM MgCl₂, 20 mM CaCl₂ solution. Cells were again recovered by centrifugation at 2700×g for 10 minutes. The supernatant was discarded and pellets were gently resuspended in 2 ml of an ice-cold 0.1 M CaCl₂ solution.

The competent cells were used directly for the following co-transformations:

pBAD-Bs fabHA and pZA31-Bs bkd
pBAD-Bs fabHA-His and pZA31-Bs bkd
pBAD-Bs fabHB and pZA31-Bs bkd
pBAD-Bs fabHB-His and pZA31-Bs bkd

Cells were transformed on ice in pre-chilled 14 ml round bottom centrifuge tubes. Approximately 25 ng of each plasmid was incubated on ice with 100 μl of competent cells for 30 minutes. The cells were heat shocked at 42° C. for 90 seconds and immediately placed on ice for 2 minutes. 500 μl of pre-warmed SOC medium (Invitrogen, Carlsbad, Calif.) was added and the cells allowed to recover at 37° C. with 225 rpm shaking. 50 μl of the transformed cell mix was spread onto selective LB agar 100 μg/ml ampicillin plates to select for cells carrying the pBAD-Bs fabH plasmids. 50 μl of the transformed cell mix was spread onto selective LB agar 34 mg/ml chloramphenicol plates to select for cells carrying the pZA31-Bs bkd plasmid. 150 μl of the transformed cell mix was spread onto selective LB agar 100 mg/ml ampicillin and 34 mg/ml chloramphenicol plates to select for cells carrying both the pBAD-Bs fabH and pZA31-Bs bkd plasmids.

Individual colonies were picked from each plate and streaked onto all three varieties of LB agar plates to confirm the antibiotic resistance phenotype. Each strain was streaked to a single colony density, and a single colony was selected to be amplified for plasmid DNA isolation with QIAprep Spin Miniprep Kits (Qiagen, Valencia, Calif.). Restriction endonuclease digestion analysis of isolated plasmid DNA with HaeII verified the plasmid DNA pool for each strain.

### Example 5

### Construction of an Expression Vector for the Expression of Medium Branched-Chain Fatty Acid Thioesterase from Mallard Uropygial Gland

This example demonstrates the construction of an expression vector for the expression of a medium branched-chain fatty acid thioesterase from Mallard uropygial gland.

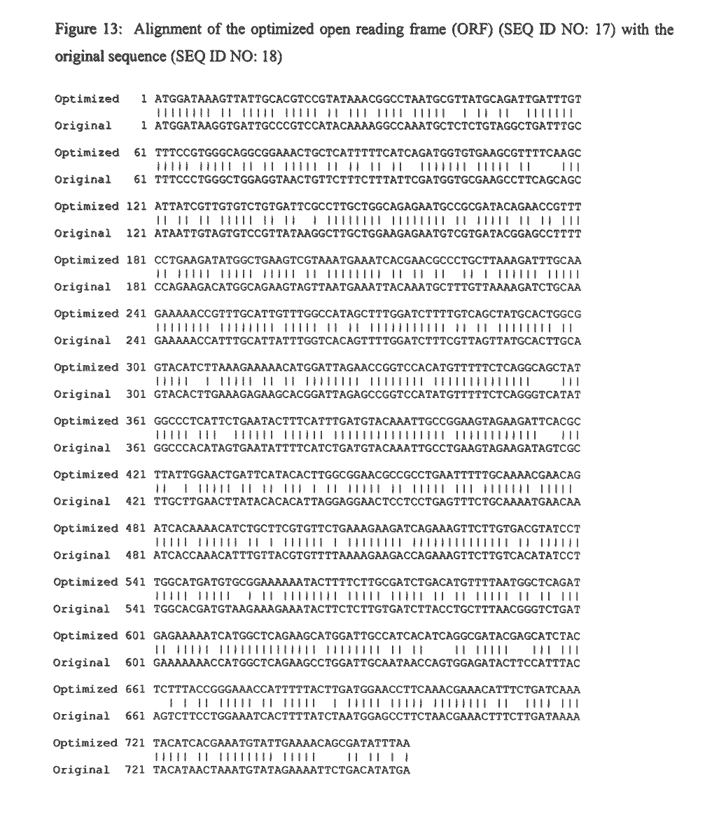

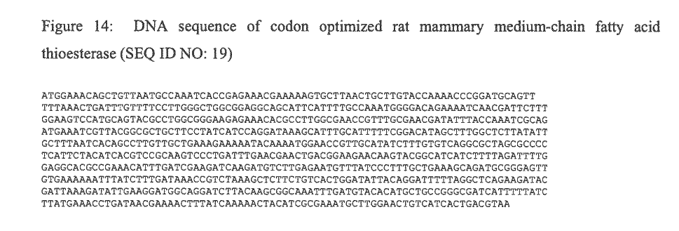

The coding sequence of the thioesterase (AAA49222.1) was codon optimized for B. subtilis expression. An alignment of the optimized open reading frame (ORF) (SEQ ID NO: 17) with the original sequence (SEQ ID NO: 18) is shown in FIG. 13. The optimized ORF was synthesized (GenScript) and inserted between the NcoI and BamHI sites of expression vector pTrcHisA (Invitrogen).

### Example 6

### Construction of an Expression Vector for the Expression of Medium Branched-Chain Fatty Acid Thioesterase from Rat Mammary Gland

This example demonstrates construction of the vector for the expression of a medium branched-chain fatty acid thioesterase from rat mammary gland.

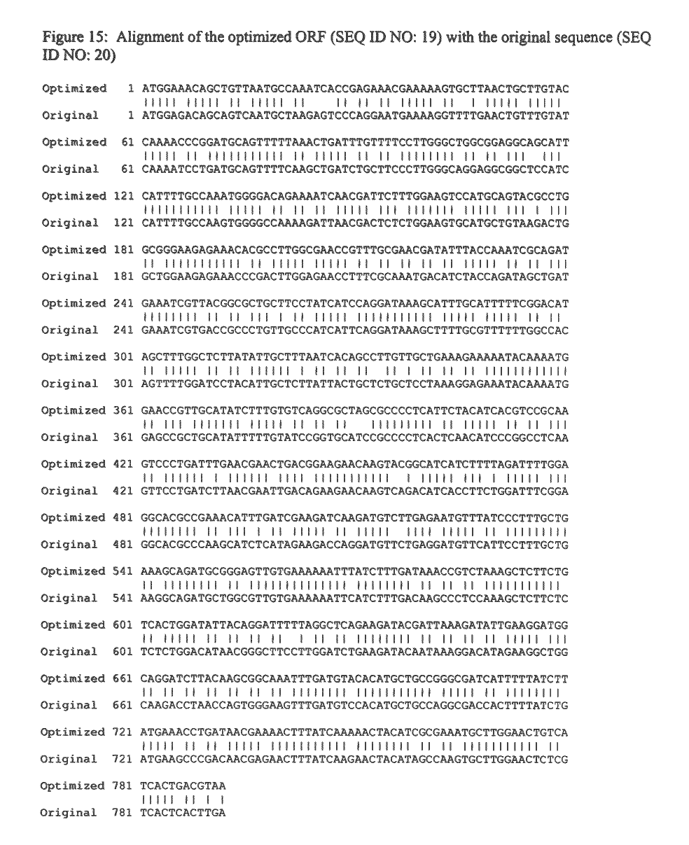

The coding sequence (AAA41578.1) was codon optimized for B. subtilis expression. An alignment of the optimized ORF (SEQ ID NO: 19) with the original sequence (SEQ ID NO: 20) is shown in FIG. 15. The optimized ORF was synthesized (GenScript) and inserted between the NcoI and BamHI sites of expression vector pTrcHisA (Invitrogen).

### Example 7

### Extraction of Lipids from Culture Medium

This example demonstrates the extraction of lipids from the culture medium. Lipids released from cells were extracted as follows: E. coli cells were grown in Luria Broth, Miller (BD, Sparks, Md.) to an optical density (600 nm) of at least 2 absorbance units. After centrifugation to pellet the cells, the supernatant was transferred to a fresh tube, and hydrochloric acid was added to a final pH between 1 and 2. Alternatively, to concentrate the supernatant, 25-50 ml can be lyophilized (VirTis, Gardiner, N.Y.) and suspended in 1 ml water. New or solvent-cleaned all-glass Pyrex tubes (Corning, Lowell, Mass.) were used for all subsequent steps. Three ml of 1:2 (v/v) chloroform:methanol was added to each 1 ml of supernatant sample. To the tube containing the supernatant, 1 ml of sterile Milli-Q (Millipore, Bedford, Mass.) water was added followed by 1 ml of chloroform. After briefly centrifuging the tubes (1000 rpm) for five minutes at room temperature, the top aqueous phase was removed, and the bottom organic phase was transferred to a clean, pre-weighed, and labeled 2 ml V-Vial® (15-415, diam.×H 17 mm×61 mm; Corning). Samples were dried under nitrogen or open air.

### Example 8

### Phospholipid Hydrolysis, Extraction of Fatty Acids from Cells, and Esterification of Fatty Acids

This example describes phospholipid hydrolysis, extraction of fatty acids from cells, and esterification of fatty acids.

Fatty acids were extracted from the cells as follows: E. coli cells were grown in Luria Broth, Miller to an optical density (600 nm) of at least 2 absorbance units. After centrifugation (3700 rpm for 10 minutes) to pellet the cells, the supernatant was discarded. Two ml of 0.1 M NaCl+50 mM Tris-HCl (pH between 7.5-8.0) was added to the pellet, the tube was vortexed, spun (3700 rpm for 10 minutes) to pellet the cells, and the supernatant was discarded. Sterile Milli-Q water (2 ml) was added to the tube containing the cell pellet, vortexed thoroughly, and the tube contents were transferred to a clean, pre-weighed and labeled Corning V-Vial® with solid-top cap capacity (2.0 ml, screw-cap size, 415, diam.×H: 17 mm×61 mm) Aluminum foil was placed over vials containing the 2 ml pellet, and the sample was frozen at −80° C. for 30 minutes and placed into the Virtis Freezemobile (VirTis, Gardiner, N.Y.) lyophilizer overnight (at 27° C.).

Dry weights of lyophilized samples were recorded, chloroform (0.75 ml) and 15% sulfuric acid (in methanol) were added, and tubes were placed in a 100° C. heating block (in a shielded fume hood). After four hours, the reaction mixture was transferred using a glass Pasteur pipette to a 13×100 mm Pyrex tube (Corning). Chloroform (1 ml) and 1 M sodium chloride (1 ml) were added to each tube and mixed by hand prior to a brief spin at 1000 rpm for 5 minutes. Using a glass Pasteur pipette, the top aqueous layer was discarded and a saturating amount of anhydrous sodium sulfate (˜50 mg) was added to the tube. The remaining volume (˜1 ml) was carefully removed using a Pasteur pipette and transferred to a pre-weighed glass GC tube. The tube can be dried under nitrogen or overnight in a fume hood. To the dried tube, 1 g (˜700 μL) of chloroform was added along with 0.1 g (˜70 μL) of a 0.1 g/1 methyl benzoate solution.

### Example 9

### Analysis of Fatty Acid Methyl Esters

This example demonstrates gas chromatography and mass spectrometry analysis of fatty acid methyl esters.

Fatty acid methyl esters were analyzed by gas chromatography, using hydrogen as a carrier gas at an initial flow rate of 1 cm³/sec. The injector temperature was set at 275° C. and the FID detector at 340° C. The oven temperature was kept at 70° C. for 1 minute following a 1 μL injection (50:1 split) with a temperature ramp of 10° C./min or 3° C./min to 325° C. The siloxane column used on the HP GC 6890 was a J&W Scientific DB-1 (part #122-1131), 60 m×0.25 mm ID×0.1 μm film thickness.

Fatty acid methyl esters were also analyzed by gas chromatography and mass spectrometry, using helium as a carrier gas at an initial flow rate of 0.9 ml/min (7.98 psi, 36 cm/sec). The injector temperature was set at 250° C. The oven temperature was kept at 70° C. for one minute following a 1 μL injection (20:1 split) with a temperature ramp of 10° C./min to 325° C. The column type used on the HP GC 6890 was a HP-5 Crosslinked 5% PhMe (Silicone; HP Part No. 19091)-433) with a 30 m×0.25 mm×0.25 μm film thickness.

### Example 10

### Production of Anteiso and Iso Fatty Acids by BW25113 Harboring pBAD-Bs fabHA-His and pZA31-Bs bkd

This example demonstrates the production of anteiso and iso fatty acids by BW25113 harboring pBAD-Bs fabHA-His and pZA31-Bs bkd.

Cells (50 ml) were cultured in Luria broth (BD, Sparks, Md.). With the culture at an optical density (600 nm) of 0.4-0.6, arabinose (0.2%) was added to induce fabHA expression. Lipid was harvested from the cell pellet (Example 8) and examined by gas chromatography (Example 7), revealing peaks that matched the mobility of C15 anteiso fatty acid standards. The identification of these peaks was confirmed by gas chromatography followed by mass spectrometry.

This example illustrates a method of producing anteiso and iso fatty acids in a microbe that does not naturally produce anteiso fatty acids (E. coli) by expressing in the microbe heterologous polynucleotides encoding a 3-ketoacyl-ACP synthase (fabHA from B. subtilis) and a branched-chain α-keto acid dehydrogenase (bkd from B. subtilis).

### Example 11

### Increasing Anteiso or Iso Fatty Acid Production by Increasing the Respective Precursors Isoleucine, Leucine or Valine

This example demonstrates a method of increasing anteiso or iso fatty acid production by increasing precursors isoleucine, leucine or valine.

BW25113 harboring pBAD-Bs fabHA-His and pZA31-Bs bkd was cultured and its fatty acid profile characterized as described in Example 10. To demonstrate the influence of available isoleucine, a parallel culture was prepared in the presence of one gram per liter isoleucine.

The samples were separated by gas chromatography. The peak areas were calculated using an algorithm in ChemStation® software Rev A.06.06 [509]. These peak sizes were added for all anteiso fatty acids and, separately, for all iso fatty acids. The presence of 1 gram per liter isoleucine was associated with an increase in anteiso fatty acids and a decrease in iso fatty acids, as shown in FIG. 16.

The results of this example show that increasing carbon flow to the isoleucine pathway of branched fatty acid synthesis increases the amount of anteiso branched-chain fatty acid produced in the host cell.

### Example 12

### Analysis of Fatty Acids

This example describes a method for analyzing fatty acids, such as fatty acids produced in bacterial cells, using gas chromatography.

Samples for analysis were prepared as follows. Bacterial cultures (approximately 1.5 ml) were frozen in 2.0 ml glass vials and stored at −15° C. until ready for processing. Samples were chilled on dry ice for 30 minutes and then lyophilized overnight (˜16 hours) until dry. A 10 μl aliquot of internal standard (glyceryl trinonadecanoate (Sigma catalog number T4632-1G)) was added to each vial, followed by addition of 400 μL of 0.5 N NaOH (in methanol). The vial was capped and vortexed for 10 seconds. Samples were then incubated at 65° C. for 30-50 minutes, removed from the incubator, and 500 μl of boron trifluoride reagent (Aldrich catalog number B1252) was added. The samples were vortexed for 10 seconds. Samples were then incubated at 65° C. for 10-15 minutes and cooled to room temperature (approximately 20 minutes). Hexane (350 μl) was added, and the samples were vortexed for 10 seconds. If the phases did not separate, 50-100 μl of saturated salt solution (5 g NaCl to 5 ml water) was added, and the sample again vortexed for 10 seconds. At least 100 μl of the top hexane layer was placed into a gas chromatography (GC) vial, which was capped and stored at 4° C. or −20° C. until analysis.

Gas chromatography was performed as described in Table B below. A bacterial acid methyl ester standard (Sigma catalog number 47080-U) and a fatty acid methyl ester standard (Sigma catalog number 47885-U) were used to identify peaks in samples. A sample check standard using glyceryl tripalmitate 1 (Sigma catalog number T5888-1G) was employed to confirm esterification of samples. A blank standard (internal standard only) was used to assess background noise.

TABLE B

| Gas Chromatograph | HP 5890 GC Series II | | | | |
|---|---|---|---|---|---|
| Detector | FID 360° C. 40 ml/min Hydrogen, 400 ml/min | | | | |
| | Air | | | | |
| Carrier Gas | Helium | | | | |
| Quantitative Program | GC Chemstation A.09.03. (Agilent) | | | | |
| Column | VF-5ms 15M × 0.150 mm × 0.15 μm Varian | | | | |
| | catalog number CP9035 | | | | |
| Injection Liner | Gooseneck (with glass wool packing) | | | | |
| Injector | HP 7673 | | | | |
| Injection Syringe | 10 μL | | | | |
| Injection Mode | Split 25:1 | | | | |
| Injection volume | 4 μL (Plunger Speed = fast; 5 sample pumps) | | | | |
| Pre Injection Solvent | 2 samples | | | | |
| Washes | | | | | |
| Post Injection Solvent | 3 for both acetone and hexane | | | | |
| Washes | | | | | |
| Injector Temperature | 325° C. | | | | |
| Total Program Time | 16 minutes | | | | |
| | Initial | Initial | Rate | Final | Final |
| | Temp. | Time | (° C./ | Temp | Time |
| | (° C.) | (min) | min) | (° C.) | (min) |
| Thermal Program | 90 | 0.75 | 20.0 | 325 | 1.0 |
| | | | 25.0 | 350 | 2.5 |

### Example 13

### Increasing Anteiso Fatty Acid Production by Increasing Carbon Flow Through the Threonine-Dependent Pathway

There are two primary pathways responsible for production of 2-oxobutanoate (also known as α-ketobutyrate), which is an intermediate in the synthesis of 2-methylbutyryl-CoA, the primer for anteiso fatty acid synthesis. One pathway generates 2-oxobutanoate from threonine (FIG. 2), while the second pathway uses citramalate as a precursor (FIG. 17). This example demonstrates that increasing carbon flow through a pathway utilizing threonine increases anteiso fatty acid production in host cells.

An E. coli strain was modified to increase production of threonine deaminase and, in some instances, acetohydroxy acid synthase (AHAS). Threonine deaminase and AHAS are the first two enzyme activities in the threonine-dependent pathway for anteiso fatty acid production. Threonine deaminase promotes the conversion of threonine to 2-oxobutanoate, which is converted to 2-aceto-2-hydroxy-butyrate via AHAS. An expression vector comprising an E. coli threonine deaminase coding sequence, tdcB, operably linked to a trc promoter was constructed. An expression vector comprising a gene fusion wherein an AHAS III coding sequence, ilvIH, is fused to the tdcB coding sequence also was prepared.

To isolate tdcB, genomic DNA was prepared from E. coli BW25113 (E. coli Genetic Stock Center, Yale University, New Haven, Conn.) by picking an isolated colony from a Luria agar plate, suspending the colony in 100 μl Tris (1 mM; pH 8.0), 0.1 mM EDTA, boiling the sample for five minutes, and removing the insoluble debris by centrifugation. tdcB was amplified from the genomic DNA sample by PCR using primers GTGCCATGGCTCATA TTACATACGATCTGCCGGTTGC (SEQ ID NO: 2) and GATCGAATTCATCCTTAGGCGTCAACGAAACCGGTGATTTG (SEQ ID NO: 3). PCR was performed on samples having 1 μl of E. coli BW25113 genomic DNA, 1 μl of a 10 μM stock of each primer, 25 μl of Pfu Ultra II Hotstart 2× master mix (Agilent Technologies, Santa Clara, Calif.), and 22 μl of water. PCR conditions were as follows: the samples were initially incubated at 95° C. for two minutes, followed by three cycles at 95° C. for 20 seconds (strand separation), 56° C. for 20 seconds (primer annealing), and 72° C. primer extension for 30 seconds. In addition, 27 cycles were run at 95° C. for 20 seconds, 60° C. for 20 seconds, and 72° C. primer extension for 30 seconds. There was then a three-minute incubation at 72° C., and the samples were held at 4° C.

The PCR products were purified using a QIAquick® PCR Purification Kit (Qiagen), double digested with restriction enzymes HindIII and NcoI, and ligated (Fast-Link Epicentre Biotechnologies, Madison, Wis.) with HindIII/NcoI-digested pTrcHisA vector (Invitrogen, Carlsbad, Calif.). The ligation mix was used to transform OneShot Top10™ E. coli cells (Invitrogen, Carlsbad, Calif.). Transformants were selected on Luria agar plates containing 100 ng/ml ampicillin. The recombinant plasmid was isolated using a Qiagen HiSpeed Plasmid Midi Kit and characterized by gel electrophoresis of restriction digests with HindIII and NcoI. DNA sequencing confirmed that the tdcB insert had been cloned and that the insert encoded the published amino acid sequence (Genbank number U00096.2) (SEQ ID NOs: 4 and 33). The resulting plasmid was designated pTrcHisA Ec tdcB.

A gene fusion was constructed wherein AHAS genes were placed behind Ec tdcB so that both TdcB and the recombinant AHAS would be produced from the same message. In some instances, AHAS is encoded by two subunits. For example, E. coli AHAS III is encoded by two genes, IlvI (SEQ ID NO: 34) and IlvH (SEQ ID NO: 35). To fuse the AHAS III genes, ilvIH (SEQ ID NO: 36), to tdcB, ilvIH was amplified from the E. coli BW25113 genomic DNA sample PCR using primer sequences set forth in SEQ ID NO: 37 and SEQ ID NO: 38, which incorporated flanking restriction sites for EcoRI onto ilvIH during the PCR reaction.

The PCR was performed with 25 μl of Pfu Ultra II Hotstart 2× master mix (Agilent Technologies, Santa Clara, Calif.), 1 μl of a mix of the two primers (10 mmoles of each), 1 μl of E. coli BW25113 genomic DNA, and 23 μl of water. The PCR began with a two-minute incubation at 95° C., followed by two cycles of 20 seconds at 95° C. for denaturation, 20 seconds for annealing at 55° C., and 90 seconds at 72° C. for extension. The product was further amplified by 28 cycles of 20 seconds at 95° C. for denaturation, 20 seconds for annealing at 62° C., and 90 seconds at 72° C. for extension. The samples were incubated at 72° C. for three minutes and then held at 4° C. The PCR product was purified using a QIAquick® PCR Purification Kit (Qiagen, Valencia, Calif.) and digested with EcoRI restriction enzyme.

The bacterial expression vector pTrcHisA Ec tdcB (prepared as described above) was digested with EcoRI, and the digested vector and insert were ligated using Fast-Link (Epicentre Biotechnologies, Madison, Wis.). The ligation mix was then used to transform E. coli TOP10 cells (Invitrogen, Carlsbad, Calif.). Recombinant plasmids were isolated using a QIAPrep® Spin Miniprep Kit (Qiagen) and characterized by gel electrophoresis of restriction digests with XmnI. DNA sequencing confirmed that the ilvIH insert had been cloned and that the insert encoded the published amino acid sequences (ilvI, Swiss-Prot # P00893.2; ilvH Swiss-Prot # P00894.3) (SEQ ID NO: 39 and SEQ ID NO: 40, respectively). The resulting plasmid was designated pTrc Ec tdcB Ec ilvIH.

Carbon flow to 2-oxobutanoate is increased by the use of an AHAS III that is feedback insensitive to valine. Valine insensitivity is conferred by, for example, substituting an aspartic acid for glycine at the fourteenth amino acid (G14D) of IlvH (SEQ ID NO: 41; Vyazmensky et al., Biochemistry, 35: 10339-46 (1996)). An expression vector for expressing an E. coli tdcB gene followed by an E. coli ilvIH G14D was prepared. The fourteenth codon of E. coli ilvH, GGC (encoding glycine) was mutated to GAC (encoding aspartic acid) by site-directed mutagenesis (“SDM”) (GenScript, Piscataway, N.J.) using the plasmid pTrc Ec tdcB Ec ilvIH as a template. The generated SDM variant region was sub-cloned back into the original pTrc-Ec tdcB Ec ilvIH template, using an AatI site in the large subunit E. coli ilvI gene and an XbaI site in the multiple cloning site (MCS). The SDM variant region DNA sequence is provided as SEQ ID NO: 42, and the corresponding original DNA sequence is provided as SEQ ID NO: 43. DNA sequencing confirmed the SDM product as ilvIH (G14D). The SDM ilvH G14D open reading frame (ORF) is presented as SEQ ID NO: 41. Restriction digest of plasmid DNA with AflIII confirmed the presence of a new AflIII site created by the SDM. The resulting plasmid was designated pTrc Ec tdcB Ec ilvIH G14D.

E. coli AHAS II is the product of two genes, ilvG (SEQ ID NO: 44) and ilvM (SEQ ID NO: 45). AHAS II is not functionally active in E. coli K-12 strains due to a mutation in ilvG. To generate active AHAS II, ilvG and ilvM were synthesized according to the genome sequences of BL21 (DE3) (GenBank accession No. CP001509.3 from base 3840800 to 3842706). A NotI site was added to the 5′ end of ilvG and an EcoRI site was added to the 3′ end of ilvM. The synthesized gene (SEQ ID NO: 46) was ligated to pTrcHisA Ec tdcB at the NotI and EcoRI sites following tdcB. Both tdcB and ilvGM are designed to be transcribed by the same promoter. DNA sequencing confirmed that the ilvGM insert had been cloned and that the insert encoded the published amino acid sequences (GenBank Accession No. CAQ34112 (ilvG) and GenBank CAQ34113 (ilvM); SEQ ID NO: 47 and SEQ ID NO: 48, respectively). The resulting plasmid was designated pTrc Ec tdcB Ec ilvGM.

Similar to other AHAS enzymes, B. subtilis AHAS comprises products from two genes, ilvB (SEQ ID NO: 49) and ilvH (SEQ ID NO: 50). The B. subtilis AHAS genes were synthesized (GenScript, Piscataway, N.J.) using sequences from strain 168. An internal EcoRI site was present in the natural gene but removed from the synthetic gene to facilitate subsequent sub-cloning. A NotI site was added to the 5′ end of the ilvB sequence and an EcoRI site was added to the 3′ end of the ilvH sequence. The synthesized genes (SEQ ID NO: 51) were ligated to pTrcHisA Ec tdcB at the NotI and EcoRI sites following tdcB. Both tdcB and ilvBH are designed to be transcribed by the same promoter. DNA sequencing confirmed that the ilvBH insert had been cloned and that the insert encoded the published amino acid sequences (GenBank Accession No. CAA99561 (ilvB) and Swiss-Prot No. P37252.2 (ilvH); SEQ ID NO: 52 and SEQ ID NO: 53, respectively). The resulting plasmid was designated pTrc Ec tdcB Bs ilvBH.

To allow for E. coli to be transformed with an increased number of expression vectors, a portion of the polyhistidine-tagged B. subtilis fabHA vector (Example 2; pBAD Bs fabHA+His), including the regulation control gene araC and araBAD promoter (SEQ ID NO: 54), was cloned into a vector containing B. subtilis bkd (including lpdV, bkdAA, bkdAB, and bkdB genes of the larger bkd operon) (pZA31 Bs bkd).

The ClonEZ PCR cloning Kit (GenScript, Piscataway, N.J.) was utilized as follows: the target region was first amplified from the linearized pBAD Bs fabHA+His plasmid template by PCR using a (i) 5′ primer (SEQ ID NO: 55) containing 15 base pairs of homology sequence downstream (and including) the MluI site of pZA31 Bs bkd, and (ii) a 3′ primer (SEQ ID NO: 56) containing 15 base pairs of homology sequence upstream (and including) the MluI site of pZA31-Bs bkd. PCR was performed with 25 μl of Pfu Ultra II Hotstart 2× master mix (Agilent Technologies, Santa Clara, Calif.), 1 μl of a mix of the two primers (10 mmoles of each), 1 μl of linearized pBAD Bs fabHA+His (20 ng) plasmid DNA, and 23 μl of water. The PCR began with a two minute incubation at 95° C., followed by 30 cycles of 20 seconds at 95° C. for denaturation, 20 seconds for annealing at 62° C., and 90 seconds at 72° C. for extension. The samples were incubated at 72° C. for three minutes and then held at 4° C. The PCR product was purified using a QIAquick® PCR Purification Kit (Qiagen, Valencia, Calif.).

The ClonEZ reaction was set up with 6 μl (105 ng) of pZA31 Bs bkd (restriction digested with MluI), 8 μl (211 ng) of PCR-amplified insert, 2 μl of 10× ClonEZ buffer, 2 μl of 10× ClonEZ enzyme mix, and 2 μl of distilled water. The reaction proceeded for 30 minutes at 22° C. Some (8 μl) of the reaction mix was used to transform E. coli TOP-10 competent cells (Invitrogen, Carlsbad, Calif.). Isolated colonies were screened by PCR. Recombinant plasmids were isolated using a QIAPrep® Spin Miniprep Kit (Qiagen) and characterized by gel electrophoresis of restriction digests with HaeII and with EcoRV. DNA sequencing confirmed that the Ec araC Bs fabHA insert had been cloned into pZA31 Bs bkd and that the insert sequence matched the template sequence. The resulting plasmid was designated pZA31 Bs bkd fabHA.

An E. coli strain deficient in fatty acid degradation (Voelker et al., J. Bacteriology, 176: 7320-7327 (1994)) and able to regulate transcription of recombinant genes was generated. An E. coli K-12 strain defective in fadD, thus lacking fatty acyl-CoA synthetase, was used as starting material. The strain K27 (F-, tyrT58(AS), fadD88, mel-1; CGSC Strain No. 5478) was obtained from the E. coli Genetic Stock Center (New Haven, Conn.). A genomic regulation cassette from strain DH5αZ1 [laci^{q}, PN25-tetR, Sp^{R}, deoR, supE44, Δ(lacZYA-argFV169), φ80 lacZΔM15 (Expressys, Ruelzheim, Germany)] was transducted into the host strain. The transducing phage P1ᵥᵢᵣ was charged with DH5αZ1 DNA as follows. A logarithmically growing culture (5 ml LB broth containing 0.2% glucose and 5 mM CaCl₂) of donor strain, DH5αZ1, was infected with 100 μl of a lysate stock of P1ᵥᵢᵣ phage. The culture was further incubated three hours for the infected cells to lyse. The debris was pelleted, and the supernatant was further cleared through a 0.45 μm syringe filter unit. The fresh lysate was titered by spotting 10 μL of serial 1:10 dilutions of lysate in TM buffer (10 mM MgSO₄/10 mM Tris.Cl, pH 7.4) onto a 100 mm LB (with 2.5 mM CaCl₂) plate overlayed with a cultured lawn of E. coli in LB top agar (with 2.5 mM CaCl₂). The process was repeated using the newly created phage stock until the phage titer surpassed 10⁹ pfu/mL.

The higher titer phage stock was used to transduce fragments of the DH5αZ1 genome into a recipient K27 strain as follows. An overnight culture (1.5 ml) of K27 was pelleted and resuspended in 750 μl of a P1 salts solution (10 mM CaCl₂/5 mM MgSO₄). An aliquot (100 μl) of the suspended cells was inoculated with varying amounts of DH5αZ1 donor P1ᵥᵢᵣ lysate (1, 10, and 100 μl) in sterile test tubes. The phage was allowed to adsorb to the cells for 30 minutes at 37° C. The absorption period was terminated by addition of 1 ml LB broth plus 200 μl of 1 M sodium citrate, and the cultures were further incubated for 1 hour at 37° C. with aeration. The cultures were pelleted, the cells suspended in 100 μl of LB broth (plus 0.2 M sodium citrate), and were spread onto LB agar plates with 50 μg/mL spectinomycin. Spectinomycin-resistant strains were isolated, and genomic DNA was screened by PCR for the presence of tetR, lacl^{q} and fadD88. One such transductant was designated K27-Z1 and used in further studies.

To transform K27-Z1 cells, competent cells were placed on ice in pre-chilled 14 ml round bottom centrifuge tubes. Approximately 30 ng of each plasmid was incubated with 50 μl of chemically competent K27-Z1 cells (Cohen et al., Proceedings National Academy Sciences U.S.A., 69: 2110-4 (1972)) for 30 minutes. The cells were heat shocked at 42° C. for 90 seconds and immediately placed on ice for two minutes. Pre-warmed SOC medium (250 μl) (Invitrogen, Carlsbad, Calif.) was added, and the cells were allowed to recover at 37° C. with 125 rpm shaking for one hour. Transformed cell mix (20 μl) was spread onto selective LB agar with 100 μg/ml ampicillin to select for cells carrying any of the pTrc-HisA-based plasmids. Transformed cell mix (50 μl) was spread onto LB agar with 34 μg/ml chloramphenicol to select for cells carrying the pZA31 Bs bkd Bs fabH plasmid. Transformed cell mix (150 μl) was spread onto LB agar with 100 μg/ml ampicillin and 34 μg/ml chloramphenicol to select for cells carrying both the pTrc-HisA-based and pZA31 Bs bkd Bs fabH plasmids. In some cases, the creation of triple transformants required two transformations: a double transformant was originally created, made competent, and transformed by a third plasmid.

Individual colonies were picked for each strain and streaked to a single colony density on appropriate antibiotic selection plates. A single colony was selected to be amplified for plasmid DNA isolation with QIAprep Spin Miniprep Kits (Qiagen, Valencia, Calif.). Restriction endonuclease digestion analysis with AflIII of isolated plasmid DNA verified the plasmid DNA pool for each strain.

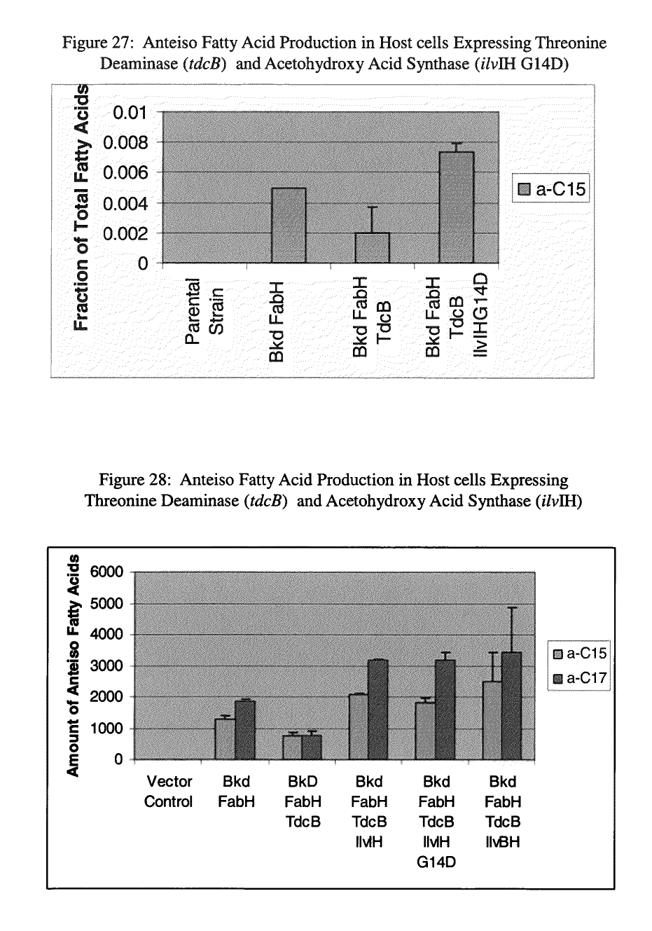

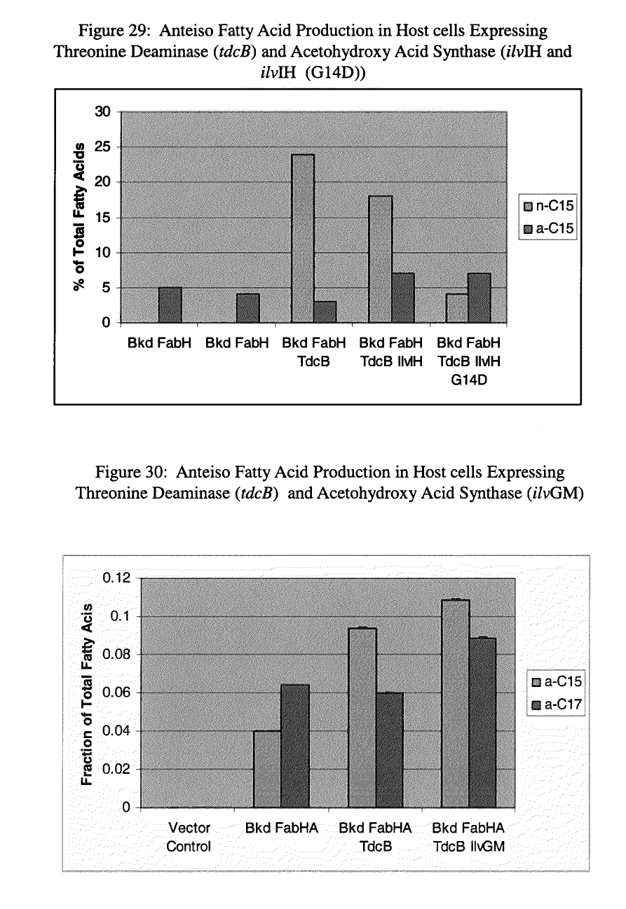

The resulting expression vectors were introduced into E. coli host cells comprising B. subtilis bkd and fabH, which were cultured in M9 glycerol medium comprising IPTG, tetracycline, and arabinose to induce recombinant gene expression. A sample of K27-Z1 comprising pZA31 Bs bkd fabHA and pTrc Ec tdcB Ec ilvGM was deposited with American Type Culture Collection (ATCC), 10801 University Blvd., Manassas, Va., on Dec. 14, 2010, under the provisions of the Budapest Treaty for the International Recognition of the Deposit of Microorganisms for the Purpose of Patent Procedure (“Budapest Treaty”), and assigned Deposit Accession No. [XXX] on [DATE]. Other AHAS genes also were tested for enhancement of anteiso fatty acid production. Fatty acids produced by the bacterial cells were isolated and separated. The amount of anteiso fatty acid produced by the modified bacteria was compared to the amount of fatty acid produced by an unmodified parental E. coli strain and E. coli producing B. subtilis Bkd and FabH. The quantity of each type of fatty acid was divided by the total amount of fatty acids produced. Unexpectedly, host cells expressing tdcB exhibited a decrease in anteiso fatty acid production. Co-expression of Ec tdcB (encoding threonine deaminase) and Ec ilvIH genes (encoding AHAS III) increased anteiso C15 fatty acid production in the E. coli strain also carrying Bs fabH and Bs bkd, relative to the E. coli strain carrying Bs fabH Bs bkd and that was not modified with the threonine-dependent pathway enzymes. Increased anteiso fatty acid production was observed for the valine-insensitive ilvIH G14D (FIG. 27), Ec ilvIH (FIGS. 28 and 29), the exogenous B. subtilis gene Bs ilvBH (FIG. 28), and E. coli ilvGM (FIG. 30).

The results of this example demonstrate that genetic modifications designed to increase carbon flow through the threonine-dependent pathway enhances anteiso fatty acid production.

### Example 14

### Increasing Anteiso Fatty Acid Production by Increasing Carbon Flow Through the Citramalate-Dependent Pathway

This example describes the generation of a recombinant microbe that produces exogenous citramalate synthase to further increase anteiso fatty acid production. The native Methanococcus jannaschii citramalate synthase coding sequence also was mutated through directed evolution to improve enzyme activity and feedback resistance to create cimA3.7 (SEQ ID NO: 58) (Atsumi et al., Applied and Environmental Microbiology 74: 7802-8 (2008)). E. coli is not known to have citramalate synthase activity, and a strain was engineered to produce exogenous citramalate synthase while overproducing several native E. coli enzymes: LeuB, LeuC, LeuD, and each of several AHASs. Citramalate synthase, LeuB, LeuC, LeuD, and IlvIH (G14D) mediate the first five chemical conversions in the citramalate pathway to produce anteiso fatty acids (FIG. 17).

To generate a synthetic CimA3.7 gene codon-optimized for E. coli expression, a DNA fragment (SEQ ID NO: 57) containing a restriction site BspHI (bases 1-6), codon-optimized cimA3.7 fragment (bases 3-1118), stop codon TGA (bases 1119-1121), a fragment of 52 bases from the start of the E. coli leuB gene (bases 1121-1173), and a linker sequence (bases 1174-1209) containing NotI, PacI, PmeI, XbaI and EcoRI sites was synthesized (GenScript, Piscataway, N.J.). The stop codon of cimA3.7 (TGA) and start codon (ATG) of leuB overlaps one base (A), presumably to enable translational coupling. This overlap mimics the native leuA and leuB coupling in E. coli. The synthesized fragment was digested with BspHI and EcoRI and cloned into pTricHisA (Invitrogen) at the NcoI and EcoRI sites, using the compatible ends generated by BspHI and NcoI. The end of the leuB fragment (bases 1168-1173) also contains a BspEI site (underlined) for cloning of leuBCD. This vector was designated as pTrcHisA Mj cimA.

The leuB (SEQ ID NO: 59) gene encodes 3-isopropylmalate dehydrogenase. The leuC (SEQ ID NO: 60) and leuD (SEQ ID NO: 61) genes encode isopropylmalate isomerase large subunit and small subunit, respectively. To fuse the three-gene complex leuBCD (SEQ ID NO: 57) behind Mj cimA, E. coli leuBCD cDNA was amplified from an E. coli BW25113 genomic DNA sample using PCR primers (SEQ ID NO: 63 and SEQ ID NO: 64), which included a BspEI restriction site in leuB and incorporated a NotI restriction site 3′ of the stop codon of leuD during the PCR reaction. The PCR was performed with 50 of Pfu Ultra II Hotstart 2× master mix (Agilent Technologies, Santa Clara, Calif.), 1 μl of a mix of the two primers (10 mmoles of each), 1 of E. coli BW25113 genomic DNA, and 48 μl of water. The PCR began with a two minute incubation at 95° C., followed by 30 cycles of 20 seconds at 95° C. for denaturation, 20 seconds for annealing at 64° C., and two minutes at 72° C. for extension. The sample was incubated at 72° C. for three minutes and then held at 4° C. The PCR product (leuBCD insert) was purified using a QIAquick® PCR Purification Kit (Qiagen, Valencia, Calif.).

The leuBCD insert and the bacterial expression vector pTrcHisA Mj cimA were digested with BspEI. The digested vector and leuBCD insert were again purified using a QIAquick® PCR purification columns prior to being restriction digested with NotI. Following final column purification, the digested vector and insert were ligated using Fast-Link (Epicentre Biotechnologies, Madison, Wis.). The ligation mix was then used to transform E. coli TOP10 cells (Invitrogen, Carlsbad, Calif.). Recombinant plasmids were isolated using a QIAPrep® Spin Miniprep Kit (Qiagen) and characterized by gel electrophoresis of restriction digests with AflIII. DNA sequencing confirmed that the leuBCD insert had been cloned and that the insert encoded the published amino acid sequences (GenBank Accession No. AAC73184 (Ec leuB) (SEQ ID NO: 65); GenBank Accession No. AAC73183 (Ec leuC) (SEQ ID NO: 66); and GenBank Accession No. AAC73182 (Ec leuD) (SEQ ID NO: 67)). The resulting plasmid was designated pTrc Mj cimA Ec leuBCD.

The AHAS genes (ilvIH, ilvIH G14D, ilvGM, and Bs ilvBH), flanked by 5′ NotI and 3′ EcoRI sites (described above), were cloned into the NotI and EcoRI sites of the expression plasmid pTrc Mj cimA Ec leuBCD and designated as follows:

E. coli AHAS III ilvIH (SEQ ID NO: 36)→pTrc Mj cimA Ec leuBCD Ec ilvIH

E. coli AHAS III ilvIH (G14D) (SEQ ID NO: 41)→pTrc Mj cimA Ec leuBCD Ec ilvIH (G14D)

E. coli BL21(DE3) AHAS II ilvGM (SEQ ID NO: 46)→pTrc Mj cimA Ec leuBCD Ec ilvGM

B. subtilis AHAS ilvBH (SEQ ID NO: 51)→pTrc Mj cimA Ec leuBCD Ec ilvBH.

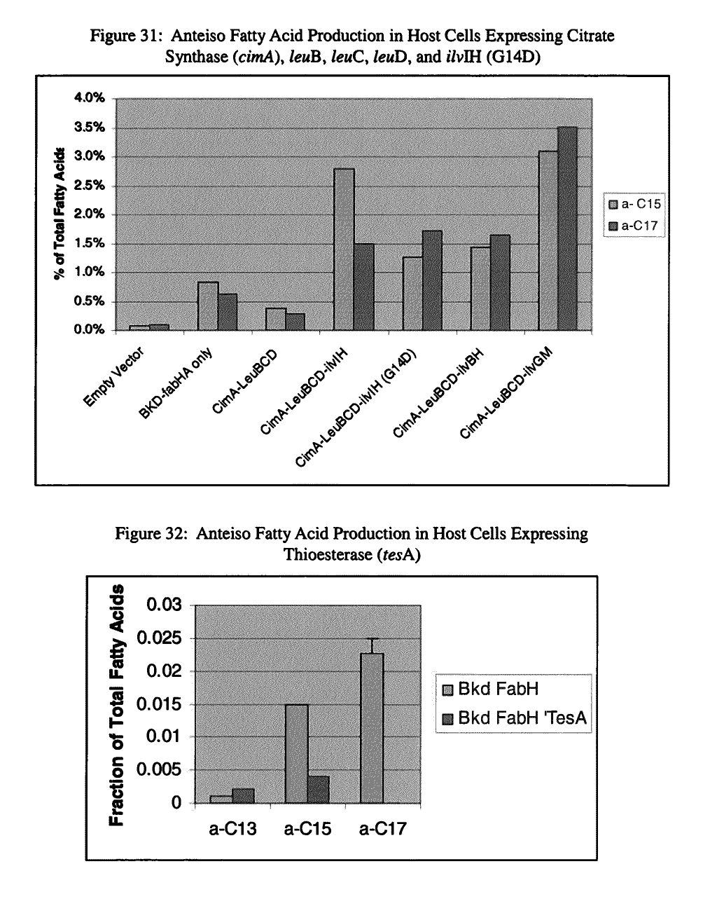

A sample of K27-Z1 comprising pZA31 Bs bkd fabHA and pTrc Mj cimA Ec leuBCD Ec ilvGM was deposited with American Type Culture Collection (ATCC), 10801 University Blvd., Manassas, Va., on Dec. 14, 2010, under the provisions of the Budapest Treaty for the International Recognition of the Deposit of Microorganisms for the Purpose of Patent Procedure (“Budapest Treaty”), and assigned Deposit Accession No. [XXX] on [DATE]. Expression of these recombinant polynucleotides in an E. coli host producing FabH and Bkd from B. subtilis further increased anteiso fatty acid production, as demonstrated for anteiso fatty acids of chain lengths of fifteen and seventeen carbons (FIG. 31).

### Example 15

### Tailoring Anteiso Fatty Acid Chain Length with Thioesterase

This example illustrates a method of tailoring anteiso fatty acid chain length using thioesterase. The method described herein is useful for, e.g., producing a pool of fatty acids of predetermined chain length for commercial applications.

An expression vector (pTrc Ec ‘tesA) was constructed comprising a nucleic acid sequence encoding the E. coli enzyme ‘TesA, which has thioesterase activity (Cho et al., J. Biological Chemistry, 270: 4216-9 (1995)). A truncated E. coli tesA (‘tesA) cDNA (SEQ ID NO: 68) was created by PCR amplification of the E. coli tesA gene (GenBank Accession No. L06182). A 5′ primer (SEQ ID NO: 69) was designed to anneal after the 26th codon of tesA, modifying the 27th codon from an alanine to a methionine and creating a NcoI restriction site. A 3′ primer (SEQ ID NO: 71) was designed to incorporate a BamHI restriction site. PCR was performed with 50 μl of Pfu Ultra II Hotstart 2× master mix (Agilent Technologies, Santa Clara, Calif.), 1 μl of a mix of the two primers (10 μmoles of each), 1 μl of E. coli BW25113 genomic DNA, and 48 μl of water. The PCR began with a two minute incubation at 95° C., followed by 30 cycles of 20 seconds at 95° C. for denaturation, 20 seconds for annealing at 58° C., and 15 seconds at 72° C. for extension. The sample was incubated at 72° C. for three minutes and then held at 4° C. The PCR product (Ec ‘tesA) was purified using a QIAquick® PCR Purification Kit (Qiagen, Valencia, Calif.). The bacterial expression vector pTrcHisA and ‘tesA PCR product were digested with NcoI and BamHI. The digested vector and insert were ligated using Fast-Link (Epicentre Biotechnologies, Madison, Wis.). The ligation mix was then used to transform E. coli TOP10 cells (Invitrogen, Carlsbad, Calif.). Recombinant plasmids were isolated using a QIAPrep® Spin Miniprep Kit (Qiagen) and characterized by gel electrophoresis of restriction digests with HaeII. DNA sequencing confirmed that the ‘tesA insert had been cloned and that the insert encoded the expected amino acid sequences (SEQ ID NO: 73). The resulting plasmid was designated pTrc Ec ‘tesA.

To limit gene expression, the truncated E. coli ‘tesA gene was subcloned into a low-copy bacterial expression vector pZS21-MCS (Expressys, Ruelzheim, Germany). The expression vector pTrc Ec ‘tesA was a template in a PCR reaction using a 5′ primer (SEQ ID NO: 74) designed to create a flanking XhoI restriction site and include the pTrcHisA lac promoter (to replace the pZS21-MCS vector tet promoter) and a 3′ primer (SEQ ID NO: 75) incorporating a HindIII restriction site. The PCR was performed with 50 μl of Pfu Ultra II Hotstart 2× master mix (Agilent Technologies, Santa Clara, Calif.), 1 μl of a mix of the two primers (10 μmoles of each), 1 μl of pTrc Ec ‘tesA plasmid DNA (6 ng), and 48 μl of water. The PCR began with a two-minute incubation at 95° C., followed by 30 cycles of 20 seconds at 95° C. for denaturation, 20 seconds for annealing at 57° C., and 20 seconds at 72° C. for extension. The sample was incubated at 72° C. for three minutes and then held at 4° C. The PCR product was purified using a QIAquick® PCR Purification Kit (Qiagen, Valencia, Calif.). The bacterial expression vector pZS21-MCS and the Ec ‘tesA PCR product were digested with XhoI and HindIII. The digested vector and insert were ligated using Fast-Link (Epicentre Biotechnologies, Madison, Wis.). The ligation mix was then used to transform E. coli TOP10 cells (Invitrogen, Carlsbad, Calif.). Recombinant plasmids were isolated using a QIAPrep® Spin Miniprep Kit (Qiagen) and characterized by gel electrophoresis of restriction digests with HaeII. DNA sequencing confirmed that the ‘tesA insert had been cloned and that the insert encoded the expected amino acid sequences (SEQ ID NO: 73). The resulting plasmid was designated pZS22 Ec ‘tesA.

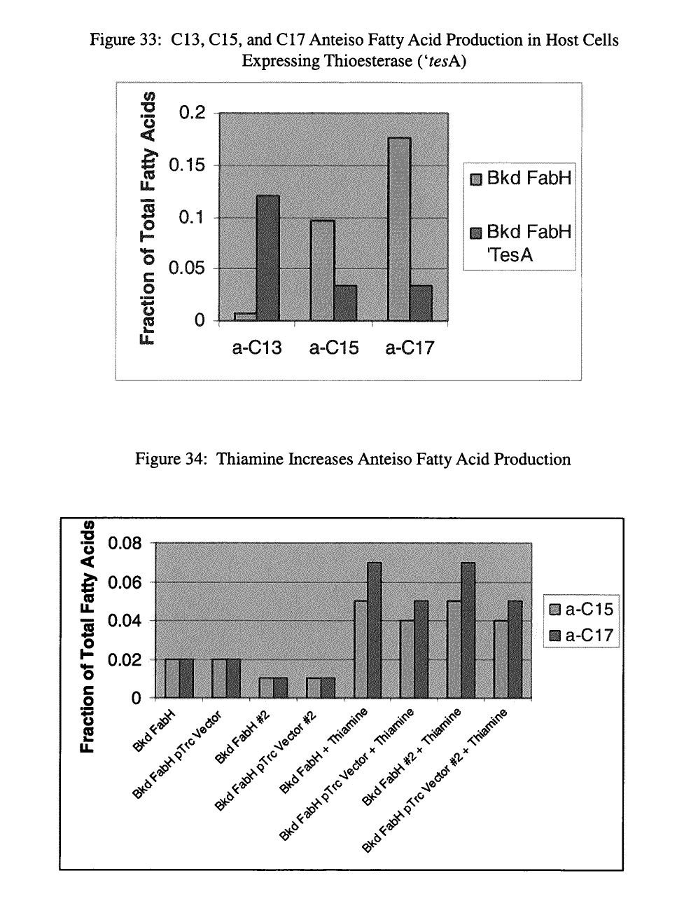

The expression vectors were introduced into E. coli host cells. Host cells producing ‘TesA generated more mid-chain-length (thirteen carbons) anteiso fatty acids and less longer-chain fatty acids (fifteen and seventeen carbons) compared to host cells that did not produce ‘TesA (FIG. 32). ‘tesA expression also led to the production of shortened anteiso fatty acids in a BL21 Star (DE3) strain of E. coli (FIG. 33). Surprisingly, the Ec ‘tesA-containing E. coli BL21 Star (DE3) strain produced more anteiso fatty acids than with the Ec ‘tesA-containing E. coli K-12 derivative strain.

This example demonstrates that overexpression of a thioesterase increases the proportion of medium chain length anteiso fatty acids (e.g., anteiso fatty acids 13 carbons in length) produced by a host microorganism.

### Example 16

### Thiamine Increases Anteiso Fatty Acid Synthesis

Thiamine (vitamin B1) is a cofactor for two enzymes (AHAS and Bkd) responsible for production of anteiso fatty acids. Thiamine was added to LB (modified for lower salt) and an increase in anteiso C15 and C17 fatty acids was observed (FIG. 34).

### Example 17

### Synthesis of Anteiso Fatty Acid in E. coli Producing Listeria FabH

This example demonstrates the production of anteiso and iso fatty acids by a microbe engineered to produce exogenous 3-ketoacyl-ACP synthase.

The L. monocytogenes 10403S 3-ketoacyl-ACP synthase III (fabH) gene (GenBank Accession No. FJ749129.1; SEQ ID NO: 77) was codon-optimized for expression in E. coli and synthesized to include 5′-XhoI and 3′-PstI restriction sites (SEQ ID NO: 78). The resulting synthesized and sequenced DNA was sub-cloned into a pMA vector (GENEART Inc., Toronto, ON, Canada). To generate an expression plasmid where Listeria fabHis fused to a polyhistidine tag, the pMA vector containing the L. monocytogenes fabH gene was digested with XhoI and PstI and ligated (Fast-Link Epicentre Biotechnologies, Madison, Wis.) with XhoI/PstI-digested pBAD/HisA (Invitrogen, Carlsbad, Calif.). The ligation mix was used to transform E. coli DH5α™ (Invitrogen Carlsbad, Calif.). Isolated colonies were screened by PCR using a sterile toothpick stab as an inoculum into a reaction tube containing only water, followed by addition of PCR reaction cocktail (AccuPrime™ SuperMixII, Invitrogen Carlsbad, Calif.) and primers as described above (SEQ ID NO: 79, SEQ ID NO: 80). Recombinant plasmids were isolated and purified using the QIAPrep® Spin Miniprep Kit (Qiagen) and characterized by restriction enzyme digestion (DraI, MfeI, and HaeII (New England Biolabs, Beverly, Mass.)). The plasmids were subsequently used to transform BW25113 (E. coli Genetics Stock Center, New Haven, Conn.) made competent using the calcium chloride method. Transformants were selected on Luria agar plates containing 100 μg/ml ampicillin. Plasmid DNA was isolated and purified using the QJAfilter™ Plasmid Midi Kit (Qiagen). The resulting plasmid incorporating a polyhistidine tag was designated pBAD Lm_fabH+. This plasmid and pZA31 were used together to transform BW25113.

Transduced cells were cultured in Luria broth. When the culture reached an optical density (600 nm) of 0.4-0.6, arabinose (0.2%) was added to induce fabH expression. Lipid was harvested from the cell pellet and examined by gas chromatography, revealing peaks that matched the mobility of C15 anteiso and C15 iso fatty acid standards. The identity of the peaks was confirmed by gas chromatography followed by mass spectrometry (FIG. 36).

This example demonstrates the production of anteiso and iso fatty acids by a microbe (E. coli strain BW25113) expressing exogenous 3-ketoacyl-ACP synthase (Listeria fabH) and exogenous branched-chain α-ketoacid dehydrogenase (Bacillus bkd).

### Example 18

### Acetohydroxy Acid Isomeroreductase and Dihydroxyacid Dehydratase Enhance Anteiso Fatty Acid Production

E. coli strains expressing recombinant Ec tdcB exhibit increased linear C15 (n-C15) fatty acid production (FIG. 29), suggesting that 2-oxobutanoate (also referred to as 2-ketobutyrate) gives rise to an increase in propionyl-CoA, which is used as a primer for synthesis of straight fatty acids with an odd number of carbons (FIG. 2). Production of a recombinant AHAS decreases n-C15 fatty acid levels and increases C15 anteiso (a-C15) fatty acid levels, suggesting depletion of 2-oxobutanoate by AHAS (FIG. 30). The greater effect is on n-C15 depletion, suggesting that not all 2-oxobutanoate is directed to anteiso fatty acid production. In one embodiment of the invention, IlvC and/or IlvD, the enzymes that catalyze the two chemical conversions following production of 2-oxobutanoate in the anteiso fatty acid synthesis pathway, are overexpressed to increase anteiso fatty acid production. To generate a transcriptional fusion of E. coli genes ilvC (encoded by the nucleic acid sequence set forth in GenBank Accession No. U00096.2 at position 3955993 to position 3957468; SEQ ID NO: 81) and ilvD (encoded by the nucleic acid sequence set forth in GenBank Accession No. U00096.2 at position 3951501 to position 3953351; SEQ ID NO: 82) encoding acetohydroxy acid isomeroreductase and dihydroxyacid dehydratase, respectively, codon-optimized synthetic DNA is flanked with XhoI and MluI sites (SEQ ID NO: 83) and ligated into the expression vector pZS22-MCS. The ilvCD genes are operably linked to a trc promoter from pTrc HisA. The insert encodes the trc promoter, lac operator, rrnB anti-termination sequences, T7 gene 10 translational enhancer, ribosome binding site, the published amino acid sequences for IlvC (GenBank Accession No. AAC76779) and IlvD (GenBank Accession No. AAT48208.1) (SEQ ID NO: 84 and SEQ ID NO: 85 respectively), and unique restriction sites for NotI, PmeI, EcoRI, and XbaI.

### Example 19

### Attenuation of Transaminase Activity Encoded by Ec ilvE in Anteiso Fatty Acid Biosynthesis

Carbon flow through the metabolic pathway for anteiso fatty acid production can be diverted to isoleucine production via the transaminase IlvE (FIG. 2). This example illustrates a method of enhancing anteiso fatty acid by attenuating IlvE activity.

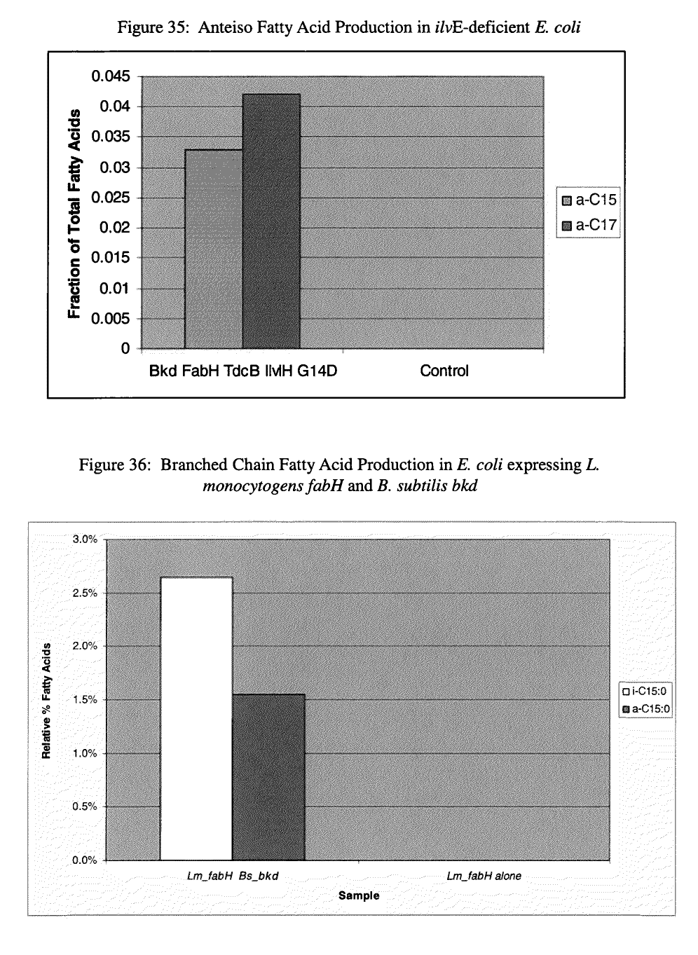

An ilvE deletion mutant, E. coli JW5606-1 (E. coli Genetic Stock Center, Yale University, New Haven, Conn.), was made chemically competent using calcium chloride. Cells were transformed with recombinant plasmids containing B. subtilis bkd, B. subtilis fabHA, E. coli tdcB, and E. coli ilvIH, or empty vector controls, pZA31MCS & pTrcHisA. Transformed cells (40 ml) were cultured in M9 minimal media supplemented with L-isoleucine, L-valine and L-leucine, each at 0.1% final concentration. When the culture reached an optical density (600 nm) of 0.4-0.6, arabinose (0.2%), isopropyl β-D-thiogalactopyranoside (1 mM) and anhydrotetracycline (100 ng/ml) were added to induce gene expression. After approximately 48 hours, lipid was harvested from the cell culture suspension, hydrolyzed, converted to methyl esters, and examined by gas chromatography. The presence of recombinant Bs fabHA, Bs bkd, Ec tdcB, and Ec ilvIH G14D led to anteiso fatty acid production in a strain deficient in Ec ilvE (FIG. 35).

### Example 20

### Construction of Enoyl-ACP Reductase Expression Vector

Enoyl-ACP reductase, the E. coli fabI product, catalyzes a rate-limiting step in fatty acid synthesis (Zheng et al., J. Microbiol. Biotechnol. 20: 875-80 (2010)). This example provides a method for producing an expression vector encoding an enoyl-ACP reductase. In some embodiments, an expression construct encoding enoyl-ACP reductase is introduced into a microbe that does not naturally generate branched fatty acids, such as E. coli, to enhance branched fatty acid production. In one embodiment, the enoyl-ACP reductase is modified to increase activity on branched fatty acids.

To construct an expression vector encoding B. subtilis enoyl-CoA reductase (encoded by fabI (SEQ ID NO: 92)), B. subtilis genomic DNA was prepared from B. subtilis strain 168 (Bacillus Genetic Stock Center, Columbus, Ohio) by picking an isolated colony from a Luria agar plate, suspending the colony in 41 μL of sterile Milli-Q water, and directly amplifying using a PCR reaction with gene-specific primers. To generate an expression plasmid not encoding a polyhistidine tag, B. subtilis fabI was amplified from the genomic DNA sample by PCR using primers (SEQ ID NO: 87 and SEQ ID NO: 88), which incorporated flanking restriction sites for NcoI and PstI into the amplified DNA (SEQ ID NO: 89). To generate an expression plasmid where fabI would be fused to a polyhistidine tag, B. subtilis fabI was amplified from the genomic DNA sample by PCR using primers (SEQ ID NO: 91 and SEQ ID NO: 88), which incorporated flanking restriction sites for XhoI and PstI into the amplified DNA (SEQ ID NO: 90).

PCR was run on samples having 41 μl of water and one suspended colony of B. subtilis 168, 1.5 μl of a 10 μM stock of each primer, 5 μl of 10×Pfx reaction mix (Invitrogen Carlsbad, Calif.), and 0.5 μl of Pfx DNA polymerase (1.25 units). PCR conditions were as follows: the samples were initially incubated at 95° C. for three minutes, followed by 30 cycles at 95° C. for 30 seconds (strand separation), 58° C. for 30 seconds (primer annealing), and 68° C. primer extension for 1.5 minutes. Following these cycles, there was a ten minute incubation at 68° C., and the samples were then held at 4° C.

The PCR products were purified using a QIAquick® PCR Purification Kit (Qiagen), double digested with restriction enzymes XhoI/PstI or NcoI/PstI, and ligated (Fast-Link Epicentre Biotechnologies, Madison, Wis.) into XhoI/PstI or NcoI/PstI-digested pTrc/His A (Invitrogen, Carlsbad, Calif.). The ligation mix was used to transform E. coli DH5α™ (Invitrogen Carlsbad, Calif.). Isolated colonies were screened by PCR using a sterile toothpick stab as an inoculum into a reaction tube containing only water, followed by addition of PCR reaction cocktail (AccuPrime™ SuperMixII, Invitrogen Carlsbad, Calif.) and primers as described above.

Recombinant plasmids were isolated and purified using the QIAPrep® Spin Miniprep Kit (Qiagen) and characterized by restriction enzyme digestion (XhoI+PstI, NcoI+PstI, DraI, MfeI, and HaeII (Invitrogen, Carlsbad, Calif. or New England Biolabs, Beverly, Mass.)). The plasmids were subsequently used to transform chemically competent BL21 STAR (DE3) (Invitrogen, Carlsbad, Calif.). Transformants were selected on Luria agar plates containing 100 μg/ml ampicillin. Plasmid DNA was isolated and purified using the QIAfilter™ Plasmid Midi Kit (Qiagen). DNA sequencing confirmed that the fabI inserts had been cloned and that the inserts encoded the FabI amino acid sequence (SEQ ID NO: 89). The resulting plasmid lacking a polyhistidine tag was designated pTrc Bs_fabI- and the plasmid incorporating a polyhistidine tag was designated pTrc Bs_fabI+.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method for producing anteiso fatty acid, the method comprising culturing a cell comprising at least one exogenous or overexpressed polynucleotide comprising a nucleic acid sequence encoding a polypeptide that catalyzes at least one of the following reactions:
(aa) conversion of pyruvate to citramalate;
(bb) conversion of citramalate to citraconate;
(cc) conversion of citraconate to β-methyl-D-malate;
(dd) conversion of β-methyl-D-malate to 2-oxobutanoate; or
(ee) conversion of threonine to 2-oxobutanoate
under conditions allowing expression of the polynucleotide(s) and production of anteiso fatty acid, wherein the cell produces more anteiso fatty acids than an otherwise similar cell that does not comprise the polynucleotide(s).

2. The method of claim 1, further comprising extracting from the culture the anteiso fatty acid or a product derived from anteiso fatty acid.

3. The method of claim 1, wherein the cell further comprises at least one exogenous or overexpressed polynucleotide comprising a nucleic acid sequence encoding a polypeptide that catalyzes at least one of the following reactions:
(ff) conversion of 2-oxobutanoate to 2-aceto-2-hydroxy-butyrate,
(gg) conversion of 2-aceto-2-hydroxy-butyrate to 2,3-dihydroxy-3-methylvalerate, or
(hh) conversion of 2,3-dihydroxy-3-methylvalerate to 2-keto-3-methylvalerate, and, optionally, the cell is modified to attenuate branched-chain amino acid aminotransferase activity.

4. The method of claim 3, wherein the cell comprises exogenous or overexpressed polynucleotides encoding polypeptides that catalyze reactions (aa), (bb), (cc), (dd), and (ff).

5. The method of claim 4, wherein the cell comprises an exogenous polynucleotide encoding a citramalate synthase, an exogenous or overexpressed polynucleotide encoding an acetohydroxy acid synthase, an exogenous or overexpressed polynucleotide encoding an isopropylmalate isomerase, and an exogenous or overexpressed polynucleotide encoding an isopropylmalate dehydrogenase.

6. The method of claim 5, wherein the citramalate synthase is CimA derived from M. jannaschii, the isopropylmalate isomerase is E. coli LeuCD, the isopropylmalate dehydrogenase is E. coli LeuB, and/or the acetohydroxy acid synthase is E. coli IlvIH, E. coli IlvIH (G14D), E. coli IlvGM, or B. subtilis IlvBH.

7. The method of claim 3, wherein the cell comprises exogenous or overexpressed polynucleotides encoding polypeptides that catalyze reactions (ee) and (ff).

8. The method of claim 7, wherein the cell comprises an exogenous or overexpressed polynucleotide encoding a threonine deaminase and an exogenous or overexpressed polynucleotide encoding an acetohydroxy acid synthase.

9. The method of claim 8, wherein the threonine deaminase is E. coli TdcB and/or the acetohydroxy acid synthase is E. coli IlvIH, E. coli IlvIH (G14D), E. coli IlvGM, or B. subtilis IlvBH.

10. The method of claim 3, wherein the one or more of the exogenous or overexpressed polynucleotides
(i) comprise a nucleic acid sequence having at least about 90 percent identity to the nucleic acid sequence set forth in SEQ ID NO: 32, 36, 42, 43, 46, 51, 57, 62, 68, or 83, or
(ii) encode a polypeptide comprising an amino acid sequence having at least about 90 percent identity to the amino acid sequence set forth in SEQ ID NO: 33, 39, 40, 41, 47, 48, 52, 53, 58, 65, 66, 67, 84, or 85.

11. The method of claim 3, wherein the cell further comprises an exogenous or overexpressed polynucleotide comprising a nucleic acid sequence encoding a branched-chain amino acid aminotransferase, an exogenous or overexpressed polynucleotide comprising a nucleic acid sequence encoding a branched-chain α-keto acid dehydrogenase, an exogenous or overexpressed polynucleotide comprising a nucleic acid sequence encoding an acyl transferase, an exogenous or overexpressed polynucleotide comprising a nucleic acid sequence encoding a 3-ketoacyl-ACP synthase, an exogenous or overexpressed polynucleotide comprising a nucleic acid sequence encoding an enoyl-ACP reductase, an exogenous or overexpressed polynucleotide comprising a nucleic acid sequence encoding a thioesterase, or a combination thereof.

12. The method of claim 11, wherein one or more of the exogenous or overexpressed polynucleotides comprise a nucleic acid sequence (i) having at least 90 percent identity to the nucleic acid sequence set forth in SEQ ID NO: 1, 4, 7, 13, 17, 18, 19, 20, 21, 22, 23, 68, 77, or 78 (ii) encoding a polypeptide having an amino acid sequence having at least 90 percent identity to the amino acid sequence set forth in SEQ ID NO: 10, 16, 24, 25, 26, 27, 28, 29, or 73.

13. The method of claim 11, wherein the cell comprises an exogenous or overexpressed polynucleotide comprising a nucleic acid sequence encoding a branched-chain α-keto acid dehydrogenase, an exogenous or overexpressed polynucleotide comprising a nucleic acid sequence encoding a 3-ketoacyl-ACP synthase, and, optionally, an exogenous or overexpressed polynucleotide comprising a nucleic acid sequence encoding a thioesterase.

14. The method of claim 13, wherein the cell is an Escherichia cell.

15. The method of claim 13, wherein the branched-chain α-keto acid dehydrogenase is B. subtilis Bkd and/or the 3-ketoacyl-ACP synthase is B. subtilis FabH.

16. A cell comprising:
(i) an exogenous or overexpressed polynucleotide comprising a nucleic acid sequence encoding a threonine deaminase or an exogenous or overexpressed polynucleotide encoding citramalate synthase;
(ii) an exogenous or overexpressed polynucleotide comprising a nucleic acid sequence encoding a branched-chain α-keto acid dehydrogenase; and
(iii) an exogenous or overexpressed polynucleotide comprising a nucleic acid sequence encoding a 3-ketoacyl-ACP synthase,
wherein the polynucleotides are expressed and the cell produces more anteiso fatty acid than an otherwise similar cell that does not comprise the polynucleotide(s).

17. The cell of claim 16, wherein (i) the threonine deaminase is E. coli TdcB or the citramalate synthase is CimA derived from M. jannaschii, (ii) the branched-chain α-keto acid dehydrogenase is B. subtilis Bkd, and (iii) the 3-ketoacyl-ACP synthase is B. subtilis FabH.

18. The cell of claim 16 further comprising an exogenous or overexpressed polynucleotide comprising a nucleic acid sequence encoding an isopropylmalate isomerase, an exogenous or overexpressed polynucleotide comprising a nucleic acid sequence encoding an isopropylmalate dehydrogenase, an exogenous or overexpressed polynucleotide comprising a nucleic acid sequence encoding an acetohydroxy acid synthase, an exogenous or overexpressed polynucleotide comprising a nucleic acid sequence encoding an enoyl-ACP synthase, an exogenous or overexpressed polynucleotide comprising a nucleic acid sequence encoding a thioesterase, or a combination thereof.

19. The cell of claim 18, wherein the isopropylmalate isomerase is E. coli LeuCD, the isopropylmalate dehydrogenase is E. coli LeuB, and/or the acetohydroxy acid synthase is E. coli IlvIH, E. coli IlvIH (G14D), E. coli IlvGM, or B. subtilis IlvBH.

20. The cell of claim 18, wherein the cell is an Escherichia cell.


SEQUENCE LISTING (92 sequences)

SEQ ID NO: 1 - DNA, 4842 bp, Bacillus subtilis
acagacaggagtgagtcaccatggcaactgagtatgacgtagtcattctgggcggcggtaccggcggttatgttgcggccatcagagccgctcagctcggcttaaaaacagccgttgtggaaaaggaaaaactcgggggaacatgtctgcataaaggctgtatcccgagtaaagcgctgcttagaagcgcagaggtataccggacagctcgtgaagccgatcaattcggagtggaaacggctggcgtgtccctcaactttgaaaaagtgcagcagcgtaagcaagccgttgttgataagcttgcagcgggtgtaaatcatttaatgaaaaaaggaaaaattgacgtgtacaccggatatggacgtatccttggaccgtcaatcttctctccgctgccgggaacaatttctgttgagcggggaaatggcgaagaaaatgacatgctgatcccgaaacaagtgatcattgcaacaggatcaagaccgagaatgcttccgggtcttgaagtggacggtaagtctgtactgacttcagatgaggcgctccaaatggaggagctgccacagtcaatcatcattgtcggcggaggggttatcggtatcgaatgggcgtctatgcttcatgattttggcgttaaggtaacggttattgaatacgcggatcgcatattgccgactgaagatctagagatttcaaaagaaatggaaagtcttcttaagaaaaaaggcatccagttcataacaggggcaaaagtgctgcctgacacaatgacaaaaacatcagacgatatcagcatacaagcggaaaaagacggagaaaccgttacctattctgctgagaaaatgcttgtttccatcggcagacaggcaaatatcgaaggcatcggcctagagaacaccgatattgttactgaaaatggcatgatttcagtcaatgaaagctgccaaacgaaggaatctcatatttatgcaatcggagacgtaatcggtggcctgcagttagctcacgttgcttcacatgagggaattattgctgttgagcattttgcaggtctcaatccgcatccgcttgatccgacgcttgtgccgaagtgcatttactcaagccctgaagctgccagtgtcggcttaaccgaagacgaagcaaaggcgaacgggcataatgtcaaaatcggcaagttcccatttatggcgattggaaaagcgcttgtatacggtgaaagcgacggttttgtcaaaatcgtggctgaccgagatacagatgatattctcggcgttcatatgattggcccgcatgtcaccgacatgatttctgaagcgggtcttgccaaagtgctggacgcaacaccgtgggaggtcgggcaaacgattcacccgcatccaacgctttctgaagcaattggagaagctgcgcttgccgcagatggcaaagccattcatttttaaaagcataaaggaggggcttgaatgagtacaaaccgacatcaagcactagggctgactgatcaggaagccgttgatatgtatagaaccatgctgttagcaagaaaaatcgatgaaagaatgtggctgttaaaccgttctggcaaaattccatttgtaatctcttgtcaaggacaggaagcagcacaggtaggagcggctttcgcacttgaccgtgaaatggattatgtattgccgtactacagagacatgggtgtcgtgctcgcgtttggcatgacagcaaaggacttaatgatgtccgggtttgcaaaagcagcagatccgaactcaggaggccgccagatgccgggacatttcggacaaaagaaaaaccgcattgtgacgggatcatctccggttacaacgcaagtgccgcacgcagtcggtattgcgcttgcgggacgtatggagaaaaaggatatcgcagcctttgttacattcggggaagggtcttcaaaccaaggcgatttccatgaaggggcaaactttgccgctgtccataagctgccggttattttcatgtgtgaaaacaacaaatacgcaatctcagtgccttacgataagcaagtcgcatgtgagaacatttccgaccgtgccataggctatgggatgcctggcgtaactgtgaatggaaatgatccgctggaagtttatcaagcggttaaagaagcacgcgaaagggcacgcagaggagaaggcccgacattaattgaaacgatttcttaccgccttacaccacattccagtgatgacgatgacagcagctacagaggccgtgaagaagtagaggaagcgaaaaaaagtgatcccctgcttacttatcaagcttacttaaaggaaacaggcctgctgtccgatgagatagaacaaaccatgctggatgaaattatggcaatcgtaaatgaagcgacggatgaagcggagaacgccccatatgcagctcctgagtcagcgcttgattatgtttatgcgaagtagggaggaagaacaaatgtcagtaatgtcatatattgatgcaatcaatttggcgatgaaagaagaaatggaacgagattctcgcgttttcgtccttggggaagatgtaggaagaaaaggcggtgtgtttaaagcgacagcgggactctatgaacaatttggggaagagcgcgttatggatacgccgcttgctgaatctgcaatcgcaggagtcggtatcggagcggcaatgtacggaatgagaccgattgctgaaatgcagtttgctgatttcattatgccggcagtcaaccaaattatttctgaagcggctaaaatccgctaccgcagcaacaatgactggagctgtccgattgtcgtcagagcgccatacggcggaggcgtgcacggagccctgtatcattctcaatcagtcgaagcaattttcgccaaccagcccggactgaaaattgtcatgccatcaacaccatatgacgcgaaagggctcttaaaagccgcagttcgtgacgaagaccccgtgctgttttttgagcacaagcgggcataccgtctgataaagggcgaggttccggctgatgattatgtcctgccaatcggcaaggcggacgtaaaaagggaaggcgacgacatcacagtgatcacatacggcctgtgtgtccacttcgccttacaagctgcagaacgtctcgaaaaagatggcatttcagcgcatgtggtggatttaagaacagtttacccgcttgataaagaagccatcatcgaagctgcgtccaaaactggaaaggttcttttggtcacagaagatacaaaagaaggcagcatcatgagcgaagtagccgcaattatatccgagcattgtctgttcgacttagacgcgccgatcaaacggcttgcaggtcctgatattccggctatgccttatgcgccgacaatggaaaaatactttatggtcaaccctgataaagtggaagcggcgatgagagaattagcggagttttaaagacgtaagggaggatacaatcatggcaattgaacaaatgacgatgccgcagcttggagaaagcgtaacagaggggacgatcagcaaatggcttgtcgcccccggtgataaagtgaacaaatacgatccgatcgcggaagtcatgacagataaggtaaatgcagaggttccgtcttcttttactggtacgataacagagcttgtgggagaagaaggccaaaccctgcaagtcggagaaatgatttgcaaaattgaaacagaaggcgcgaatccggctgaacaaaaacaagaacagccagcagcatcagaagccgctgagaaccctgttgcaaaaagtgctggagcagccgatcagcccaataaaaagcgctactcgccagctgttctccgtttggccggagagcacggcattgacctcgatcaagtgacaggaactggtgccggcgggcgcatcacacgaaaagatattcagcgcttaattgaaacaggcggcgtgcaagaacagaatcctgaggagctgaaaacagcagctcctgcaccgaagtctgcatcaaaacctgagccaaaagaagagacgtcatatcctgcgtctgcagccggtgataaagaaatccctgtcacaggtgtaagaaaagcaattgcttccaatatgaagcgaagcaaaacagaaattccgcatgcttggacgatgatggaagtcgacgtcacaaatatggttgcatatcgcaacagtataaaagattcttttaagaagacagaaggctttaatttaacgttcttcgccttttttgtaaaagcggtcgctcaggcgttaaaagaattcccgcaaatgaatagcatgtgggcgggggacaaaattattcagaaaaaggatatcaatatttcaattgcagttgccacagaggattctttatttgttccggtgattaaaaacgctgatgaaaaaacaattaaaggcattgcgaaagacattaccggcctagctaaaaaagtaagagacggaaaactcactgcagatgacatgcagggaggcacgtttaccgtcaacaacacaggttcgttcgggtctgttcagtcgatgggcattatcaactaccctcaggctgcgattcttcaagtagaatccatcgtcaaacgcccggttgtcatggacaatggcatgattgctgtcagagacatggttaatctgtgcctgtcattagatcacagagtgcttgacggtctcgtgtgcggacgattcctcggacgagtgaaacaaattttagaatcgattgacgagaagacatctgtttactaaataagcaaaaagagcattttttgaagttttgtttcaaaaaatgctctttttctatgctttattattcagcgatccgtattttcatttcg

SEQ ID NO: 2 - DNA, 44 bp, Artificial Sequence
  Synthetic primer
gagcatgggcccacagacaggagtgagtcaccatggcaactgag

SEQ ID NO: 3 - DNA, 50 bp, Artificial Sequence
  Synthetic primer
gagaccacgcgtcgaaatgaaaatacggatcgctgaataataaagcatag

SEQ ID NO: 4 - DNA, 1425 bp, Escherichia coli
atggcaactgagtatgacgtagtcattctgggcggcggtaccggcggttatgttgcggccatcagagccgctcagctcggcttaaaaacagccgttgtggaaaaggaaaaactcgggggaacatgtctgcataaaggctgtatcccgagtaaagcgctgcttagaagcgcagaggtataccggacagctcgtgaagccgatcaattcggagtggaaacggctggcgtgtccctcaactttgaaaaagtgcagcagcgtaagcaagccgttgttgataagcttgcagcgggtgtaaatcatttaatgaaaaaaggaaaaattgacgtgtacaccggatatggacgtatccttggaccgtcaatcttctctccgctgccgggaacaatttctgttgagcggggaaatggcgaagaaaatgacatgctgatcccgaaacaagtgatcattgcaacaggatcaagaccgagaatgcttccgggtcttgaagtggacggtaagtctgtactgacttcagatgaggcgctccaaatggaggagctgccacagtcaatcatcattgtcggcggaggggttatcggtatcgaatgggcgtctatgcttcatgattttggcgttaaggtaacggttattgaatacgcggatcgcatattgccgactgaagatctagagatttcaaaagaaatggaaagtcttcttaagaaaaaaggcatccagttcataacaggggcaaaagtgctgcctgacacaatgacaaaaacatcagacgatatcagcatacaagcggaaaaagacggagaaaccgttacctattctgctgagaaaatgcttgtttccatcggcagacaggcaaatatcgaaggcatcggcctagagaacaccgatattgttactgaaaatggcatgatttcagtcaatgaaagctgccaaacgaaggaatctcatatttatgcaatcggagacgtaatcggtggcctgcagttagctcacgttgcttcacatgagggaattattgctgttgagcattttgcaggtctcaatccgcatccgcttgatccgacgcttgtgccgaagtgcatttactcaagccctgaagctgccagtgtcggcttaaccgaagacgaagcaaaggcgaacgggcataatgtcaaaatcggcaagttcccatttatggcgattggaaaagcgcttgtatacggtgaaagcgacggttttgtcaaaatcgtggctgaccgagatacagatgatattctcggcgttcatatgattggcccgcatgtcaccgacatgatttctgaagcgggtcttgccaaagtgctggacgcaacaccgtgggaggtcgggcaaacgattcacccgcatccaacgctttctgaagcaattggagaagctgcgcttgccgcagatggcaaagccattcatttttaa

SEQ ID NO: 5 - DNA, 38 bp, Artificial Sequence
  Synthetic primer
gagaccatggctaaagctggaatacttggtgttggacg

SEQ ID NO: 6 - DNA, 30 bp, Artificial Sequence
  Synthetic primer
cgctcctgcagtcttgtgtgcacctcacct

SEQ ID NO: 7 - DNA, 939 bp, Bacillus subtilis
atgaaagctggaatacttggtgttggacgttacattcctgagaaggttttaacaaatcatgatcttgaaaaaatggttgaaacttctgacgagtggattcgtacaagaacaggaatagaagaaagaagaatcgcagcagatgatgtgttttcatcacatatggctgttgcagcagcgaaaaatgcgctggaacaagctgaagtggctgctgaggatctggatatgatcttggttgcaactgttacacctgatcagtcattccctacggtctcttgtatgattcaagaacaactcggcgcgaagaaagcgtgtgctatggatatcagcgcggcttgtgcgggcttcatgtacggggttgtaaccggtaaacaatttattgaatccggaacctacaagcatgttctagttgttggtgtagagaagctctcaagcattaccgactgggaagaccgcaatacagccgttctgtttggagacggagcaggcgctgcggtagtcgggccagtcagtgatgacagaggaatcctttcatttgaactaggagccgacggcacaggcggtcagcacttgtatctgaatgaaaaacgacatacaatcatgaatggacgagaagttttcaaatttgcagtccgccaaatgggagaatcatgcgtaaatgtcattgaaaaagccggactttcaaaagaggatgtcgactttttgattccgcatcaggcgaacatccgtatcatggaagctgctcgcgagcgtttagagcttcctgtcgaaaagatgtctaaaactgttcataaatatggaaatacttctgccgcatccattccgatctctcttgtagaagaattggaagccggtaaaatcaaagacggcgatgtggtcgttatggtagggttcggcggaggactaacatggggcgccattgcaatccgctggggccgataa

SEQ ID NO: 8 - DNA, 38 bp, Artificial Sequence
  Synthetic primer
gagacgctcgagatgaaagctggaatacttggtgttgg

SEQ ID NO: 9 - DNA, 30 bp, Artificial Sequence; identical to SEQ ID NO: 6
  Synthetic primer

SEQ ID NO: 10 - protein, 312 aa, Bacillus subtilis
MKAGILGVGRYIPEKVLTNHDLEKMVETSDEWIRTRTGIEERRIAADDVFSSHMAVAAAKNALEQAEVAAEDLDMILVATVTPDQSFPTVSCMIQEQLGAKKACAMDISAACAGFMYGVVTGKQFIESGTYKHVLVVGVEKLSSITDWEDRNTAVLFGDGAGAAVVGPVSDDRGILSFELGADGTGGQHLYLNEKRHTIMNGREVFKFAVRQMGESCVNVIEKAGLSKEDVDFLIPHQANIRIMEAARERLELPVEKMSKTVHKYGNTSAASIPISLVEELEAGKIKDGDVVVMVGFGGGLTWGAIAIRWGR

SEQ ID NO: 11 - DNA, 36 bp, Artificial Sequence
  Synthetic primer
gagaccatggcaaaagcaaaaattacagctatcggc

SEQ ID NO: 12 - DNA, 32 bp, Artificial Sequence
  Synthetic primer
gctcctgcagggaagaaacatcagaagaacag

SEQ ID NO: 13 - DNA, 978 bp, Bacillus subtilis
atgtcaaaagcaaaaattacagctatcggcacctatgcgccgagcagacgtttaaccaatgcagatttagaaaagatcgttgatacctctgatgaatggatcgttcagcgcacaggaatgagagaacgccggattgcggatgaacatcaatttacctctgatttatgcatagaagcggtgaagaatctcaagagccgttataaaggaacgcttgatgatgtcgatatgatcctcgttgccacaaccacatccgattacgcctttccgagtacggcatgccgcgtacaggaatatttcggctgggaaagcaccggcgcgctggatattaatgcgacatgcgccgggctgacatacggcctccatttggcaaatggattgatcacatctggccttcatcaaaaaattctcgtcatcgccggagagacgttatcaaaggtaaccgattataccgatcgaacgacatgcgtactgttcggcgatgccgcgggtgcgctgttagtagaacgagatgaagagacgccgggatttcttgcgtctgtacaaggaacaagcgggaacggcggcgatattttgtatcgtgccggactgcgaaatgaaataaacggtgtgcagcttgtcggttccggaaaaatggtgcaaaacggacgcgaggtatataaatgggccgcaagaaccgtccctggcgaatttgaacggcttttacataaagcaggactcagctccgatgatctcgattggtttgttcctcacagcgccaacttgcgcatgatcgagtcaatttgtgaaaaaacaccgttcccgattgaaaaaacgctcactagtgttgagcactacggaaacacgtcttcggtttcaattgttttggcgctcgatctcgcagtgaaagccgggaagctgaaaaaagatcaaatcgttttgcttttcgggtttggcggcggattaacctatacaggattgcttattaaatgggggatgtaa

SEQ ID NO: 14 - DNA, 41 bp, Artificial Sequence
  Synthetic primer
gagacgctcgagatgtcaaaagcaaaaattacagctatcgg

SEQ ID NO: 15 - DNA, 35 bp, Artificial Sequence
  Synthetic primer
gctcctgcaggaaggaagaaacatcagaagaacag

SEQ ID NO: 16 - protein, 325 aa, Bacillus subtilis
MSKAKITAIGTYAPSRRLTNADLEKIVDTSDEWIVQRTGMRERRIADEHQFTSDLCIEAVKNLKSRYKGTLDDVDMILVATTTSDYAFPSTACRVQEYFGWESTGALDINATCAGLTYGLHLANGLITSGLHQKILVIAGETLSKVTDYTDRTTCVLFGDAAGALLVERDEETPGFLASVQGTSGNGGDILYRAGLRNEINGVQLVGSGKMVQNGREVYKWAARTVPGEFERLLHKAGLSSDDLDWFVPHSANLRMIESICEKTPFPIEKTLTSVEHYGNTSSVSIVLALDLAVKAGKLKKDQIVLLFGFGGGLTYTGLLIKWGM

SEQ ID NO: 17 - DNA, 756 bp, Anas platyrhynchos
atggataaagttattgcacgtccgtataaacggcctaatgcgttatgcagattgatttgttttccgtgggcaggcggaaactgctcatttttcatcagatggtgtgaagcgttttcaagcattatcgttgtgtctgtgattcgccttgctggcagagaatgccgcgatacagaaccgtttcctgaagatatggctgaagtcgtaaatgaaatcacgaacgccctgcttaaagatttgcaagaaaaaccgtttgcattgtttggccatagctttggatcttttgtcagctatgcactggcggtacatcttaaagaaaaacatggattagaaccggtccacatgtttttctcaggcagctatggccctcattctgaatactttcatttgatgtacaaattgccggaagtagaagattcacgcttattggaactgattcatacacttggcggaacgccgcctgaatttttgcaaaacgaacagatcacaaaacatctgcttcgtgttctgaaagaagatcagaaagttcttgtgacgtatccttggcatgatgtgcggaaaaaatacttttcttgcgatctgacatgttttaatggctcagatgagaaaaatcatggctcagaagcatggattgccatcacatcaggcgatacgagcatctactctttaccgggaaaccatttttacttgatggaaccttcaaacgaaacatttctgatcaaatacatcacgaaatgtattgaaaacagcgatatttaa

SEQ ID NO: 18 - DNA, 756 bp, Anas platyrhynchos
atggataaggtgattgcccgtccatacaaaaggccaaatgctctctgtaggctgatttgctttccctgggctggaggtaactgttctttctttattcgatggtgcgaagccttcagcagcataattgtagtgtccgttataaggcttgctggaagagaatgtcgtgatacggagccttttccagaagacatggcagaagtagttaatgaaattacaaatgctttgttaaaagatctgcaagaaaaaccatttgcattatttggtcacagttttggatctttcgttagttatgcacttgcagtacacttgaaagagaagcacggattagagccggtccatatgtttttctcagggtcatatggcccacatagtgaatattttcatctgatgtacaaattgcctgaagtagaagatagtcgcttgcttgaacttatacacacattaggaggaactcctcctgagtttctgcaaaatgaacaaatcaccaaacatttgttacgtgttttaaaagaagaccagaaagttcttgtcacatatccttggcacgatgtaagaaagaaatacttctcttgtgatcttacctgctttaacgggtctgatgaaaaaaaccatggctcagaagcctggattgcaataaccagtggagatacttccatttacagtcttcctggaaatcacttttatctaatggagccttctaacgaaactttcttgataaaatacataactaaatgtatagaaaattctgacatatga

SEQ ID NO: 19 - DNA, 792 bp, Rattus norvegicus
atggaaacagctgttaatgccaaatcaccgagaaacgaaaaagtgcttaactgcttgtaccaaaacccggatgcagtttttaaactgatttgttttccttgggctggcggaggcagcattcattttgccaaatggggacagaaaatcaacgattctttggaagtccatgcagtacgcctggcgggaagagaaacacgccttggcgaaccgtttgcgaacgatatttaccaaatcgcagatgaaatcgttacggcgctgcttcctatcatccaggataaagcatttgcatttttcggacatagctttggctcttatattgctttaatcacagccttgttgctgaaagaaaaatacaaaatggaaccgttgcatatctttgtgtcaggcgctagcgcccctcattctacatcacgtccgcaagtccctgatttgaacgaactgacggaagaacaagtacggcatcatcttttagattttggaggcacgccgaaacatttgatcgaagatcaagatgtcttgagaatgtttatccctttgctgaaagcagatgcgggagttgtgaaaaaatttatctttgataaaccgtctaaagctcttctgtcactggatattacaggatttttaggctcagaagatacgattaaagatattgaaggatggcaggatcttacaagcggcaaatttgatgtacacatgctgccgggcgatcatttttatcttatgaaacctgataacgaaaactttatcaaaaactacatcgcgaaatgcttggaactgtcatcactgacgtaa

SEQ ID NO: 20 - DNA, 792 bp, Rattus norvegicus
atggagacagcagtcaatgctaagagtcccaggaatgaaaaggttttgaactgtttgtatcaaaatcctgatgcagttttcaagctgatctgcttcccttgggcaggaggcggctccatccattttgccaagtggggccaaaagattaacgactctctggaagtgcatgctgtaagactggctggaagagaaacccgacttggagaacctttcgcaaatgacatctaccagatagctgatgaaatcgtgaccgccctgttgcccatcattcaggataaagcttttgcgttttttggccacagttttggatcctacattgctcttattactgctctgctcctaaaggagaaatacaaaatggagccgctgcatatttttgtatccggtgcatccgcccctcactcaacatcccggcctcaagttcctgatcttaacgaattgacagaagaacaagtcagacatcaccttctggatttcggaggcacgcccaagcatctcatagaagaccaggatgttctgaggatgttcattcctttgctgaaggcagatgctggcgttgtgaaaaaattcatctttgacaagccctccaaagctcttctctctctggacataacgggcttccttggatctgaagatacaataaaggacatagaaggctggcaagacctaaccagtgggaagtttgatgtccacatgctgccaggcgaccacttttatctgatgaagcccgacaacgagaactttatcaagaactacatagccaagtgcttggaactctcgtcactcacttga

SEQ ID NO: 21 - DNA, 993 bp, Bacillus subtilis
atgagtacaaaccgacatcaagcactagggctgactgatcaggaagccgttgatatgtatagaaccatgctgttagcaagaaaaatcgatgaaagaatgtggctgttaaaccgttctggcaaaattccatttgtaatctcttgtcaaggacaggaagcagcacaggtaggagcggctttcgcacttgaccgtgaaatggattatgtattgccgtactacagagacatgggtgtcgtgctcgcgtttggcatgacagcaaaggacttaatgatgtccgggtttgcaaaagcagcagatccgaactcaggaggccgccagatgccgggacatttcggacaaaagaaaaaccgcattgtgacgggatcatctccggttacaacgcaagtgccgcacgcagtcggtattgcgcttgcgggacgtatggagaaaaaggatatcgcagcctttgttacattcggggaagggtcttcaaaccaaggcgatttccatgaaggggcaaactttgccgctgtccataagctgccggttattttcatgtgtgaaaacaacaaatacgcaatctcagtgccttacgataagcaagtcgcatgtgagaacatttccgaccgtgccataggctatgggatgcctggcgtaactgtgaatggaaatgatccgctggaagtttatcaagcggttaaagaagcacgcgaaagggcacgcagaggagaaggcccgacattaattgaaacgatttcttaccgccttacaccacattccagtgatgacgatgacagcagctacagaggccgtgaagaagtagaggaagcgaaaaaaagtgatcccctgcttacttatcaagcttacttaaaggaaacaggcctgctgtccgatgagatagaacaaaccatgctggatgaaattatggcaatcgtaaatgaagcgacggatgaagcggagaacgccccatatgcagctcctgagtcagcgcttgattatgtttatgcgaagtag

SEQ ID NO: 22 - DNA, 984 bp, Bacillus subtilis
atgtcagtaatgtcatatattgatgcaatcaatttggcgatgaaagaagaaatggaacgagattctcgcgttttcgtccttggggaagatgtaggaagaaaaggcggtgtgtttaaagcgacagcgggactctatgaacaatttggggaagagcgcgttatggatacgccgcttgctgaatctgcaatcgcaggagtcggtatcggagcggcaatgtacggaatgagaccgattgctgaaatgcagtttgctgatttcattatgccggcagtcaaccaaattatttctgaagcggctaaaatccgctaccgcagcaacaatgactggagctgtccgattgtcgtcagagcgccatacggcggaggcgtgcacggagccctgtatcattctcaatcagtcgaagcaattttcgccaaccagcccggactgaaaattgtcatgccatcaacaccatatgacgcgaaagggctcttaaaagccgcagttcgtgacgaagaccccgtgctgttttttgagcacaagcgggcataccgtctgataaagggcgaggttccggctgatgattatgtcctgccaatcggcaaggcggacgtaaaaagggaaggcgacgacatcacagtgatcacatacggcctgtgtgtccacttcgccttacaagctgcagaacgtctcgaaaaagatggcatttcagcgcatgtggtggatttaagaacagtttacccgcttgataaagaagccatcatcgaagctgcgtccaaaactggaaaggttcttttggtcacagaagatacaaaagaaggcagcatcatgagcgaagtagccgcaattatatccgagcattgtctgttcgacttagacgcgccgatcaaacggcttgcaggtcctgatattccggctatgccttatgcgccgacaatggaaaaatactttatggtcaaccctgataaagtggaagcggcgatgagagaattagcggagttttaa

SEQ ID NO: 23 - DNA, 1275 bp, Bacillus subtilis
atggcaattgaacaaatgacgatgccgcagcttggagaaagcgtaacagaggggacgatcagcaaatggcttgtcgcccccggtgataaagtgaacaaatacgatccgatcgcggaagtcatgacagataaggtaaatgcagaggttccgtcttcttttactggtacgataacagagcttgtgggagaagaaggccaaaccctgcaagtcggagaaatgatttgcaaaattgaaacagaaggcgcgaatccggctgaacaaaaacaagaacagccagcagcatcagaagccgctgagaaccctgttgcaaaaagtgctggagcagccgatcagcccaataaaaagcgctactcgccagctgttctccgtttggccggagagcacggcattgacctcgatcaagtgacaggaactggtgccggcgggcgcatcacacgaaaagatattcagcgcttaattgaaacaggcggcgtgcaagaacagaatcctgaggagctgaaaacagcagctcctgcaccgaagtctgcatcaaaacctgagccaaaagaagagacgtcatatcctgcgtctgcagccggtgataaagaaatccctgtcacaggtgtaagaaaagcaattgcttccaatatgaagcgaagcaaaacagaaattccgcatgcttggacgatgatggaagtcgacgtcacaaatatggttgcatatcgcaacagtataaaagattcttttaagaagacagaaggctttaatttaacgttcttcgccttttttgtaaaagcggtcgctcaggcgttaaaagaattcccgcaaatgaatagcatgtgggcgggggacaaaattattcagaaaaaggatatcaatatttcaattgcagttgccacagaggattctttatttgttccggtgattaaaaacgctgatgaaaaaacaattaaaggcattgcgaaagacattaccggcctagctaaaaaagtaagagacggaaaactcactgcagatgacatgcagggaggcacgtttaccgtcaacaacacaggttcgttcgggtctgttcagtcgatgggcattatcaactaccctcaggctgcgattcttcaagtagaatccatcgtcaaacgcccggttgtcatggacaatggcatgattgctgtcagagacatggttaatctgtgcctgtcattagatcacagagtgcttgacggtctcgtgtgcggacgattcctcggacgagtgaaacaaattttagaatcgattgacgagaagacatctgtttactaa

SEQ ID NO: 24 - protein, 474 aa, Bacillus subtilis
MATEYDVVILGGGTGGYVAAIRAAQLGLKTAVVEKEKLGGTCLHKGCIPSKALLRSAEVYRTAREADQFGVETAGVSLNFEKVQQRKQAVVDKLAAGVNHLMKKGKIDVYTGYGRILGPSIFSPLPGTISVERGNGEENDMLIPKQVIIATGSRPRMLPGLEVDGKSVLTSDEALQMEELPQSIIIVGGGVIGIEWASMLHDFGVKVTVIEYADRILPTEDLEISKEMESLLKKKGIQFITGAKVLPDTMTKTSDDISIQAEKDGETVTYSAEKMLVSIGRQANIEGIGLENTDIVTENGMISVNESCQTKESHIYAIGDVIGGLQLAHVASHEGIIAVEHFAGLNPHPLDPTLVPKCIYSSPEAASVGLTEDEAKANGHNVKIGKFPFMAIGKALVYGESDGFVKIVADRDTDDILGVHMIGPHVTDMISEAGLAKVLDATPWEVGQTIHPHPTLSEAIGEAALAADGKAIHF

SEQ ID NO: 25 - protein, 330 aa, Bacillus subtilis
MSTNRHQALGLTDQEAVDMYRTMLLARKIDERMWLLNRSGKIPFVISCQGQEAAQVGAAFALDREMDYVLPYYRDMGVVLAFGMTAKDLMMSGFAKAADPNSGGRQMPGHFGQKKNRIVTGSSPVTTQVPHAVGIALAGRMEKKDIAAFVTFGEGSSNQGDFHEGANFAAVHKLPVIFMCENNKYAISVPYDKQVACENISDRAIGYGMPGVTVNGNDPLEVYQAVKEARERARRGEGPTLIETISYRLTPHSSDDDDSSYRGREEVEEAKKSDPLLTYQAYLKETGLLSDEIEQTMLDEIMAIVNEATDEAENAPYAAPESALDYVYAK

SEQ ID NO: 26 - protein, 327 aa, Bacillus subtilis
MSVMSYIDAINLAMKEEMERDSRVFVLGEDVGRKGGVFKATAGLYEQFGEERVMDTPLAESAIAGVGIGAAMYGMRPIAEMQFADFIMPAVNQIISEAAKIRYRSNNDWSCPIVVRAPYGGGVHGALYHSQSVEAIFANQPGLKIVMPSTPYDAKGLLKAAVRDEDPVLFFEHKRAYRLIKGEVPADDYVLPIGKADVKREGDDITVITYGLCVHFALQAAERLEKDGISAHVVDLRTVYPLDKEAIIEAASKTGKVLLVTEDTKEGSIMSEVAAIISEHCLFDLDAPIKRLAGPDIPAMPYAPTMEKYFMVNPDKVEAAMRELAEF

SEQ ID NO: 27 - protein, 424 aa, Bacillus subtilis
MAIEQMTMPQLGESVTEGTISKWLVAPGDKVNKYDPIAEVMTDKVNAEVPSSFTGTITELVGEEGQTLQVGEMICKIETEGANPAEQKQEQPAASEAAENPVAKSAGAADQPNKKRYSPAVLRLAGEHGIDLDQVTGTGAGGRITRKDIQRLIETGGVQEQNPEELKTAAPAPKSASKPEPKEETSYPASAAGDKEIPVTGVRKAIASNMKRSKTEIPHAWTMMEVDVTNMVAYRNSIKDSFKKTEGFNLTFFAFFVKAVAQALKEFPQMNSMWAGDKIIQKKDINISIAVATEDSLFVPVIKNADEKTIKGIAKDITGLAKKVRDGKLTADDMQGGTFTVNNTGSFGSVQSMGIINYPQAAILQVESIVKRPVVMDNGMIAVRDMVNLCLSLDHRVLDGLVCGRFLGRVKQILESIDEKTSVY

SEQ ID NO: 28 - protein, 251 aa, Bacillus subtilis
MDKVIARPYKRPNALCRLICFPWAGGNCSFFIRWCEAFSSIIVVSVIRLAGRECRDTEPFPEDMAEVVNEITNALLKDLQEKPFALFGHSFGSFVSYALAVHLKEKHGLEPVHMFFSGSYGPHSEYFHLMYKLPEVEDSRLLELIHTLGGTPPEFLQNEQITKHLLRVLKEDQKVLVTYPWHDVRKKYFSCDLTCFNGSDEKNHGSEAWIAITSGDTSIYSLPGNHFYLMEPSNETFLIKYITKCIENSDI

SEQ ID NO: 29 - protein, 263 aa, Bacillus subtilis
METAVNAKSPRNEKVLNCLYQNPDAVFKLICFPWAGGGSIHFAKWGQKINDSLEVHAVRLAGRETRLGEPFANDIYQIADEIVTALLPIIQDKAFAFFGHSFGSYIALITALLLKEKYKMEPLHIFVSGASAPHSTSRPQVPDLNELTEEQVRHHLLDFGGTPKHLIEDQDVLRMFIPLLKADAGVVKKFIFDKPSKALLSLDITGFLGSEDTIKDIEGWQDLTSGKFDVHMLPGDHFYLMKPDNENFIKNYIAKCLELSSLT

SEQ ID NO: 30 - DNA, 37 bp, Artificial Sequence
  Synthetic primer
gtgccatggctcatattacatacgatctgccggttgc

SEQ ID NO: 31 - DNA, 41 bp, Artificial Sequence
  Synthetic primer
gatcgaattcatccttaggcgtcaacgaaaccggtgatttg

SEQ ID NO: 32 - DNA, 993 bp, Artificial Sequence
  Synthetic nucleotide
atggctcatattacatacgatctgccggttgctattgatgacattattgaagcgaaacaacgactggctgggcgaatttataaaacaggcatgcctcgctccaactattttagtgaacgttgcaaaggtgaaatattcctgaagtttgaaaatatgcagcgtacgggttcatttaaaattcgtggcgcatttaataaattaagttcactgaccgatgcggaaaaacgcaaaggcgtggtggcctgttctgcgggcaaccatgcgcaaggggtttccctctcctgcgcgatgctgggtatcgacggtaaagtggtgatgccaaaaggtgcgccaaaatccaaagtagcggcaacgtgcgactactccgcagaagtcgttctgcatggtgataacttcaacgacactatcgctaaagtgagcgaaattgtcgaaatggaaggccgtatttttatcccaccttacgatgatccgaaagtgattgctggccagggaacgattggtctggaaattatggaagatctctatgatgtcgataacgtgattgtgccaattggtggtggcggtttaattgctggtattgcggtggcaattaaatctattaacccgaccattcgtgttattggcgtacagtctgaaaacgttcacggcatggcggcttctttccactccggagaaataaccacgcaccgaactaccggcaccctggcggatggttgtgatgtctcccgcccgggtaatttaacttacgaaatcgttcgtgaattagtcgatgacatcgtgctggtcagcgaagacgaaatcagaaacagtatgattgccttaattcagcgcaataaagtcgtcaccgaaggcgcaggcgctctggcatgtgctgcattattaagcggtaaattagaccaatatattcaaaacagaaaaaccgtcagtattatttccggcggcaatatcgatctttctcgcgtctctcaaatcaccggtttcgttgacgcctaa

SEQ ID NO: 33 - protein, 330 aa, Artificial Sequence
  Synthetic polynucleotide
MAHITYDLPVAIDDIIEAKQRLAGRIYKTGMPRSNYFSERCKGEIFLKFENMQRTGSFKIRGAFNKLSSLTDAEKRKGVVACSAGNHAQGVSLSCAMLGIDGKVVMPKGAPKSKVAATCDYSAEVVLHGDNFNDTIAKVSEIVEMEGRIFIPPYDDPKVIAGQGTIGLEIMEDLYDVDNVIVPIGGGGLIAGIAVAIKSINPTIRVIGVQSENVHGMAASFHSGEITTHRTTGTLADGCDVSRPGNLTYEIVRELVDDIVLVSEDEIRNSMIALIQRNKVVTEGAGALACAALLSGKLDQYIQNRKTVSIISGGNIDLSRVSQITGFVDA

SEQ ID NO: 34 - DNA, 1725 bp, Escherichia coli
atggagatgttgtctggagccgagatggtcgtccgatcgcttatcgatcagggcgttaaacaagtattcggttatcccggaggcgcagtccttgatatttatgatgcattgcataccgtgggtggtattgatcatgtattagttcgtcatgagcaggcggcggtgcatatggccgatggcctggcgcgcgcgaccggggaagtcggcgtcgtgctggtaacgtcgggtccaggggcgaccaatgcgattactggcatcgccaccgcttatatggattccattccattagttgtcctttccgggcaggtagcgacctcgttgataggttacgatgcctttcaggagtgcgacatggtggggatttcgcgaccggtggttaaacacagttttctggttaagcaaacggaagacattccgcaggtgctgaaaaaggctttctggctggcggcaagtggtcgcccaggaccagtagtcgttgatttaccgaaagatattcttaatccggcgaacaaattaccctatgtctggccggagtcggtcagtatgcgttcttacaatcccactactaccggacataaagggcaaattaagcgtgctctgcaaacgctggtagcggcaaaaaaaccggttgtctacgtaggcggtggggcaatcacggcgggctgccatcagcagttgaaagaaacggtggaggcgttgaatctgcccgttgtttgctcattgatggggctgggggcgtttccggcaacgcatcgtcaggcactgggcatgctgggaatgcacggtacctacgaagccaatatgacgatgcataacgcggatgtgattttcgccgtcggggtacgatttgatgaccgaacgacgaacaatctggcaaagtactgcccaaatgccactgttctgcatatcgatattgatcctacttccatttctaaaaccgtgactgcggatatcccgattgtgggggatgctcgccaggtcctcgaacaaatgcttgaactcttgtcgcaagaatccgcccatcaaccactggatgagatccgcgactggtggcagcaaattgaacagtggcgcgctcgtcagtgcctgaaatatgacactcacagtgaaaagattaaaccgcaggcggtgatcgagactctttggcggttgacgaagggagacgcttacgtgacgtccgatgtcgggcagcaccagatgtttgctgcactttattatccattcgacaaaccgcgtcgctggatcaattccggtggcctcggcacgatgggttttggtttacctgcggcactgggcgtcaaaatggcgttgccagaagaaaccgtggtttgcgtcactggcgacggcagtattcagatgaacatccaggaactgtctaccgcgttgcaatacgagttgcccgtactggtggtgaatctcaataaccgctatctggggatggtgaagcagtggcaggacatgatctattccggccgtcattcacaatcttatatgcaatcgctacccgatttcgtccgtctggcggaagcctatgggcatgtcgggatccagatttctcatccgcatgagctggaaagcaaacttagcgaggcgctggaacaggtgcgcaataatcgcctggtgtttgttgatgttaccgtcgatggcagcgagcacgtctacccgatgcagattcgcgggggcggaatggatgaaatgtggttaagcaaaacggagagaacctga

SEQ ID NO: 35 - DNA, 492 bp, Escherichia coli
atgcgccggatattatcagtcttactcgaaaatgaatcaggcgcgttatcccgcgtgattggccttttttcccagcgtggctacaacattgaaagcctgaccgttgcgccaaccgacgatccgacattatcgcgtatgaccatccagaccgtgggcgatgaaaaagtacttgagcagatcgaaaagcaattacacaaactggtcgatgtcttgcgcgtgagtgagttggggcagggcgcgcatgttgagcgggaaatcatgctggtgaaaattcaggccagcggttacgggcgtgacgaagtgaaacgtaatacggaaatattccgtgggcaaattatcgatgtcacaccctcgctttataccgttcaattagcaggcaccagcggtaagcttgatgcatttttagcatcgattcgcgatgtggcgaaaattgtggaggttgctcgctctggtgtggtcggactttcgcgcggcgataaaataatgcgttga

SEQ ID NO: 36 - DNA, 2242 bp, Escherichia coli
gaattcgaggaggcaggccatggagatgttgtctggagccgagatggtcgtccgatcgcttatcgatcagggcgttaaacaagtattcggttatcccggaggcgcagtccttgatatttatgatgcattgcataccgtgggtggtattgatcatgtattagttcgtcatgagcaggcggcggtgcatatggccgatggcctggcgcgcgcgaccggggaagtcggcgtcgtgctggtaacgtcgggtccaggggcgaccaatgcgattactggcatcgccaccgcttatatggattccattccattagttgtcctttccgggcaggtagcgacctcgttgataggttacgatgcctttcaggagtgcgacatggtggggatttcgcgaccggtggttaaacacagttttctggttaagcaaacggaagacattccgcaggtgctgaaaaaggctttctggctggcggcaagtggtcgcccaggaccagtagtcgttgatttaccgaaagatattcttaatccggcgaacaaattaccctatgtctggccggagtcggtcagtatgcgttcttacaatcccactactaccggacataaagggcaaattaagcgtgctctgcaaacgctggtagcggcaaaaaaaccggttgtctacgtaggcggtggggcaatcacggcgggctgccatcagcagttgaaagaaacggtggaggcgttgaatctgcccgttgtttgctcattgatggggctgggggcgtttccggcaacgcatcgtcaggcactgggcatgctgggaatgcacggtacctacgaagccaatatgacgatgcataacgcggatgtgattttcgccgtcggggtacgatttgatgaccgaacgacgaacaatctggcaaagtactgcccaaatgccactgttctgcatatcgatattgatcctacttccatttctaaaaccgtgactgcggatatcccgattgtgggggatgctcgccaggtcctcgaacaaatgcttgaactcttgtcgcaagaatccgcccatcaaccactggatgagatccgcgactggtggcagcaaattgaacagtggcgcgctcgtcagtgcctgaaatatgacactcacagtgaaaagattaaaccgcaggcggtgatcgagactctttggcggttgacgaagggagacgcttacgtgacgtccgatgtcgggcagcaccagatgtttgctgcactttattatccattcgacaaaccgcgtcgctggatcaattccggtggcctcggcacgatgggttttggtttacctgcggcactgggcgtcaaaatggcgttgccagaagaaaccgtggtttgcgtcactggcgacggcagtattcagatgaacatccaggaactgtctaccgcgttgcaatacgagttgcccgtactggtggtgaatctcaataaccgctatctggggatggtgaagcagtggcaggacatgatctattccggccgtcattcacaatcttatatgcaatcgctacccgatttcgtccgtctggcggaagcctatgggcatgtcgggatccagatttctcatccgcatgagctggaaagcaaacttagcgaggcgctggaacaggtgcgcaataatcgcctggtgtttgttgatgttaccgtcgatggcagcgagcacgtctacccgatgcagattcgcgggggcggaatggatgaaatgtggttaagcaaaacggagagaacctgattatgcgccggatattatcagtcttactcgaaaatgaatcaggcgcgttatcccgcgtgattggccttttttcccagcgtggctacaacattgaaagcctgaccgttgcgccaaccgacgatccgacattatcgcgtatgaccatccagaccgtgggcgatgaaaaagtacttgagcagatcgaaaagcaattacacaaactggtcgatgtcttgcgcgtgagtgagttggggcagggcgcgcatgttgagcgggaaatcatgctggtgaaaattcaggccagcggttacgggcgtgacgaagtgaaacgtaatacggaaatattccgtgggcaaattatcgatgtcacaccctcgctttataccgttcaattagcaggcaccagcggtaagcttgatgcatttttagcatcgattcgcgatgtggcgaaaattgtggaggttgctcgctctggtgtggtcggactttcgcgcggcgataaaataatgcgttgaattc

SEQ ID NO: 37 - DNA, 38 bp, Artificial Sequence
  Synthetic primer
gatgaattcgaggaggcaggccatggagatgttgtctg

SEQ ID NO: 38 - DNA, 31 bp, Artificial Sequence
  Synthetic primer
ttgagaattcaacgcattattttatcgccgc

SEQ ID NO: 39 - protein, 574 aa, Escherichia coli
MEMLSGAEMVVRSLIDQGVKQVFGYPGGAVLDIYDALHTVGGIDHVLVRHEQAAVHMADGLARATGEVGVVLVTSGPGATNAITGIATAYMDSIPLVVLSGQVATSLIGYDAFQECDMVGISRPVVKHSFLVKQTEDIPQVLKKAFWLAASGRPGPVVVDLPKDILNPANKLPYVWPESVSMRSYNPTTTGHKGQIKRALQTLVAAKKPVVYVGGGAITAGCHQQLKETVEALNLPVVCSLMGLGAFPATHRQALGMLGMHGTYEANMTMHNADVIFAVGVRFDDRTTNNLAKYCPNATVLHIDIDPTSISKTVTADIPIVGDARQVLEQMLELLSQESAHQPLDEIRDWWQQIEQWRARQCLKYDTHSEKIKPQAVIETLWRLTKGDAYVTSDVGQHQMFAALYYPFDKPRRWINSGGLGTMGFGLPAALGVKMALPEETVVCVTGDGSIQMNIQELSTALQYELPVLVVNLNNRYLGMVKQWQDMIYSGRHSQSYMQSLPDFVRLAEAYGHVGIQISHPHELESKLSEALEQVRNNRLVFVDVTVDGSEHVYPMQIRGGGMDEMWLSKTERT

SEQ ID NO: 40 - protein, 163 aa, Escherichia coli
MRRILSVLLENESGALSRVIGLFSQRGYNIESLTVAPTDDPTLSRMTIQTVGDEKVLEQIEKQLHKLVDVLRVSELGQGAHVEREIMLVKIQASGYGRDEVKRNTEIFRGQIIDVTPSLYTVQLAGTSGKLDAFLASIRDVAKIVEVARSGVVGLSRGDKIMR

SEQ ID NO: 41 - protein, 163 aa, Artificial Sequence
  Synthetic polypeptide
MRRILSVLLENESDALSRVIGLFSQRGYNIESLTVAPTDDPTLSRMTIQTVGDEKVLEQIEKQLHKLVDVLRVSELGQGAHVEREIMLVKIQASGYGRDEVKRNTEIFRGQIIDVTPSLYTVQLAGTSGKLDAFLASIRDVAKIVEVARSGVVGLSRGDKIMR

SEQ ID NO: 42 - DNA, 1058 bp, Escherichia coli
gacgtccgatgtcgggcagcaccagatgtttgctgcactttattatccattcgacaaaccgcgtcgctggatcaattccggtggcctcggcacgatgggttttggtttacctgcggcactgggcgtcaaaatggcgttgccagaagaaaccgtggtttgcgtcactggcgacggcagtattcagatgaacatccaggaactgtctaccgcgttgcaatacgagttgcccgtactggtggtgaatctcaataaccgctatctggggatggtgaagcagtggcaggacatgatctattccggccgtcattcacaatcttatatgcaatcgctacccgatttcgtccgtctggcggaagcctatgggcatgtcgggatccagatttctcatccgcatgagctggaaagcaaacttagcgaggcgctggaacaggtgcgcaataatcgcctggtgtttgttgatgttaccgtcgatggcagcgagcacgtctacccgatgcagattcgcgggggcggaatggatgaaatgtggttaagcaaaacggagagaacctgattatgcgccggatattatcagtcttactcgaaaatgaatcagacgcgttatcccgcgtgattggccttttttcccagcgtggctacaacattgaaagcctgaccgttgcgccaaccgacgatccgacatatcgcgtatgaccatccagaccgtgggcgatgaaaaagtacttgagcagatcgaaaagcaattacacaaactggtcgatgtcttgcgcgtgagtgagttggggcagggcgcgcatgttgagcgggaaatcatgctggtgaaaattcaggccagcggttacgggcgtgacgaagtgaaacgtaatacggaaatattccgtgggcaaattatcgatgtcacaccctcgctttataccgttcaattagcaggcaccagcggtaagcttgatgcatttttagcatcgattcgcgatgtggcgaaaattgtggaggttgctcgctctggtgtggtcggactttcgcgcggcgataaaataatgcgttgaattcgatctaga

SEQ ID NO: 43 - DNA, 1058 bp, Escherichia coli
gacgtccgatgtcgggcagcaccagatgtttgctgcactttattatccattcgacaaaccgcgtcgctggatcaattccggtggcctcggcacgatgggttttggtttacctgcggcactgggcgtcaaaatggcgttgccagaagaaaccgtggtttgcgtcactggcgacggcagtattcagatgaacatccaggaactgtctaccgcgttgcaatacgagttgcccgtactggtggtgaatctcaataaccgctatctggggatggtgaagcagtggcaggacatgatctattccggccgtcattcacaatcttatatgcaatcgctacccgatttcgtccgtctggcggaagcctatgggcatgtcgggatccagatttctcatccgcatgagctggaaagcaaacttagcgaggcgctggaacaggtgcgcaataatcgcctggtgtttgttgatgttaccgtcgatggcagcgagcacgtctacccgatgcagattcgcgggggcggaatggatgaaatgtggttaagcaaaacggagagaacctgattatgcgccggatattatcagtcttactcgaaaatgaatcaggcgcgttatcccgcgtgattggccttttttcccagcgtggctacaacattgaaagcctgaccgttgcgccaaccgacgatccgacatatcgcgtatgaccatccagaccgtgggcgatgaaaaagtacttgagcagatcgaaaagcaattacacaaactggtcgatgtcttgcgcgtgagtgagttggggcagggcgcgcatgttgagcgggaaatcatgctggtgaaaattcaggccagcggttacgggcgtgacgaagtgaaacgtaatacggaaatattccgtgggcaaattatcgatgtcacaccctcgctttataccgttcaattagcaggcaccagcggtaagcttgatgcatttttagcatcgattcgcgatgtggcgaaaattgtggaggttgctcgctctggtgtggtcggactttcgcgcggcgataaaataatgcgttgaattcgatctaga

SEQ ID NO: 44 - DNA, 1647 bp, Escherichia coli
atgaatggcgcacagtgggtggtacatgcgttgcgggcacagggtgtgaacaccgttttcggttatccgggtggcgcaattatgccggtttacgatgcattgtatgacggcggcgtggagcacttgctatgccgacatgagcagggtgcggcaatggcggctatcggttatgctcgtgctaccggcaaaactggcgtatgtatcgccacgtctggtccgggcgcaaccaacctgataaccgggcttgcggacgcactgttagattccatccctgttgttgccatcaccggtcaagtgtccgcaccgtttatcggcactgacgcatttcaggaagtggatgtcctgggattgtcgttagcctgtaccaagcacagctttctggtgcagtcgctggaagagttgccgcgcatcatggctgaagcattcgacgttgcctgctcaggtcgtcctggtccggttctggtcgatatcccaaaagatatccagttagccagcggtgacctggaaccgtggttcaccaccgttgaaaacgaagtgactttcccacatgccgaagttgagcaagcgcgccagatgctggcaaaagcgcaaaaaccgatgctgtacgttggcggtggcgtgggtatggcgcaggcagttccggctttgcgtgaatttctcgctgccacaaaaatgcctgccacctgtacgctgaaagggctgggcgcagtagaagcagattatccgtactatctgggcatgctgggaatgcatggcaccaaagcggcgaacttcgcggtgcaggagtgcgacttgctgatcgccgtgggtgcacgttttgatgaccgggtgaccggcaaactgaacaccttcgcaccacacgccagtgttatccatatggatatcgacccggcagaaatgaacaagctgcgtcaggcacatgtggcattacaaggtgatttaaatgctctgttaccagcattacagcagccgttaaatatcaatgactggcagctacactgcgcgcagctgcgtgatgaacatgcctggcgttacgaccatcccggtgacgctatctacgcgccgttgttgttaaaacaactgtcagatcgtaaacctgcggattgcgtcgtgaccacagatgtggggcagcaccagatgtgggctgcgcagcacatcgcccacactcgcccggaaaatttcatcacctccagcggcttaggcaccatgggttttggtttaccggcggcggttggcgcgcaagtcgcgcgaccaaacgataccgtcgtctgtatctccggtgacggctctttcatgatgaatgtgcaagagctgggcaccgtaaaacgcaagcagttaccgttgaaaatcgtcttactcgataaccaacggttagggatggttcgacaatggcagcaactgtttttccaggaacgatatagcgaaaccacccttaccgataaccccgatttcctcatgttagccagcgccttcggcatccctggccaacacatcacccgtaaagaccaggttgaagcggcactcgacaccatgctgaacagtgatgggccatacctgcttcatgtctcaatcgacgaacttgagaacgtctggccgctggtgccgcctggtgccagtaattcagaaatgttggagaaattatcatga

SEQ ID NO: 45 - DNA, 261 bp, Escherichia coli
atgcaacatcaggtcaatgtatcagctcgcttcaatccggaaaccttagaacgtgttttacgcgtggtgcgtcatcgtggtttccacgtctgctcaatgaatatggccgccgccagcgatgcacaaaatataaatatcgaattgaccgttgccagcccacggtcggtcgacttactgtttagtcagttaaataaactggtggacgtcgcacacgttgccatctgccagagcacaaccacatcacaacaaatccgcgcctga

SEQ ID NO: 46 - DNA, 1938 bp, Escherichia coli
gcggccgcaggacggggaactaactatgaatggcgcacagtgggtggtacatgcgttgcgggcacagggtgtgaacaccgttttcggttatccgggtggcgcaattatgccggtttacgatgcattgtatgacggcggcgtggagcacttgctatgccgacatgagcagggtgcggcaatggcggctatcggttatgctcgtgctaccggcaaaactggcgtatgtatcgccacgtctggtccgggcgcaaccaacctgataaccgggcttgcggacgcactgttagattccatccctgttgttgccatcaccggtcaagtgtccgcaccgtttatcggcactgacgcatttcaggaagtggatgtcctgggattgtcgttagcctgtaccaagcacagctttctggtgcagtcgctggaagagttgccgcgcatcatggctgaagcattcgacgttgcctgctcaggtcgtcctggtccggttctggtcgatatcccaaaagatatccagttagccagcggtgacctggaaccgtggttcaccaccgttgaaaacgaagtgactttcccacatgccgaagttgagcaagcgcgccagatgctggcaaaagcgcaaaaaccgatgctgtacgttggcggtggcgtgggtatggcgcaggcagttccggctttgcgtgaatttctcgctgccacaaaaatgcctgccacctgtacgctgaaagggctgggcgcagtagaagcagattatccgtactatctgggcatgctgggaatgcatggcaccaaagcggcgaacttcgcggtgcaggagtgcgacttgctgatcgccgtgggtgcacgttttgatgaccgggtgaccggcaaactgaacaccttcgcaccacacgccagtgttatccatatggatatcgacccggcagaaatgaacaagctgcgtcaggcacatgtggcattacaaggtgatttaaatgctctgttaccagcattacagcagccgttaaatatcaatgactggcagctacactgcgcgcagctgcgtgatgaacatgcctggcgttacgaccatcccggtgacgctatctacgcgccgttgttgttaaaacaactgtcagatcgtaaacctgcggattgcgtcgtgaccacagatgtggggcagcaccagatgtgggctgcgcagcacatcgcccacactcgcccggaaaatttcatcacctccagcggcttaggcaccatgggttttggtttaccggcggcggttggcgcgcaagtcgcgcgaccaaacgataccgtcgtctgtatctccggtgacggctctttcatgatgaatgtgcaagagctgggcaccgtaaaacgcaagcagttaccgttgaaaatcgtcttactcgataaccaacggttagggatggttcgacaatggcagcaactgtttttccaggaacgatatagcgaaaccacccttaccgataaccccgatttcctcatgttagccagcgccttcggcatccctggccaacacatcacccgtaaagaccaggttgaagcggcactcgacaccatgctgaacagtgatgggccatacctgcttcatgtctcaatcgacgaacttgagaacgtctggccgctggtgccgcctggtgccagtaattcagaaatgttggagaaattatcatgatgcaacatcaggtcaatgtatcagctcgcttcaatccggaaaccttagaacgtgttttacgcgtggtgcgtcatcgtggtttccacgtctgctcaatgaatatggccgccgccagcgatgcacaaaatataaatatcgaattgaccgttgccagcccacggtcggtcgacttactgtttagtcagttaaataaactggtggacgtcgcacacgttgccatctgccagagcacaaccacatcacaacaaatccgcgcctgagaattc

SEQ ID NO: 47 - protein, 548 aa, Escherichia coli
MNGAQWVVHALRAQGVNTVFGYPGGAIMPVYDALYDGGVEHLLCRHEQGAAMAAIGYARATGKTGVCIATSGPGATNLITGLADALLDSIPVVAITGQVSAPFIGTDAFQEVDVLGLSLACTKHSFLVQSLEELPRIMAEAFDVACSGRPGPVLVDIPKDIQLASGDLEPWFTTVENEVTFPHAEVEQARQMLAKAQKPMLYVGGGVGMAQAVPALREFLAATKMPATCTLKGLGAVEADYPYYLGMLGMHGTKAANFAVQECDLLIAVGARFDDRVTGKLNTFAPHASVIHMDIDPAEMNKLRQAHVALQGDLNALLPALQQPLNINDWQLHCAQLRDEHAWRYDHPGDAIYAPLLLKQLSDRKPADCVVTTDVGQHQMWAAQHIAHTRPENFITSSGLGTMGFGLPAAVGAQVARPNDTVVCISGDGSFMMNVQELGTVKRKQLPLKIVLLDNQRLGMVRQWQQLFFQERYSETTLTDNPDFLMLASAFGIPGQHITRKDQVEAALDTMLNSDGPYLLHVSIDELENVWPLVPPGASNSEMLEKLS

SEQ ID NO: 48 - protein, 87 aa, Escherichia coli
MMQHQVNVSARFNPETLERVLRVVRHRGFHVCSMNMAAASDAQNINIELTVASPRSVDLLFSQLNKLVDVAHVAICQSTTTSQQIRA

SEQ ID NO: 49 - DNA, 1725 bp, Bacillus subtilis
atggggactaatgtacaggtggattcagcatctgccgaatgtacacagacgatgagcggagcattaatgctgattgaatcattaaaaaaagagaaagtagaaatgatcttcggttatccgggcggggctgtgcttccgatttacgataagctatacaattcagggttggtacatatccttccccgtcacgaacaaggagcaattcatgcagcggagggatacgcaagggtctccggaaaaccgggtgtcgtcattgccacgtcagggccgggagcgacaaaccttgttacaggccttgctgatgccatgattgattcattgccgttagtcgtctttacagggcaggtagcaacctctgtaatcgggagcgatgcatttcaggaagcagacattttagggattacgatgccagtaacaaaacacagctaccaggttcgccagccggaagatctgccgcgcatcattaaagaagcgttccatattgcaacaactggaagacccggacctgtattgattgatattccgaaagatgtagcaacaattgaaggagaattcagctacgatcatgagatgaatctcccgggataccagccgacaacagagccgaattatttgcagatccgcaagcttgtggaagccgtgagcagtgcgaaaaaaccggtgatcctggcgggtgcgggcgtactgcacggaaaagcgtcagaagaattaaaaaattatgctgaacagcagcaaatccctgtggcacacacccttttggggctcggaggcttcccggctgaccatccgcttttcctagggatggcgggaatgcacggtacttatacagccaatatggcccttcatgaatgtgatctattaatcagtatcggcgcccgttttgatgaccgtgtcacaggaaacctgaaacactttgccagaaacgcaaagatagcccacatcgatattgatccagctgaaatcggaaaaatcatgaaaacacagattcctgtagtcggagacagcaaaattgtcctgcaggagctgatcaaacaagacggcaaacaaagcgattcaagcgaatggaaaaaacagctcgcagaatggaaagaagagtatccgctctggtatgtagataatgaagaagaaggttttaaacctcagaaattgattgaatatattcatcaatttacaaaaggagaggccattgtcgcaacggatgtaggccagcatcaaatgtggtcagcgcaattttatccgttccaaaaagcagataaatgggtcacgtcaggcggacttggaacgatgggattcggtcttccggcggcgatcggcgcacagctggccgaaaaagatgctactgttgtcgcggttgtcggagacggcggattccaaatgacgcttcaagaactcgatgttattcgcgaattaaatcttccggtcaaggtagtgattttaaataacgcttgtctcggaatggtcagacagtggcaggaaattttctatgaagaacgttattcagaatctaaattcgcttctcagcctgacttcgtcaaattgtccgaagcatacggcattaaaggcatcagaatttcatcagaagcggaagcaaaggaaaagctggaagaggcattaacatcaagagaacctgttgtcattgacgtgcgggttgccagcgaagaaaaagtattcccgatggtggctccggggaaagggctgcatgaaatggtgggggtgaaaccttga

SEQ ID NO: 50 - DNA, 519 bp, Bacillus subtilis
ttgaaaagaattatcacattgactgtggtgaaccgctccggggtgttaaaccggatcaccggtctattcacaaaaaggcattacaacattgaaagcattacagttggacacacagaaacagccggcgtttccagaatcaccttcgtcgttcatgttgaaggtgaaaatgatgttgaacagttaacgaaacagctcaacaaacagattgatgtgctgaaagtcacagacatcacaaatcaatcgattgtccagagggagctggccttaatcaaggttgtctccgcaccttcaacaagaacagagattaatggaatcatagaaccgtttagagcctctgtcgttgatgtcagcagagacagcatcgttgttcaggtgacaggtgaatctaacaaaattgaagcgcttattgagttattaaaaccttatggcattaaagaaatcgcgagaacaggtacaacggcttttgcgaggggaacccagcaaaaggcgtcatccaataaaacaatatctattgtataa

SEQ ID NO: 51 - DNA, 2270 bp, Bacillus subtilis
gcggccgcaaaaggaggaactaaaatggggactaatgtacaggtggattcagcatctgccgaatgtacacagacgatgagcggagcattaatgctgattgaatcattaaaaaaagagaaagtagaaatgatcttcggttatccgggcggggctgtgcttccgatttacgataagctatacaattcagggttggtacatatccttccccgtcacgaacaaggagcaattcatgcagcggagggatacgcaagggtctccggaaaaccgggtgtcgtcattgccacgtcagggccgggagcgacaaaccttgttacaggccttgctgatgccatgattgattcattgccgttagtcgtctttacagggcaggtagcaacctctgtaatcgggagcgatgcatttcaggaagcagacattttagggattacgatgccagtaacaaaacacagctaccaggttcgccagccggaagatctgccgcgcatcattaaagaagcgttccatattgcaacaactggaagacccggacctgtattgattgatattccgaaagatgtagcaacaattgaaggagagttcagctacgatcatgagatgaatctcccgggataccagccgacaacagagccgaattatttgcagatccgcaagcttgtggaagccgtgagcagtgcgaaaaaaccggtgatcctggcgggtgcgggcgtactgcacggaaaagcgtcagaagaattaaaaaattatgctgaacagcagcaaatccctgtggcacacacccttttggggctcggaggcttcccggctgaccatccgcttttcctagggatggcgggaatgcacggtacttatacagccaatatggcccttcatgaatgtgatctattaatcagtatcggcgcccgttttgatgaccgtgtcacaggaaacctgaaacactttgccagaaacgcaaagatagcccacatcgatattgatccagctgaaatcggaaaaatcatgaaaacacagattcctgtagtcggagacagcaaaattgtcctgcaggagctgatcaaacaagacggcaaacaaagcgattcaagcgaatggaaaaaacagctcgcagaatggaaagaagagtatccgctctggtatgtagataatgaagaagaaggttttaaacctcagaaattgattgaatatattcatcaatttacaaaaggagaggccattgtcgcaacggatgtaggccagcatcaaatgtggtcagcgcaattttatccgttccaaaaagcagataaatgggtcacgtcaggcggacttggaacgatgggattcggtcttccggcggcgatcggcgcacagctggccgaaaaagatgctactgttgtcgcggttgtcggagacggcggattccaaatgacgcttcaagaactcgatgttattcgcgaattaaatcttccggtcaaggtagtgattttaaataacgcttgtctcggaatggtcagacagtggcaggaaattttctatgaagaacgttattcagaatctaaattcgcttctcagcctgacttcgtcaaattgtccgaagcatacggcattaaaggcatcagaatttcatcagaagcggaagcaaaggaaaagctggaagaggcattaacatcaagagaacctgttgtcattgacgtgcgggttgccagcgaagaaaaagtattcccgatggtggctccggggaaagggctgcatgaaatggtgggggtgaaaccttgaaaagaattatcacattgactgtggtgaaccgctccggggtgttaaaccggatcaccggtctattcacaaaaaggcattacaacattgaaagcattacagttggacacacagaaacagccggcgtttccagaatcaccttcgtcgttcatgttgaaggtgaaaatgatgttgaacagttaacgaaacagctcaacaaacagattgatgtgctgaaagtcacagacatcacaaatcaatcgattgtccagagggagctggccttaatcaaggttgtctccgcaccttcaacaagaacagagattaatggaatcatagaaccgtttagagcctctgtcgttgatgtcagcagagacagcatcgttgttcaggtgacaggtgaatctaacaaaattgaagcgcttattgagttattaaaaccttatggcattaaagaaatcgcgagaacaggtacaacggcttttgcgaggggaacccagcaaaaggcgtcatccaataaaacaatatctattgtataagaattc

SEQ ID NO: 52 - protein, 574 aa, Bacillus subtilis
MGTNVQVDSASAECTQTMSGALMLIESLKKEKVEMIFGYPGGAVLPIYDKLYNSGLVHILPRHEQGAIHAAEGYARVSGKPGVVIATSGPGATNLVTGLADAMIDSLPLVVFTGQVATSVIGSDAFQEADILGITMPVTKHSYQVRQPEDLPRIIKEAFHIATTGRPGPVLIDIPKDVATIEGEFSYDHEMNLPGYQPTTEPNYLQIRKLVEAVSSAKKPVILAGAGVLHGKASEELKNYAEQQQIPVAHTLLGLGGFPADHPLFLGMAGMHGTYTANMALHECDLLISIGARFDDRVTGNLKHFARNAKIAHIDIDPAEIGKIMKTQIPVVGDSKIVLQELIKQDGKQSDSSEWKKQLAEWKEEYPLWYVDNEEEGFKPQKLIEYIHQFTKGEAIVATDVGQHQMWSAQFYPFQKADKWVTSGGLGTMGFGLPAAIGAQLAEKDATVVAVVGDGGFQMTLQELDVIRELNLPVKVVILNNACLGMVRQWQEIFYEERYSESKFASQPDFVKLSEAYGIKGIRISSEAEAKEKLEEALTSREPVVIDVRVASEEKVFPMVAPGKGLHEMVGVKP

SEQ ID NO: 53 - protein, 172 aa, Bacillus subtilis
MKRIITLTVVNRSGVLNRITGLFTKRHYNIESITVGHTETAGVSRITFVVHVEGENDVEQLTKQLNKQIDVLKVTDITNQSIVQRELALIKVVSAPSTRTEINGIIEPFRASVVDVSRDSIVVQVTGESNKIEALIELLKPYGIKEIARTGTTAFARGTQQKASSNKTISIV

SEQ ID NO: 54 - DNA, 2393 bp, Artificial Sequence
  Synthetic nucleotide
atgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttgggctaacaggaggaattaaccatggggggttctcatcatcatcatcatcatggtatggctagcatgactggtggacagcaaatgggtcgggatctgtacgacgatgacgataaggatcgatggggatccgagctcgagatgaaagctggaatacttggtgttggacgttacattcctgagaaggttttaacaaatcatgatcttgaaaaaatggttgaaacttctgacgagtggattcgtacaagaacaggaatagaagaaagaagaatcgcagcagatgatgtgttttcatcacatatggctgttgcagcagcgaaaaatgcgctggaacaagctgaagtggctgctgaggatctggatatgatcttggttgcaactgttacacctgatcagtcattccctacggtctcttgtatgattcaagaacaactcggcgcgaagaaagcgtgtgctatggatatcagcgcggcttgtgcgggcttcatgtacggggttgtaaccggtaaacaatttattgaatccggaacctacaagcatgttctagttgttggtgtagagaagctctcaagcattaccgactgggaagaccgcaatacagccgttctgtttggagacggagcaggcgctgcggtagtcgggccagtcagtgatgacagaggaatcctttcatttgaactaggagccgacggcacaggcggtcagcacttgtatctgaatgaaaaacgacatacaatcatgaatggacgagaagttttcaaatttgcagtccgccaaatgggagaatcatgcgtaaatgtcattgaaaaagccggactttcaaaagaggatgtcgactttttgattccgcatcaggcgaacatccgtatcatggaagctgctcgcgagcgtttagagcttcctgtcgaaaagatgtctaaaactgttcataaatatggaaatacttctgccgcatccattccgatctctcttgtagaagaattggaagccggtaaaatcaaagacggcgatgtggtcgttatggtagggttcggcggaggactaacatggggcgccattgcaatccgctggggccgataaaaaaaggtgaggtgcacacaaga

SEQ ID NO: 55 - DNA, 48 bp, Artificial Sequence
  Synthetic primer
cgtattttcatttcgacgcgtatgcataatgtgcctgtcaaatggacg

SEQ ID NO: 56 - DNA, 51 bp, Artificial Sequence
  Synthetic primer
atttgatgcctctagcacgcgttcttgtgtgcacctcacctttttttatcg

SEQ ID NO: 57 - DNA, 1209 bp, Artificial Sequence
  Synthetic primer
tcatgatggtgcgcatttttgataccacgctgcgtgacggtgaacagacgccgggcgttagcctgacgccgaacgataaactggaaattgccaaaaaactggatgaactgggcgttgacgtcatcgaagccggtagcgcagtgacctctaaaggcgaacgcgaaggtattaaactgatcacgaaagaaggcctgaatgccgaaatttgctctttcgttcgtgcactgccggtcgatattgacgcggccctggaatgtgatgttgacagcgtccatctggtggttccgacctctccgatccacatgaaatataaactgcgtaaaaccgaagatgaagtgctggttacggctctgaaagcggttgaatacgccaaagaacagggtctgattgtcgaactgtcagccgaagatgcaacgcgctcggacgtgaactttctgatcaaactgttcaatgaaggcgaaaaagttggtgcagatcgtgtctgcgtgtgtgacaccgttggcgtcctgacgccgcagaaatcacaagaactgttcaagaaaattaccgaaaacgtgaatctgccggtgtcggttcattgccacaacgatttcggtatggcgaccgcaaatgcgtgcagcgcggtgctgggcggtgcggttcaatgtcatgtcacggtgaacggcatcggtgaacgcgctggcaatgcgagtctggaagaagtcgtggcagcttccaaaattctgtatggttacgataccaaaatcaaaatggaaaaactgtacgaagtcagtcgcattgtgtcccgtctgatgaaactgccggtcccgccgaacaaagctatcgtgggcgataatgcttttgcgcatgaagcgggcattcacgtggacggtctgatcaaaaacaccgaaacgtatgaaccgattaaaccggaaatggttggcaatcgtcgccgtattatcctgggcaaacactctggtcgtaaagcgctgaaatacaaactggatctgatgggtattaacgttagtgacgaacaactgaacaaaatctatgaacgtgtgaaagaatttggcgatctgggtaaatacattagcgatgccgacctgctggcaatcgtgcgtgaagttaccggtaaactgtgatgtcgaagaattaccatattgccgtattgccgggggacggtattggtccggagcggccgcttaattaagtttaaactctagagaattc

SEQ ID NO: 58 - protein, 372 aa, Artificial Sequence
  Synthetic polypeptide
MMVRIFDTTLRDGEQTPGVSLTPNDKLEIAKKLDELGVDVIEAGSAVTSKGEREGIKLITKEGLNAEICSFVRALPVDIDAALECDVDSVHLVVPTSPIHMKYKLRKTEDEVLVTALKAVEYAKEQGLIVELSAEDATRSDVNFLIKLFNEGEKVGADRVCVCDTVGVLTPQKSQELFKKITENVNLPVSVHCHNDFGMATANACSAVLGGAVQCHVTVNGIGERAGNASLEEVVAASKILYGYDTKIKMEKLYEVSRIVSRLMKLPVPPNKAIVGDNAFAHEAGIHVDGLIKNTETYEPIKPEMVGNRRRIILGKHSGRKALKYKLDLMGINVSDEQLNKIYERVKEFGDLGKYISDADLLAIVREVTGKL

SEQ ID NO: 59 - DNA, 1092 bp, Escherichia coli
atgtcgaagaattaccatattgccgtattgccgggggacggtattggtccggaagtgatgacccaggcgctgaaagtgctggatgccgtgcgcaaccgctttgcgatgcgcatcaccaccagccattacgatgtaggcggcgcagccattgataaccacgggcaaccactgccgcctgcgacggttgaaggttgtgagcaagccgatgccgtgctgtttggctcggtaggcggcccgaagtgggaacatttaccaccagaccagcaaccagaacgcggcgcgctgctgcctctgcgtaagcacttcaaattattcagcaacctgcgcccggcaaaactgtatcaggggctggaagcattctgtccgctgcgtgcagacattgccgcaaacggcttcgacatcctgtgtgtgcgcgaactgaccggcggcatctatttcggtcagccaaaaggccgcgaaggtagcggacaatatgaaaaagcctttgataccgaggtgtatcaccgttttgagatcgaacgtatcgcccgcatcgcgtttgaatctgctcgcaagcgtcgccacaaagtgacgtcgatcgataaagccaacgtgctgcaatcctctattttatggcgggagatcgttaacgagatcgccacggaatacccggatgtcgaactggcgcatatgtacatcgacaacgccaccatgcagctgattaaagatccatcacagtttgacgttctgctgtgctccaacctgtttggcgacattctgtctgacgagtgcgcaatgatcactggctcgatggggatgttgccttccgccagcctgaacgagcaaggttttggactgtatgaaccggcgggcggctcggcaccagatatcgcaggcaaaaacatcgccaacccgattgcacaaatcctttcgctggcactgctgctgcgttacagcctggatgccgatgatgcggcttgcgccattgaacgcgccattaaccgcgcattagaagaaggcattcgcaccggggatttagcccgtggcgctgccgccgttagtaccgatgaaatgggcgatatcattgcccgctatgtagcagaaggggtgtaa

SEQ ID NO: 60 - DNA, 1401 bp, Escherichia coli
atggctaagacgttatacgaaaaattgttcgacgctcacgttgtgtacgaagccgaaaacgaaaccccactgttatatatcgaccgccacctggtgcatgaagtgacctcaccgcaggcgttcgatggtctgcgcgcccacggtcgcccggtacgtcagccgggcaaaaccttcgctaccatggatcacaacgtctctacccagaccaaagacattaatgcctgcggtgaaatggcgcgtatccagatgcaggaactgatcaaaaactgcaaagaatttggcgtcgaactgtatgacctgaatcacccgtatcaggggatcgtccacgtaatggggccggaacagggcgtcaccttgccggggatgaccattgtctgcggcgactcgcataccgccacccacggcgcgtttggcgcactggcctttggtatcggcacttccgaagttgaacacgtactggcaacgcaaaccctgaaacagggccgcgcaaaaaccatgaaaattgaagtccagggcaaagccgcgccgggcattaccgcaaaagatatcgtgctggcaattatcggtaaaaccggtagcgcaggcggcaccgggcatgtggtggagttttgcggcgaagcaatccgtgatttaagcatggaaggtcgtatgaccctgtgcaatatggcaatcgaaatgggcgcaaaagccggtctggttgcaccggacgaaaccacctttaactatgtcaaaggccgtctgcatgcgccgaaaggcaaagatttcgacgacgccgttgcctactggaaaaccctgcaaaccgacgaaggcgcaactttcgataccgttgtcactctgcaagcagaagaaatttcaccgcaggtcacctggggcaccaatcccggccaggtgatttccgtgaacgacaatattcccgatccggcttcgtttgccgatccggttgaacgcgcgtcggcagaaaaagcgctggcctatatggggctgaaaccgggtattccgctgaccgaagtggctatcgacaaagtgtttatcggttcctgtaccaactcgcgcattgaagatttacgcgcggcagcggagatcgccaaagggcgaaaagtcgcgccaggcgtgcaggcactggtggttcccggctctggcccggtaaaagcccaggcggaagcggaaggtctggataaaatctttattgaagccggttttgaatggcgcttgcctggctgctcaatgtgtctggcgatgaacaacgaccgtctgaatccgggcgaacgttgtgcctccaccagcaaccgtaactttgaaggccgccaggggcgcggcgggcgcacgcatctggtcagcccggcaatggctgccgctgctgctgtgaccggacatttcgccgacattcgcaacattaaataa

SEQ ID NO: 61 - DNA, 606 bp, Escherichia coli
atggcagagaaatttatcaaacacacaggcctggtggttccgctggatgccgccaatgtcgataccgatgcaatcatcccgaaacagtttttgcagaaagtgacccgtacgggttttggcgcgcatctgtttaacgactggcgttttctggatgaaaaaggccaacagccaaacccggacttcgtgctgaacttcccgcagtatcagggcgcttccattttgctggcacgagaaaacttcggctgtggctcttcgcgtgagcacgcgccctgggcattgaccgactacggttttaaagtggtgattgcgccgagttttgctgacatcttctacggcaatagctttaacaaccagctgctgccggtgaaattaagcgatgcagaagtggacgaactgtttgcgctggtgaaagctaatccggggatccatttcgacgtggatctggaagcgcaagaggtgaaagcgggagagaaaacctatcgctttaccatcgatgccttccgccgccactgcatgatgaacggtctggacagtattgggcttaccttgcagcacgacgacgccattgccgcttatgaagcaaaacaacctgcgtttatgaattaa

SEQ ID NO: 62 - DNA, 3119 bp, Escherichia coli
atgtcgaagaattaccatattgccgtattgccgggggacggtattggtccggaagtgatgacccaggcgctgaaagtgctggatgccgtgcgcaaccgctttgcgatgcgcatcaccaccagccattacgatgtaggcggcgcagccattgataaccacgggcaaccactgccgcctgcgacggttgaaggttgtgagcaagccgatgccgtgctgtttggctcggtaggcggcccgaagtgggaacatttaccaccagaccagcaaccagaacgcggcgcgctgctgcctctgcgtaagcacttcaaattattcagcaacctgcgcccggcaaaactgtatcaggggctggaagcattctgtccgctgcgtgcagacattgccgcaaacggcttcgacatcctgtgtgtgcgcgaactgaccggcggcatctatttcggtcagccaaaaggccgcgaaggtagcggacaatatgaaaaagcctttgataccgaggtgtatcaccgttttgagatcgaacgtatcgcccgcatcgcgtttgaatctgctcgcaagcgtcgccacaaagtgacgtcgatcgataaagccaacgtgctgcaatcctctattttatggcgggagatcgttaacgagatcgccacggaatacccggatgtcgaactggcgcatatgtacatcgacaacgccaccatgcagctgattaaagatccatcacagtttgacgttctgctgtgctccaacctgtttggcgacattctgtctgacgagtgcgcaatgatcactggctcgatggggatgttgccttccgccagcctgaacgagcaaggttttggactgtatgaaccggcgggcggctcggcaccagatatcgcaggcaaaaacatcgccaacccgattgcacaaatcctttcgctggcactgctgctgcgttacagcctggatgccgatgatgcggcttgcgccattgaacgcgccattaaccgcgcattagaagaaggcattcgcaccggggatttagcccgtggcgctgccgccgttagtaccgatgaaatgggcgatatcattgcccgctatgtagcagaaggggtgtaatcatggctaagacgttatacgaaaaattgttcgacgctcacgttgtgtacgaagccgaaaacgaaaccccactgttatatatcgaccgccacctggtgcatgaagtgacctcaccgcaggcgttcgatggtctgcgcgcccacggtcgcccggtacgtcagccgggcaaaaccttcgctaccatggatcacaacgtctctacccagaccaaagacattaatgcctgcggtgaaatggcgcgtatccagatgcaggaactgatcaaaaactgcaaagaatttggcgtcgaactgtatgacctgaatcacccgtatcaggggatcgtccacgtaatggggccggaacagggcgtcaccttgccggggatgaccattgtctgcggcgactcgcataccgccacccacggcgcgtttggcgcactggcctttggtatcggcacttccgaagttgaacacgtactggcaacgcaaaccctgaaacagggccgcgcaaaaaccatgaaaattgaagtccagggcaaagccgcgccgggcattaccgcaaaagatatcgtgctggcaattatcggtaaaaccggtagcgcaggcggcaccgggcatgtggtggagttttgcggcgaagcaatccgtgatttaagcatggaaggtcgtatgaccctgtgcaatatggcaatcgaaatgggcgcaaaagccggtctggttgcaccggacgaaaccacctttaactatgtcaaaggccgtctgcatgcgccgaaaggcaaagatttcgacgacgccgttgcctactggaaaaccctgcaaaccgacgaaggcgcaactttcgataccgttgtcactctgcaagcagaagaaatttcaccgcaggtcacctggggcaccaatcccggccaggtgatttccgtgaacgacaatattcccgatccggcttcgtttgccgatccggttgaacgcgcgtcggcagaaaaagcgctggcctatatggggctgaaaccgggtattccgctgaccgaagtggctatcgacaaagtgtttatcggttcctgtaccaactcgcgcattgaagatttacgcgcggcagcggagatcgccaaagggcgaaaagtcgcgccaggcgtgcaggcactggtggttcccggctctggcccggtaaaagcccaggcggaagcggaaggtctggataaaatctttattgaagccggttttgaatggcgcttgcctggctgctcaatgtgtctggcgatgaacaacgaccgtctgaatccgggcgaacgttgtgcctccaccagcaaccgtaactttgaaggccgccaggggcgcggcgggcgcacgcatctggtcagcccggcaatggctgccgctgctgctgtgaccggacatttcgccgacattcgcaacattaaataaggagcacaccatggcagagaaatttatcaaacacacaggcctggtggttccgctggatgccgccaatgtcgataccgatgcaatcatcccgaaacagtttttgcagaaagtgacccgtacgggttttggcgcgcatctgtttaacgactggcgttttctggatgaaaaaggccaacagccaaacccggacttcgtgctgaacttcccgcagtatcagggcgcttccattttgctggcacgagaaaacttcggctgtggctcttcgcgtgagcacgcgccctgggcattgaccgactacggttttaaagtggtgattgcgccgagttttgctgacatcttctacggcaatagctttaacaaccagctgctgccggtgaaattaagcgatgcagaagtggacgaactgtttgcgctggtgaaagctaatccggggatccatttcgacgtggatctggaagcgcaagaggtgaaagcgggagagaaaacctatcgctttaccatcgatgccttccgccgccactgcatgatgaacggtctggacagtattgggcttaccttgcagcacgacgacgccattgccgcttatgaagcaaaacaacctgcgtttatgaattaagcggccgc

SEQ ID NO: 63 - DNA, 25 bp, Artificial Sequence
  Synthetic primer
ttggtccggaagtgatgacccaggc

SEQ ID NO: 64 - DNA, 43 bp, Artificial Sequence
  Synthetic primer
tatgtgcggccgcttaattcataaacgcaggttgttttgcttc

SEQ ID NO: 65 - protein, 363 aa, Escherichia coli
MSKNYHIAVLPGDGIGPEVMTQALKVLDAVRNRFAMRITTSHYDVGGAAIDNHGQPLPPATVEGCEQADAVLFGSVGGPKWEHLPPDQQPERGALLPLRKHFKLFSNLRPAKLYQGLEAFCPLRADIAANGFDILCVRELTGGIYFGQPKGREGSGQYEKAFDTEVYHRFEIERIARIAFESARKRRHKVTSIDKANVLQSSILWREIVNEIATEYPDVELAHMYIDNATMQLIKDPSQFDVLLCSNLFGDILSDECAMITGSMGMLPSASLNEQGFGLYEPAGGSAPDIAGKNIANPIAQILSLALLLRYSLDADDAACAIERAINRALEEGIRTGDLARGAAAVSTDEMGDIIARYVAEGV

SEQ ID NO: 66 - protein, 466 aa, Escherichia coli
MAKTLYEKLFDAHVVYEAENETPLLYIDRHLVHEVTSPQAFDGLRAHGRPVRQPGKTFATMDHNVSTQTKDINACGEMARIQMQELIKNCKEFGVELYDLNHPYQGIVHVMGPEQGVTLPGMTIVCGDSHTATHGAFGALAFGIGTSEVEHVLATQTLKQGRAKTMKIEVQGKAAPGITAKDIVLAIIGKTGSAGGTGHVVEFCGEAIRDLSMEGRMTLCNMAIEMGAKAGLVAPDETTFNYVKGRLHAPKGKDFDDAVAYWKTLQTDEGATFDTVVTLQAEEISPQVTWGTNPGQVISVNDNIPDPASFADPVERASAEKALAYMGLKPGIPLTEVAIDKVFIGSCTNSRIEDLRAAAEIAKGRKVAPGVQALVVPGSGPVKAQAEAEGLDKIFIEAGFEWRLPGCSMCLAMNNDRLNPGERCASTSNRNFEGRQGRGGRTHLVSPAMAAAAAVTGHFADIRNIK

SEQ ID NO: 67 - protein, 201 aa, Escherichia coli
MAEKFIKHTGLVVPLDAANVDTDAIIPKQFLQKVTRTGFGAHLFNDWRFLDEKGQQPNPDFVLNFPQYQGASILLARENFGCGSSREHAPWALTDYGFKVVIAPSFADIFYGNSFNNQLLPVKLSDAEVDELFALVKANPGIHFDVDLEAQEVKAGEKTYRFTIDAFRRHCMMNGLDSIGLTLQHDDAIAAYEAKQPAFMN

SEQ ID NO: 68 - DNA, 566 bp, Escherichia coli
catgccatggcggacacgttattgattctgggtgatagcctgagcgccgggtatcgaatgtctgccagcgcggcctggcctgccttgttgaatgataagtggcagagtaaaacgtcggtagttaatgccagcatcagcggcgacacctcgcaacaaggactggcgcgccttccggctctgctgaaacagcatcagccgcgttgggtgctggttgaactgggcggcaatgacggtttgcgtggttttcagccacagcaaaccgagcaaacgctgcgccagattttgcaggatgtcaaagccgccaacgctgaaccattgttaatgcaaatacgtctgcctgcaaactatggtcgccgttataatgaagcctttagcgccatttaccccaaactcgccaaagagtttgatgttccgctgctgcccttttttatggaagaggtctacctcaagccacaatggatgcaggatgacggtattcatcccaaccgcgacgcccagccgtttattgccgactggatggcgaagcagttgcagcctttagtaaatcatgactcataaggatccgc

SEQ ID NO: 69 - DNA, 32 bp, Artificial Sequence
  Synthetic primer
catgccatggcggacacgttattgattctggg

SEQ ID NO: 70 - protein, 9 aa, Artificial Sequence
  Synthetic peptide
MADTLLILG

SEQ ID NO: 71 - DNA, 36 bp, Artificial Sequence
  Synthetic primer
gcggatccttatgagtcatgatttactaaaggctgc

SEQ ID NO: 72 - protein, 9 aa, Artificial Sequence
  Synthetic peptide
SDHNVLPQL

SEQ ID NO: 73 - protein, 183 aa, Escherichia coli
MADTLLILGDSLSAGYRMSASAAWPALLNDKWQSKTSVVNASISGDTSQQGLARLPALLKQHQPRWVLVELGGNDGLRGFQPQQTEQTLRQILQDVKAANAEPLLMQIRLPANYGRRYNEAFSAIYPKLAKEFDVPLLPFFMEEVYLKPQWMQDDGIHPNRDAQPFIADWMAKQLQPLVNHDS

SEQ ID NO: 74 - DNA, 33 bp, Artificial Sequence
  Synthetic primer
cattactcgagcgcactcccgttctggataatg

SEQ ID NO: 75 - DNA, 34 bp, Artificial Sequence
  Synthetic primer
gggaagcttatgagtcatgatttactaaaggctg

SEQ ID NO: 76 - protein, 9 aa, Artificial Sequence
  Synthetic peptide
SDHNVLPQL

SEQ ID NO: 77 - DNA, 939 bp, Listeria monocytogenes
atgaacgcaggaattttaggagtaggtaaatacgtacctgaaaaaatagtaactaactttgatttagaaaaaattatggatacatctgatgagtggattcgaactcgaactggtatcgaagaaagaagaattgctcgtgatgacgaatatacgcatgatttagcatatgaagcagcaaaggtagccattaagaatgctggtcttacaccagatgatatcgacttgtttatcgttgcaactgttacacaagaagcgacatttccttcagttgctaatattatccaagaccgtttaggagcgaaaaatgctgccggtatggacgttgaggcagcttgtgctggttttacttttggagtagtgactgcagcacaattcattaaaacaggtgcatataaaaatatcgtagttgtcggtgcggataaattatctaaaatcactaactgggatgatcgcacaacagccgtattattcggtgatggagcaggtgctgtagttatggggccagtttctgatgatcatggtttactttcatttgacttaggttcagatggatcaggtggtaaatacttgaatttagatgaaaataaaaagatttatatgaatggtcgtgaagtgttccgttttgcagttcgccaaatgggagaagcttcactacgagtacttgaacgtgctggtcttgagaaagaagacttggacttactaattcctcaccaagcaaatattcgtatcatggaagcttctcgtgagcgtttgaatttaccagaagaaaaactgatgaaaacagtacacaaatatggtaatacttcatcctcttcaatcgcacttgcgctagttgatgcagtggaagaaggacgtattaaagataatgataacgttctgcttgtaggctttggcggcggactaacatggggcgccctaatcattcgttggggtaagtaa

SEQ ID NO: 78 - DNA, 951 bp, Listeria monocytogenes
ctcgagatgaatgcaggtattctgggtgttggtaaatatgtgccggaaaaaatcgtgaccaacttcgatctggaaaaaattatggataccagcgacgaatggattcgtacccgtaccggtattgaagaacgtcgtattgcacgtgatgatgaatatacccatgatctggcatatgaagcagcaaaagtggcaattaaaaatgcaggtctgacaccggatgatattgacctgtttattgttgcaaccgttacccaagaagcaacctttccgagcgttgcaaatattattcaggatcgtctgggtgcaaaaaatgcagcaggtatggatgttgaagcagcatgtgcaggttttacctttggtgttgttaccgcagcccagtttattaaaaccggtgcctataaaaacatcgttgttgttggtgcagataaactgagcaaaattaccaattgggatgatcgtaccaccgcagttctgtttggtgatggtgccggtgcagttgttatgggtccggttagtgatgatcatggtctgctgtcatttgatctgggtagtgatggtagcggtggtaaatatctgaatctggacgagaataaaaaaatttatatgaatggtcgcgaagtgtttcgttttgcagttcgtcagatgggtgaagcaagcctgcgtgttctggaacgtgcaggtctggaaaaagaggatctggatctgctgattccgcatcaggcaaatattcgtattatggaagcaagccgtgaacgtctgaatctgccggaagaaaaactgatgaaaaccgtgcataaatatggcaataccagcagcagcagcattgcactggcactggttgatgcagttgaagaaggtcgcattaaagataatgataacgttctgctggttggttttggtggtggtctgacctggggtgcactgattattcgttggggtaaataactgcag

SEQ ID NO: 79 - DNA, 20 bp, Artificial Sequence
  Synthetic primer
atgccatagcatttttatcc

SEQ ID NO: 80 - DNA, 21 bp, Artificial Sequence
  Synthetic primer
tctgatttaatctgtatcagg

SEQ ID NO: 81 - DNA, 1476 bp, Escherichia coli
atggctaactacttcaatacactgaatctgcgccagcagctggcacagctgggcaaatgtcgctttatgggccgcgatgaattcgccgatggcgcgagctaccttcagggtaaaaaagtagtcatcgtcggctgtggcgcacagggtctgaaccagggcctgaacatgcgtgattctggtctcgatatctcctacgctctgcgtaaagaagcgattgccgagaagcgcgcgtcctggcgtaaagcgaccgaaaatggttttaaagtgggtacttacgaagaactgatcccacaggcggatctggtgattaacctgacgccggacaagcagcactctgatgtagtgcgcaccgtacagccactgatgaaagacggcgcggcgctgggctactcgcacggtttcaacatcgtcgaagtgggcgagcagatccgtaaagatatcaccgtagtgatggttgcgccgaaatgcccaggcaccgaagtgcgtgaagagtacaaacgtgggttcggcgtaccgacgctgattgccgttcacccggaaaacgatccgaaaggcgaaggcatggcgattgccaaagcctgggcggctgcaaccggtggtcaccgtgcgggtgtgctggaatcgtccttcgttgcggaagtgaaatctgacctgatgggcgagcaaaccatcctgtgcggtatgttgcaggctggctctctgctgtgcttcgacaagctggtggaagaaggtaccgatccagcatacgcagaaaaactgattcagttcggttgggaaaccatcaccgaagcactgaaacagggcggcatcaccctgatgatggaccgtctctctaacccggcgaaactgcgtgcttatgcgctttctgaacagctgaaagagatcatggcacccctgttccagaaacatatggacgacatcatctccggcgaattctcttccggtatgatggcggactgggccaacgatgataagaaactgctgacctggcgtgaagagaccggcaaaaccgcgtttgaaaccgcgccgcagtatgaaggcaaaatcggcgagcaggagtacttcgataaaggcgtactgatgattgcgatggtgaaagcgggcgttgaactggcgttcgaaaccatggtcgattccggcatcattgaagagtctgcatattatgaatcactgcacgagctgccgctgattgccaacaccatcgcccgtaagcgtctgtacgaaatgaacgtggttatctctgataccgctgagtacggtaactatctgttctcttacgcttgtgtgccgttgctgaaaccgtttatggcagagctgcaaccgggcgacctgggtaaagctattccggaaggcgcggtagataacgggcaactgcgtgatgtgaacgaagcgattcgcagccatgcgattgagcaggtaggtaagaaactgcgcggctatatgacagatatgaaacgtattgctgttgcgggttaa

SEQ ID NO: 82 - DNA, 1851 bp, Escherichia coli
atgcctaagtaccgttccgccaccaccactcatggtcgtaatatggcgggtgctcgtgcgctgtggcgcgccaccggaatgaccgacgccgatttcggtaagccgattatcgcggttgtgaactcgttcacccaatttgtaccgggtcacgtccatctgcgcgatctcggtaaactggtcgccgaacaaattgaagcggctggcggcgttgccaaagagttcaacaccattgcggtggatgatgggattgccatgggccacggggggatgctttattcactgccatctcgcgaactgatcgctgattccgttgagtatatggtcaacgcccactgcgccgacgccatggtctgcatctctaactgcgacaaaatcaccccggggatgctgatggcttccctgcgcctgaatattccggtgatctttgtttccggcggcccgatggaggccgggaaaaccaaactttccgatcagatcatcaagctcgatctggttgatgcgatgatccagggcgcagacccgaaagtatctgactcccagagcgatcaggttgaacgttccgcgtgtccgacctgcggttcctgctccgggatgtttaccgctaactcaatgaactgcctgaccgaagcgctgggcctgtcgcagccgggcaacggctcgctgctggcaacccacgccgaccgtaagcagctgttccttaatgctggtaaacgcattgttgaattgaccaaacgttattacgagcaaaacgacgaaagtgcactgccgcgtaatatcgccagtaaggcggcgtttgaaaacgccatgacgctggatatcgcgatgggtggatcgactaacaccgtacttcacctgctggcggcggcgcaggaagcggaaatcgacttcaccatgagtgatatcgataagctttcccgcaaggttccacagctgtgtaaagttgcgccgagcacccagaaataccatatggaagatgttcaccgtgctggtggtgttatcggtattctcggcgaactggatcgcgcggggttactgaaccgtgatgtgaaaaacgtacttggcctgacgttgccgcaaacgctggaacaatacgacgttatgctgacccaggatgacgcggtaaaaaatatgttccgcgcaggtcctgcaggcattcgtaccacacaggcattctcgcaagattgccgttgggatacgctggacgacgatcgcgccaatggctgtatccgctcgctggaacacgcctacagcaaagacggcggcctggcggtgctctacggtaactttgcggaaaacggctgcatcgtgaaaacggcaggcgtcgatgacagcatcctcaaattcaccggcccggcgaaagtgtacgaaagccaggacgatgcggtagaagcgattctcggcggtaaagttgtcgccggagatgtggtagtaattcgctatgaaggcccgaaaggcggtccggggatgcaggaaatgctctacccaaccagcttcctgaaatcaatgggtctcggcaaagcctgtgcgctgatcaccgacggtcgtttctctggtggcacctctggtctttccatcggccacgtctcaccggaagcggcaagcggcggcagcattggcctgattgaagatggtgacctgatcgctatcgacatcccgaaccgtggcattcagttacaggtaagcgatgccgaactggcggcgcgtcgtgaagcgcaggacgctcgaggtgacaaagcctggacgccgaaaaatcgtgaacgtcaggtctcctttgccctgcgtgcttatgccagcctggcaaccagcgccgacaaaggcgcggtgcgcgataaatcgaaactggggggttaa

SEQ ID NO: 83 - DNA, 3682 bp, Escherichia coli
ctcgagcgcactcccgttctggataatgttttttgcgccgacatcataacggttctggcaaatattctgaaatgagctgttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgtttaactttattattaaaaattaaagaggtatatattaatgtatcgattaaataaggaggaataaaccatggctaactactttaacaccctgaacctgcgtcagcaactggctcaactgggcaaatgtcgttttatgggccgtgatgaatttgcggatggtgcttcatatctgcaaggcaaaaaagtggttattgtgggttgcggtgcgcagggcctgaaccaaggtctgaatatgcgtgattcaggtctggacatttcgtatgcactgcgcaaagaagccatcgcagaaaaacgtgcatcgtggcgcaaagctaccgaaaatggctttaaagtgggtacgtatgaagaactgattccgcaggcggatctggtgatcaacctgaccccggataaacagcatagtgacgtcgtgcgtacggttcaaccgctgatgaaagatggcgcggccctgggttattcccacggctttaatattgtcgaagtgggtgaacagattcgcaaagacatcaccgttgtcatggtggcgccgaaatgtccgggcacggaagttcgtgaagaatacaaacgcggcttcggtgtcccgaccctgattgccgtgcatccggaaaacgatccgaaaggcgagggtatggctatcgcaaaagcctgggcagctgcgaccggcggtcaccgtgcaggtgtgctggaaagctctttcgttgccgaagtcaaaagcgatctgatgggcgaacagaccattctgtgcggtatgctgcaagccggctctctgctgtgttttgataaactggttgaagaaggtacggacccggcctatgctgaaaaactgattcagttcggctgggaaaccatcacggaagcgctgaaacaaggcggtattaccctgatgatggatcgtctgagtaatccggcaaaactgcgtgcatacgccctgtccgaacagctgaaagaaatcatggcaccgctgttccaaaaacatatggatgacatcatctccggtgaatttagcagcggcatgatggcagattgggctaacgatgacaaaaaactgctgacctggcgcgaagaaaccggcaaaacggcgtttgaaacggccccgcagtatgaaggcaaaatcggtgaacaagaatacttcgataaaggtgttctgatgatcgcaatggtgaaagctggcgttgaactggcctttgaaaccatggtggatagcggtattatcgaagaaagcgcatattacgaatctctgcatgaactgccgctgattgcgaacaccatcgcccgtaaacgcctgtacgaaatgaacgtggttatctcagatacggcagaatatggcaactacctgttttcgtacgcttgcgtgccgctgctgaaaccgttcatggcggaactgcaaccgggcgacctgggtaaagcgatcccggaaggtgccgttgataacggccagctgcgtgacgtcaatgaagcaattcgcagccacgctatcgaacaagtgggtaaaaaactgcgtggctacatgacggatatgaaacgtattgcggttgcgggctgatcatgccgaaatatcgctctgctacgacgacgcatggccgcaatatggctggtgctcgcgctctgtggcgtgctacgggtatgacggatgctgattttggcaaaccgattatcgcggtggtgaacagctttacccagttcgtcccgggccatgtgcacctgcgtgatctgggtaaactggtggccgaacagatcgaagcggccggcggtgtggcaaaagaatttaataccatcgctgttgatgacggcattgcgatgggtcatggcggtatgctgtattcgctgccgagccgtgaactgattgccgattcggttgaatacatggtgaacgcacactgtgctgacgcgatggtttgtatcagcaattgcgataaaattaccccgggtatgctgatggcctccctgcgcctgaacatcccggttatttttgtctcaggcggtccgatggaagcaggcaaaacgaaactgagcgaccagattatcaaactggacctggtggatgccatgatccaaggtgcagatccgaaagtgagcgactctcagagtgatcaagttgaacgttccgcatgcccgacctgtggctcctgctcaggcatgtttaccgctaactcaatgaattgcctgacggaagcgctgggtctgtctcagccgggtaacggtagtctgctggccacccatgcagatcgtaaacaactgttcctgaatgcgggtaaacgtattgttgaactgacgaaacgctattacgaacagaacgacgaatctgccctgccgcgcaatatcgcaagtaaagcagcttttgaaaacgctatgaccctggatattgcgatgggcggttccaccaatacggtgctgcacctgctggcggcggcacaggaagccgaaattgatttcaccatgtccgacattgataaactgtcacgtaaagtcccgcagctgtgtaaagtggcgccgagcacccaaaaatatcatatggaagatgttcaccgtgccggcggtgtcattggcatcctgggtgaactggaccgtgcaggcctgctgaaccgcgatgtgaaaaatgttctgggtctgaccctgccgcagacgctggaacaatacgatgtcatgctgacccaggatgacgcagtgaaaaacatgttccgtgctggcccggcgggtatccgcaccacgcaggcgttctctcaagactgtcgttgggatacgctggatgacgatcgtgctaatggctgcattcgctcgctggaacatgcgtatagcaaagatggcggtctggccgttctgtacggcaactttgcagaaaatggttgtatcgtgaaaaccgctggcgtggatgattctattctgaaattcacgggtccggcgaaagtttatgaaagtcaggacgatgccgtcgaagcaatcctgggcggtaaagtcgtggcgggcgatgttgtcgtgattcgttatgaaggcccgaaaggcggtccgggtatgcaggaaatgctgtacccgacctcgtttctgaaaagcatgggcctgggtaaagcctgcgcactgatcaccgatggtcgcttctcgggcggtacgtctggcctgagtattggtcacgtgtcaccggaagctgcgtccggcggttcaatcggcctgattgaagacggtgatctgattgcgatcgacattccgaaccgcggcattcagctgcaagtctctgatgccgaactggcagcacgtcgcgaagctcaggacgcgcgtggtgataaagcctggaccccgaaaaatcgtgaacgccaagtgagttttgcactgcgcgcttatgcgagtctggcgacgagtgccgacaaaggtgctgtgcgtgataaatccaaactgggtggctaagatctgcagctggtaccgcggccgcgtttaaacgaattctagaagcttacgcgt

SEQ ID NO: 84 - protein, 491 aa, Escherichia coli
MANYFNTLNLRQQLAQLGKCRFMGRDEFADGASYLQGKKVVIVGCGAQGLNQGLNMRDSGLDISYALRKEAIAEKRASWRKATENGFKVGTYEELIPQADLVINLTPDKQHSDVVRTVQPLMKDGAALGYSHGFNIVEVGEQIRKDITVVMVAPKCPGTEVREEYKRGFGVPTLIAVHPENDPKGEGMAIAKAWAAATGGHRAGVLESSFVAEVKSDLMGEQTILCGMLQAGSLLCFDKLVEEGTDPAYAEKLIQFGWETITEALKQGGITLMMDRLSNPAKLRAYALSEQLKEIMAPLFQKHMDDIISGEFSSGMMADWANDDKKLLTWREETGKTAFETAPQYEGKIGEQEYFDKGVLMIAMVKAGVELAFETMVDSGIIEESAYYESLHELPLIANTIARKRLYEMNVVISDTAEYGNYLFSYACVPLLKPFMAELQPGDLGKAIPEGAVDNGQLRDVNEAIRSHAIEQVGKKLRGYMTDMKRIAVAG

SEQ ID NO: 85 - protein, 616 aa, Escherichia coli
MPKYRSATTTHGRNMAGARALWRATGMTDADFGKPIIAVVNSFTQFVPGHVHLRDLGKLVAEQIEAAGGVAKEFNTIAVDDGIAMGHGGMLYSLPSRELIADSVEYMVNAHCADAMVCISNCDKITPGMLMASLRLNIPVIFVSGGPMEAGKTKLSDQIIKLDLVDAMIQGADPKVSDSQSDQVERSACPTCGSCSGMFTANSMNCLTEALGLSQPGNGSLLATHADRKQLFLNAGKRIVELTKRYYEQNDESALPRNIASKAAFENAMTLDIAMGGSTNTVLHLLAAAQEAEIDFTMSDIDKLSRKVPQLCKVAPSTQKYHMEDVHRAGGVIGILGELDRAGLLNRDVKNVLGLTLPQTLEQYDVMLTQDDAVKNMFRAGPAGIRTTQAFSQDCRWDTLDDDRANGCIRSLEHAYSKDGGLAVLYGNFAENGCIVKTAGVDDSILKFTGPAKVYESQDDAVEAILGGKVVAGDVVVIRYEGPKGGPGMQEMLYPTSFLKSMGLGKACALITDGRFSGGTSGLSIGHVSPEAASGGSIGLIEDGDLIAIDIPNRGIQLQVSDAELAARREAQDARGDKAWTPKNRERQVSFALRAYASLATSADKGAVRDKSKLGG

SEQ ID NO: 86 - protein, 258 aa, Bacillus subtilis
MNFSLEGRNIVVMGVANKRSIAWGIARSLHEAGARLIFTYAGERLEKSVHELAGTLDRNDSIILPCDVTNDAEIETCFASIKEQVGVIHGIAHCIAFANKEELVGEYLNTNRDGFLLAHNISSYSLTAVVKAARPMMTEGGSIVTLTYLGGELVMPNYNVMGVAKASLDASVKYLAADLGKENIRVNSISAGPIRTLSAKGISDFNSILKDIEERAPLRRTTTPEEVGDTAAFLFSDMSRGITGENLHVDSGFHITAR

SEQ ID NO: 87 - DNA, 37 bp, Artificial Sequence
  Synthetic primer
gagaccatggatgaatttttcacttgaaggccgtaac

SEQ ID NO: 88 - DNA, 46 bp, Artificial Sequence
  Synthetic primer
gagacgctgcagttagcgggcagtgatatggaaaccagaatcaacg

SEQ ID NO: 89 - DNA, 789 bp, Listeria monocytogenes
ccatggatgaatttttcacttgaaggccgtaacattgttgtgatgggggtagccaacaaacgcagcatcgcctggggcattgcgcgttctttacatgaagcgggtgcacgtttgattttcacatacgctggtgaacgcctggagaaatccgttcacgagcttgccggaacattagaccgcaacgattccatcatcctcccttgcgatgttacaaacgacgcagaaatcgaaacttgcttcgcaagcattaaggagcaggtcggtgtaatccacggtatcgcgcattgtatcgcgtttgccaacaaagaagagcttgtcggcgagtacttaaacacaaatcgtgacggcttccttttggctcataacatcagctcatattctctgactgctgttgtcaaagcggcacgtccgatgatgactgaaggcggaagcattgtcactttgacgtaccttggcggagagcttgtgatgccaaactacaacgtcatgggtgtagcaaaagcttctcttgatgcaagtgtgaaatatttagctgctgacttaggaaaagaaaatatccgcgtcaacagcatttctgccggcccgatcagaacattatctgctaaaggcatcagcgatttcaactctatcttaaaagacatcgaagagcgtgcaccgcttcgccgcacgacaacacctgaagaagtgggcgatacagctgcgttcttgttcagcgatatgtcccgcgggattacaggtgaaaatcttcacgttgattctggtttccatatcactgcccgctaactgcag

SEQ ID NO: 90 - DNA, 789 bp, Listeria monocytogenes
ctcgagatgaatttttcacttgaaggccgtaacattgttgtgatgggggtagccaacaaacgcagcatcgcctggggcattgcgcgttctttacatgaagcgggtgcacgtttgattttcacatacgctggtgaacgcctggagaaatccgttcacgagcttgccggaacattagaccgcaacgattccatcatcctcccttgcgatgttacaaacgacgcagaaatcgaaacttgcttcgcaagcattaaggagcaggtcggtgtaatccacggtatcgcgcattgtatcgcgtttgccaacaaagaagagcttgtcggcgagtacttaaacacaaatcgtgacggcttccttttggctcataacatcagctcatattctctgactgctgttgtcaaagcggcacgtccgatgatgactgaaggcggaagcattgtcactttgacgtaccttggcggagagcttgtgatgccaaactacaacgtcatgggtgtagcaaaagcttctcttgatgcaagtgtgaaatatttagctgctgacttaggaaaagaaaatatccgcgtcaacagcatttctgccggcccgatcagaacattatctgctaaaggcatcagcgatttcaactctatcttaaaagacatcgaagagcgtgcaccgcttcgccgcacgacaacacctgaagaagtgggcgatacagctgcgttcttgttcagcgatatgtcccgcgggattacaggtgaaaatcttcacgttgattctggtttccatatcactgcccgctaactgcag

SEQ ID NO: 91 - DNA, 39 bp, Artificial Sequence
  Synthetic primer
gagacgctcgagatgaatttttcacttgaaggccgtaac

SEQ ID NO: 92 - protein, 258 aa, Escherichia coli; identical to SEQ ID NO: 86